(19) United States
(12) Patent Application Publication
(10) Pub. No.: US 2026/0130999 A1
(43) Pub. Date: May 14, 2026

(54) METHODS FOR TREATING CANCERS ASSOCIATED WITH ETBR ACTIVATION

(71) Applicant: ENB Therapeutics, Inc., New York, NY (US)

(72) Inventor: Sumayah Jamal, New York, NY (US)

(21) Appl. No.: 19/125,242

(22) PCT Filed: Oct. 27, 2023
(86) PCT No.: PCT/US2023/078095

§ 371 Date: Apr. 28, 2025

Related U.S. Application Data
(60) Provisional application No. 63/381,542, filed on Oct. 28, 2022.

(51) Int. Cl. A61K 40/42; A61K 38/07; A61K 39/00; A61K 39/395; A61K 40/11; A61K 40/15; A61K 40/31; A61P 35/00

(52) U.S. Cl. CPC A61K 40/4205; A61K 38/07; A61K 39/3955; A61K 40/11; A61K 40/15; A61K 40/31; A61P 35/00; A61K 2039/505

(57) ABSTRACT

Disclosed herein are methods for treating cancers with a combination therapy comprising a CAR-expressing immune cell (e.g., a CAR-T cell) and an ETBR antagonist. Also disclosed herein are methods for treating cancers with a combination therapy comprising a CAR-expressing immune cell (e.g., a CAR-T cell), an immune checkpoint inhibitor (e.g., an anti-PD1 antibody), and an ETBR antagonist.

### CROSS-REFERENCE TO RELATED APPLICATIONS

This application is the U.S. National Stage entry of International Application No. PCT/US2023/078095, filed on Oct. 27, 2023, which claims the benefit of and priority to U.S. Provisional Application No. 63/381,542, filed on Oct. 28, 2022, the disclosure of each of which is hereby incorporated by reference in their entireties for all purposes.

### SUMMARY

Disclosed herein, in certain embodiments are methods of treating a target-expressing cancer in a subject in need thereof, the method comprising administering to the subject a combination comprising: (a) a chimeric antigen receptor (CAR)-expressing immune cell, wherein the CAR specifically binds the target; and (B) an effective amount of an ETBR antagonist.

Disclosed herein, in various embodiments of a method of treating a target-expressing cancer in a subject in need thereof with a chimeric antigen receptor (CAR)-expressing immune cell, wherein the CAR specifically binds the target, is an improvement comprising co-administering to the subject an effective amount of an ETBR antagonist.

Disclosed herein, in certain embodiments are methods of treating a target-expressing cancer in a subject in need thereof, the method comprising administering to the subject a combination comprising: (a) a chimeric antigen receptor (CAR)-expressing immune cell, wherein the CAR specifically binds the target; (b) an immune checkpoint inhibitor (ICPI); and (c) an effective amount of an ETBR antagonist.

Disclosed herein, in various embodiments of a method of treating a target-expressing cancer in a subject in need thereof with a chimeric antigen receptor (CAR)-expressing immune cell, wherein the CAR specifically binds the target, and an immune checkpoint inhibitor (ICPI), provided is an improvement comprising co-administering to the subject an effective amount of an ETBR antagonist.

In some embodiments, the target is a tumor-associated antigen. In some embodiments, the tumor-associated antigen is HER2.

Disclosed herein, in certain embodiments are methods of treating a HER2-expressing cancer in a subject in need thereof, the method comprising administering to the subject a combination comprising: (a) a chimeric antigen receptor (CAR)-expressing immune cell, wherein the CAR specifically binds HER2, and (b) an effective amount of an ETBR antagonist.

Disclosed herein, in various embodiments of a method of treating a HER2-expressing cancer in a subject in need thereof with a chimeric antigen receptor (CAR)-expressing immune cell, wherein the CAR specifically binds HER2, is an improvement comprising co-administering to the subject an effective amount of an ETBR antagonist.

In some embodiments, the ICPI is an anti-PD1 antibody, an anti-PD-L1 antibody, an anti-CTLA4 antibody, or a variant or functional fragment thereof. In some embodiments, the ICPI is an anti-PD1 antibody.

In some embodiments, the ETBR antagonist is a deuterated ETBR antagonist.

In some embodiments, the deuterated ETBR antagonist is a compound of formula (1):

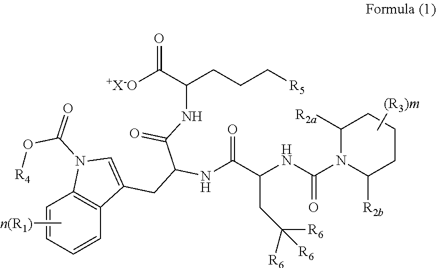

or a stereoisomer thereof, wherein:
n is an integer from 0-4;
m is an integer from 0-3;
X is a positively charged counterion; R₁ and R₃ are independently —H, -D, —CH₃, —CH₂D, —CHD₂, or —CD₃; R₂ₐ, R_{2b}, R₄, R₅, and R₆ are independently —CH₃, —CH₂D, —CHD₂, or —CD₃; and at least one of R₁, R₂ₐ, R_{2b}, or R₃ comprises deuterium.

In some embodiments, the compound is a compound of Formula (1a):

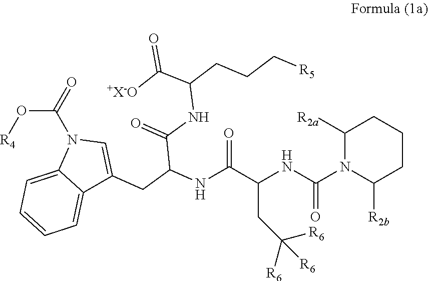

or a stereoisomer thereof.

In some embodiments, the compound is a compound of Formula (2):

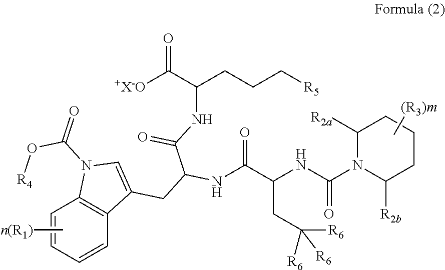

or a stereoisomer thereof.

In some embodiments, the compound is a compound of Formula (2a):

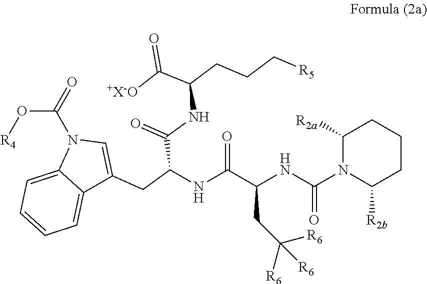

or a stereoisomer thereof.

In some embodiments, the compound is a compound of Formula (3):

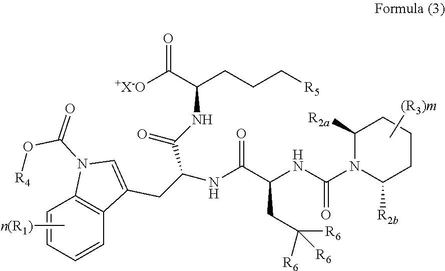

or a stereoisomer thereof.

In some embodiments, the compound is a compound of Formula (3a):

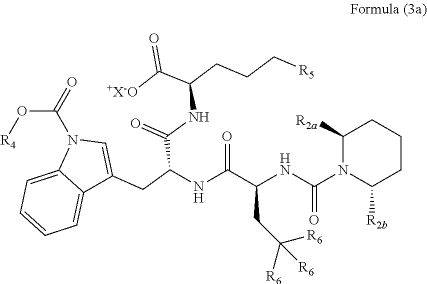

or a stereoisomer thereof.

In some embodiments, the compound is a compound of Formula (4):

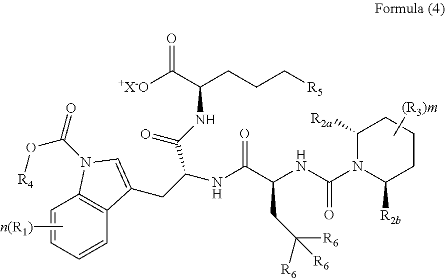

or a stereoisomer thereof.

In some embodiments, the compound is a compound of Formula (4a):

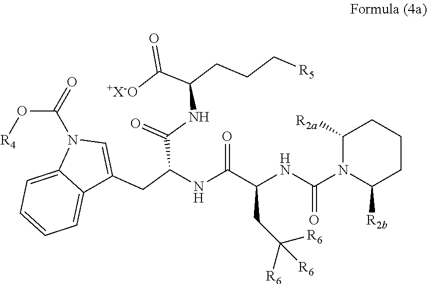

or a stereoisomer thereof.

In some embodiments, the compound is a compound of Formula (5):

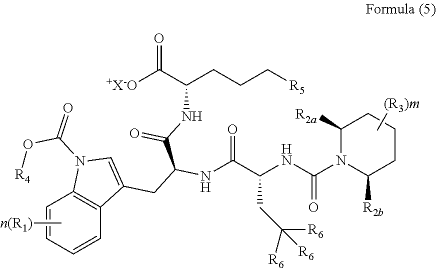

or a stereoisomer thereof.

In some embodiments, the compound is a compound of Formula (5a):

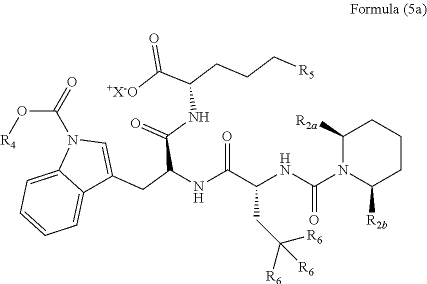

or a stereoisomer thereof.

In some embodiments, the compound is a compound of Formula (6):

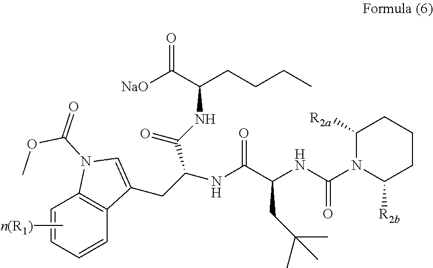

or a stereoisomer thereof.

In some embodiments, the compound is a compound of Formula (6a):

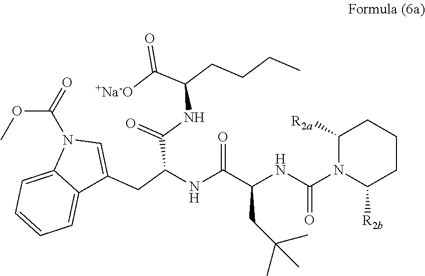

or a stereoisomer thereof.

In some embodiments, n is 1, R₁ is -D, and R₂ₐ and R_{2b} are each —CH₃.

In some embodiments, R₂ₐ is —CH₃, and R_{2b} is —CH₂D.

In some embodiments, R₂ₐ and R_{2b} are each —CH₂D.

In some embodiments, n is 1, R₁ is -D; and R₂ₐ and R_{2b} are each —CH₂D.

In some embodiments, the deuterated ETBR antagonist is:

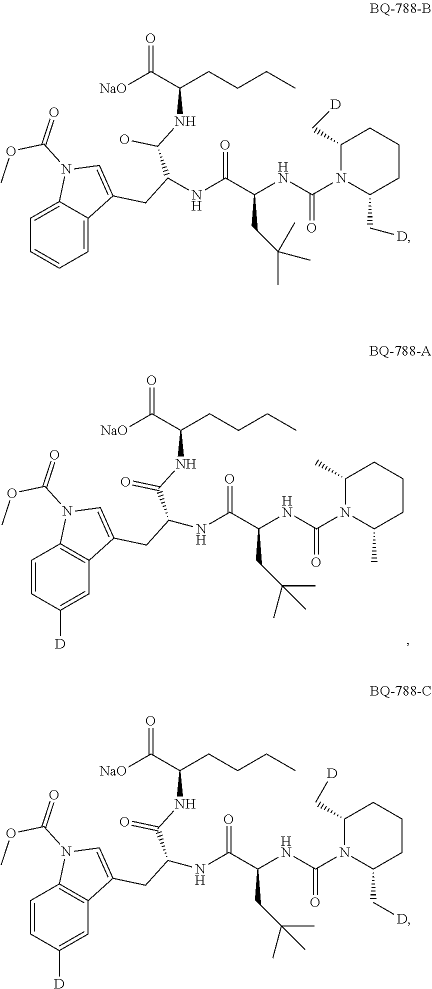

or a stereoisomer thereof.

In some embodiments, the ETBR antagonist is:

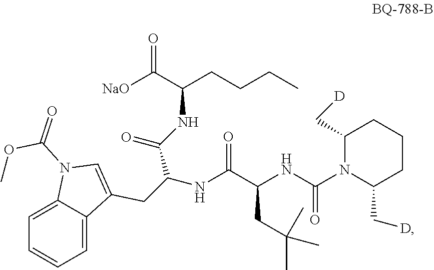

or a stereoisomer thereof.

In some embodiments, the ETBR antagonist is in a pharmaceutically acceptable excipient that comprises dimethyl sulfoxide (DMSO).

In some embodiments, the CAR-expressing immune cell is a CAR-T cell or a CAR-NK cell. In some embodiments, the CAR-expressing immune cell is a CAR-T cell. In some embodiments, the cancer is selected from the group consisting of: breast cancer, gastric cancer, esophageal cancer, ovarian cancer, endometrial cancer, bladder cancer, lung cancer, cervical cancer, colon cancer, and head and neck cancer.

In some embodiments, the cancer is an ETBR-related cancer.

In some embodiments, the ETBR-related cancer is selected from the group consisting of: breast cancer, gastric cancer, esophageal cancer, ovarian cancer, endometrial cancer, bladder cancer, lung cancer, cervical cancer, colon cancer, and head and neck cancer.

In some embodiments, the cancer is a HER2-expressing cancer.

In some embodiments, the HER2-expressing cancer is a HER2+ cancer.

In some embodiments, the HER2-expressing cancer is a HER2-low cancer.

In some embodiments, the HER2-expressing cancer is selected from the group consisting of: breast cancer, gastric cancer, esophageal cancer, ovarian cancer, endometrial cancer, bladder cancer, lung cancer, cervical cancer, colon cancer, and head and neck cancer.

### BRIEF DESCRIPTION OF THE DRAWINGS

The accompanying drawings, which are incorporated into and form a part of the specification, illustrate several embodiments of the present invention and, together with the description, serve to explain the principles of the invention. The drawings are only for the purpose of illustrating an embodiment of the invention and are not to be construed as limiting the invention. Further objects, features and advantages of the invention will become apparent from the following detailed description taken in conjunction with the accompanying figures showing illustrative embodiments of the invention, in which:

FIG. 1 shows endothelin B receptor (ETBR) cell signal pathway. ETBR is a seven transmembrane G-protein coupled receptor (GPCR). Endothelin-1 (ET-1) is the ligand for the ETBR. Binding of ET-1 to the receptor results in the activation of a number of downstream kinases, including PTK, RAF, MEK, MAPK/ERK.

FIG. 2 depicts tumor volumes over time in 4T1 engrafted mice treated with indicated combination therapies.

FIGS. 3A and 3B depict tumor growth from day 11 to day 17 post-4T1 tumor engraftment in mice treated with indicated combination therapies. FIG. 3A depicts tumor volumes over time. FIG. 3B depicts tumor volumes at day 17 post-tumor engraftment. The differences between group 8 and groups 1 and 2, respectively, have a P<0.01. The differences between group 8 and groups 3 and 4, respectively, have a P<0.05.

FIG. 4 depicts tumor volumes at day 29 post-4T1 tumor engraftment in mice treated with indicated combination therapies. The difference between groups 1 and 2 has a P<0.05. The difference between groups 1 and 4 has a P<0.01. The difference between groups 4 and 6 has a P<0.01. The differences between group 1 and groups 5, 6, 7 and 8, respectively, have a P<0.001. The differences between group 2 and groups 5, 6, 7 and 8, respectively, have a P<0.001. The differences between group 3 and groups 5, 6, 7 and 8, respectively, have a P<0.001. The differences between group 4 and groups 5, 7 and 8, respectively, have a P<0.001.

FIGS. 5A and 5B depict tumor growth at experimental endpoint (day 30 post-4T1 tumor engraftment) in mice treated with indicated combination therapies. FIG. 5A shows images of harvested tumors. FIG. 5B depicts tumor weights at day 30 post-tumor engraftment. The difference between groups 1 and 4 has a P<0.05. The differences between group 4 and groups 5, 6 and 7, respectively, have a P<0.05. The difference between groups 4 and 8 has a P<0.01. The differences between group 3 and groups 6 and 7 have a P<0.01. The differences between group 1 and groups 5, 6, 7 and 8, respectively, have a P<0.001. The differences between group 2 and groups 5, 6, 7 and 8, respectively, have a P<0.001. The differences between group 3 and groups 5 and 8, respectively, have a P<0.001.

### DETAILED DESCRIPTION

Disclosed herein are combination therapies and methods of treating cancer, in a single dosage form or separate dosage forms administered concurrently or separately, comprising an ETBR antagonist as described herein and a CAR-expressing immune cell (e.g., a CAR-T cell). In some embodiments, the therapeutic combination further comprises an immune checkpoint inhibitor (ICPI), e.g., an anti-PD1 antibody or anti-PD-L1 antibody. In some embodiments, the ETBR antagonist is a specifically deuterated ETBR antagonist as described herein. In some embodiments, the specifically deuterated ETBR antagonist is deuterated BQ-788, BQ-788-A, BQ-788-B, or BQ-788-C or a combination thereof.

In some embodiments, the combination therapies as described herein demonstrate a synergistic effect in that the pharmaceutical compositions achieve at least one of: a greater therapeutic effect (i.e., more efficacious) than the additive therapeutic effect obtained by administration of the constituent ingredients alone, a greater therapeutic effect than achieved by administration of a higher dose of the constituent ingredients alone, a similar or greater therapeutic effect but with a decrease in adverse events or side effects relative to that observed by administration of the constituent ingredients alone (i.e., improved therapeutic window), or increased duration of effects, or a similar or greater therapeutic effect at a smaller dose of one or both of the constituent ingredients or a combination thereof.

### Definitions

Unless otherwise defined, all technical and scientific terms used herein have the same meaning as commonly understood by one of ordinary skill in the art to which this invention belongs. The terminology used in the description is for describing particular embodiments only and is not intended to be limiting of the invention.

Where a range of values is provided, it is understood that each intervening value, to the tenth of the unit of the lower limit unless the context clearly dictates otherwise (such as in the case of a group containing a number of carbon atoms in which case each carbon atom number falling within the range is provided), between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the present disclosure.

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, in certain methods described herein that include more than one step or act, the order of the steps or acts of the method is not necessarily limited to the order in which the steps or acts of the method are recited unless the context indicates otherwise.

The term “combination therapy” refers to both concurrent administration (administration of two or more therapeutic agents at the same time) and time varied administration (administration of one or more therapeutic agents at a time different from that of the administration of an additional therapeutic agent or agents). In some embodiments, the therapeutic agents are present in the patient to some extent, for example at effective amounts, at the same time. In some embodiments, one or more of the compounds described herein, are administered in combination with an additional bioactive agent, especially including an anticancer agent. In some embodiments, the combination therapy of compounds results in synergistic activity, including anticancer activity.

The term “compound,” as used herein, unless otherwise indicated, refers to any specific chemical compound disclosed herein and includes tautomers, regioisomers, geometric isomers, and where applicable, stereoisomers, including optical isomers (enantiomers) and other steroisomers (diastereomers) thereof, as well as pharmaceutically acceptable salts and derivatives (including prodrug forms) thereof where applicable, in context. Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds. The term also refers, in context to prodrug forms of compounds which have been modified to facilitate the administration and delivery of compounds to a site of activity. It is noted that in describing the present compounds, numerous substituents and variables associated with same, among others, are described. It is understood by those of ordinary skill that molecules which are described herein are stable compounds as generally described hereunder. When the bond is shown, both a double bond and single bond are represented within the context of the compound shown.

The terms “treat,” “treating,” and “treatment,” etc., as used herein, refer to any action providing a benefit to a patient for which the present compounds may be administered, including the treatment of any disease state or condition which is modulated through the protein to which the present compounds bind. Disease states or conditions, including cancer, which may be treated using compounds according to the present disclosure, are set forth hereinabove.

The term “pharmaceutically acceptable salt” is used throughout the specification to describe, where applicable, a salt form of one or more of the compounds described herein which are presented to increase the solubility of the compound in the gastric juices of the patient's gastrointestinal tract in order to promote dissolution and the bioavailability of the compounds. Pharmaceutically acceptable salts include those derived from pharmaceutically acceptable inorganic or organic bases and acids, where applicable. Suitable salts include those derived from alkali metals such as potassium and sodium, alkaline earth metals such as calcium, magnesium and ammonium salts. In some embodiments, sodium and potassium salts are suitable neutralization salts of the phosphates.

The term “pharmaceutically acceptable derivative” is used throughout the specification to describe any pharmaceutically acceptable prodrug form (such as an ester, amide or other prodrug group), which, upon administration to a patient, provides directly or indirectly the present compound or an active metabolite of the present compound.

The term “effective” is used to describe an amount of a compound, composition or component which, when used within the context of its intended use, effects an intended result. The term “effective” subsumes all other effective amount or effective concentration terms, which are otherwise described or used in the present application.

The term “therapeutically effective amount” refers to that amount which is sufficient to effect treatment, as defined herein, when administered to a mammal in need of such treatment.

The term “patient” or “subject” is used throughout the specification to describe an animal, for example a human, or a domesticated animal, to whom treatment, including prophylactic treatment, with the compositions according to the present disclosure is provided. For treatment of those infections, conditions or disease states which are specific for a specific animal such as a human patient, the term patient refers to that specific animal, including a domesticated animal such as a dog or cat or a farm animal such as a horse, cow, sheep, etc. In general, in the present disclosure, the term patient refers to a human patient unless otherwise stated or implied from the context of the use of the term. Activation of the ETBR by endothelins such as ET-1 and ET-3, results in a variety of molecular events that promote melanoma invasion and metastasis. Without being bound by any particular theory, it is hypothesized that while the majority of melanomas express ETBR, a subset of these also expresses the ETBR activator ET-1 and/or ET-3. It is this subset that is therefore most likely dependent upon ETBR activation for viability, invasive potential and metastatic potential. Thus, this subset of patients is most likely to respond to ETBR blockade. Furthermore, this subset of patients is least likely to respond to immune based therapy.

### Deuterated ETBR Antagonists

In some embodiments, the methods and pharmaceutical compositions described herein use or comprise an deuterated ETBR antagonist, e.g., a deuterated form of BQ-788 as described herein. The specific deuterated compounds referenced herein are beneficial because, as compared to nondeuterated versions of the same compounds, there is an improvement in one or more pharmaceutical properties (e.g., efficacy, solubility). The specifically deuterated ETBR antagonists, including deuterated forms of BQ-788 (e.g., BQ-788A, BQ-788-B and BQ-788-C) referenced herein have been described in WO2019140324, which is incorporated herein by reference in its entirety. The specifically deuterated ETBR antagonist BQ-788-B is also known as ENB-003.

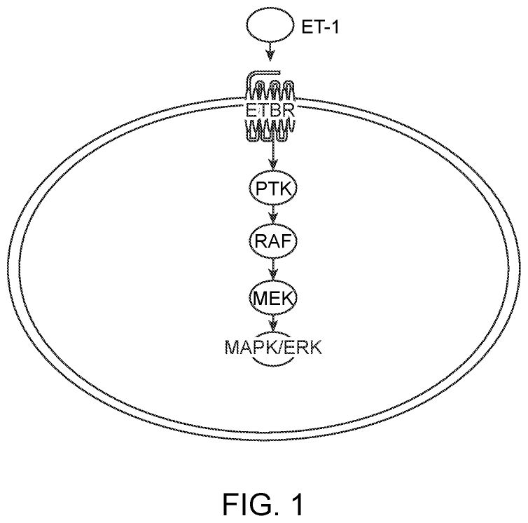

The Endothelin B receptor (ETBR) pathway (FIG. 1) plays a significant role in the metastatic spread of melanoma, and therefore, is a target for therapeutic intervention. The Endothelin B receptor is a 7 transmembrane G-protein coupled receptor (GPCR). It is expressed at very low levels in normal melanocytes, but is upregulated during melanoma development and progression. RAF and MEK kinases, current melanoma drug targets, are activated by the deuterated ETBR. In some embodiments, the physiologic role of the ETBR is to clear excess levels of endothelin-1 (ET-1), from the circulation. Without being bound by any particular theory, it is hypothesized that administering an ETBR antagonist (e.g., a specifically deuterated ETBR antagonist) prevents ET-1 clearance and elevates serum ET-1 levels. Elevated serum levels of ET-1 are associated with a variety of adverse effects due to its activation of the Endothelin A receptor (ETAR) including, hypertension, pulmonary hypertension and renal vasoconstriction.

In some embodiments, the description provides a composition comprising an ETBR antagonist (e.g., a specifically deuterated ETBR antagonist, e.g., a deuterated form of BQ-788 as described herein), and a pharmaceutically acceptable carrier. In some embodiments, the description provides a composition, e.g., a pharmaceutical composition, comprising an effective amount of an ETBR antagonist (e.g., a specifically deuterated ETBR antagonist, e.g., a deuterated form of BQ-788 as described herein), and a pharmaceutically acceptable carrier. In some embodiments, the pharmaceutical composition as described herein can be in unit dosage form configured for administration one or more times, for example, one or more times per day, per week, or per month.

In some embodiments, the specifically deuterated ETBR antagonist is a compound of the Formula (1) below:

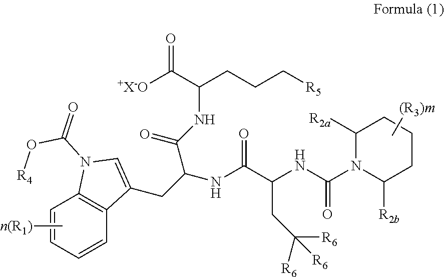

wherein
n is an integer from 0-5;
m is an integer from 0-3;
X is a positively charged counterion;
R₁ and R₃ are independently —H, -D, —CH₃, —CH₂D, —CHD₂, or —CD₃;
R₂ₐ, R_{2b}, R₄, R₅, and R₆ are independently —CH₃, —CH₂D, —CHD₂, or —CD₃; and
at least one of R₁, R₂, or R₃ comprises deuterium.

In some embodiments, the specifically deuterated ETBR antagonist of formula (1) comprises 1-8 deuterium atoms. In specific embodiments, the specifically deuterated ETBR antagonist of formula (1) comprises 1, 2, or 3 deuterium atoms.

In some embodiments, the compound is of formula (1a) below:

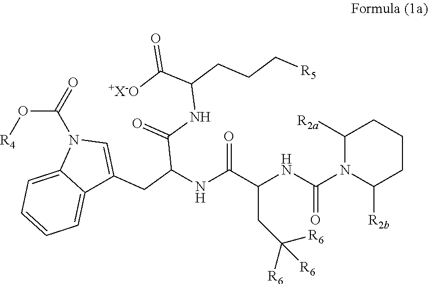

wherein X, R₂ₐ, R_{2b}, R₄, R₅ and R₆ are as described herein.

In some embodiments, the specifically deuterated ETBR antagonist is a compound of the Formula (2) below:

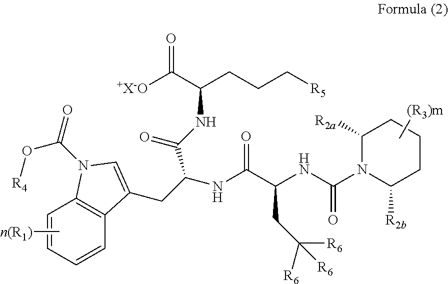

wherein X, m, n, R₁, R₃, R₂ₐ, R_{2b}, R₄, R₅ and R₆ are as described herein.

In some embodiments, the compound is of formula (2a) below:

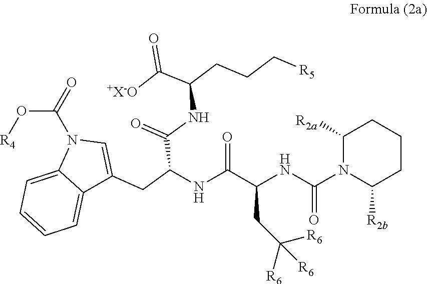

wherein X, R₂ₐ, R_{2b}, R₄, R₅ and R₆ are as described herein.

In some embodiments, the specifically deuterated ETBR antagonist is a compound of the Formula (3) below:

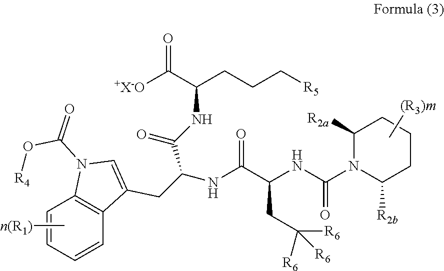

wherein X, m, n, R₁, R₃, R₂ₐ, R_{2b}, R₄, R₅ and R₆ are as described herein.

In some embodiments, the compound is of formula (3a) below:

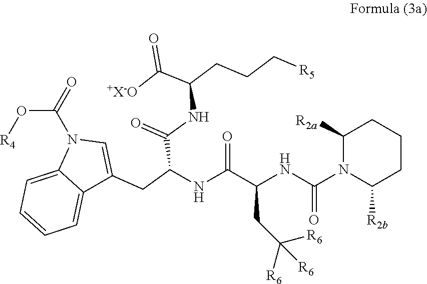

wherein X, R₂ₐ, R_{2b}, R₄, R₅ and R₆ are as described herein.

In some embodiments, the specifically deuterated ETBR antagonist is a compound of the Formula (4) below:

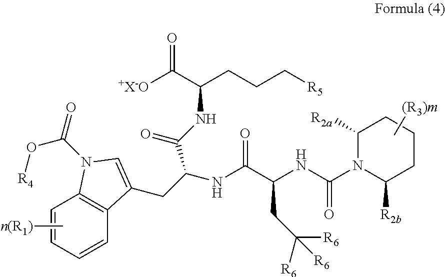

wherein X, m, n, R₁, R₃, R₂ₐ, R_{2b}, R₄, R₅ and R₆ are as described herein.

In some embodiments, the compound is of formula (4a) below:

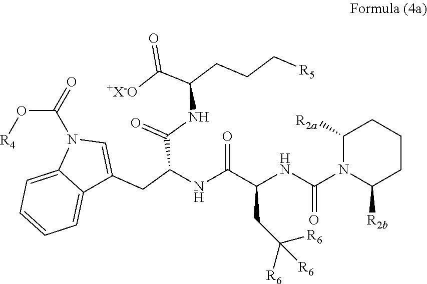

wherein X, R₂ₐ, R_{2b}, R₄, R₅ and R₆ are as described herein.

In some embodiments, the specifically deuterated ETBR antagonist is a compound of the Formula (5) below:

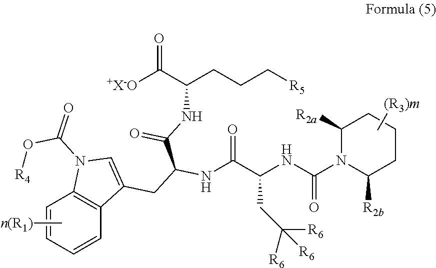

wherein X, m, n, R₁, R₃, R₂ₐ, R_{2b}, R₄, R₅ and R₆ are as described herein.

In some embodiments, the compound is of formula (5a) below:

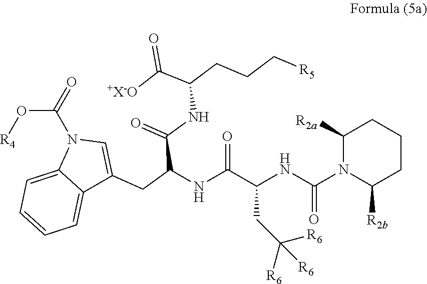

wherein X, R₂ₐ, R_{2b}, R₄, R₅ and R₆ are as described herein.

In some embodiments, the specifically deuterated ETBR antagonist is a compound of the formula (6) below:

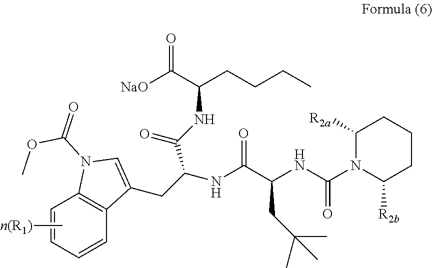

wherein n, R₁, R₂ₐ and R_{2b}, are as described herein.

In some embodiments, the compound is of formula (6a) below:

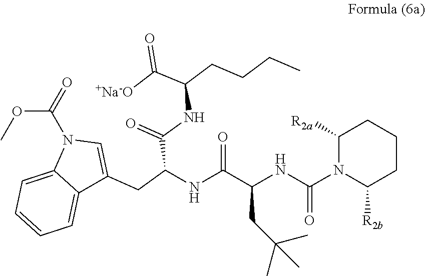

wherein R₂ₐ and R_{2b}, are as described herein.

In some embodiments of the specifically deuterated ETBR antagonist of formulae (1) to (6), n is 0 or 1. In some embodiments, n is 0. In some embodiments, n is 1.

In some embodiments of the specifically deuterated ETBR antagonist of formulae (1) to (5), m is 0 or 1. In some embodiments, m is 0. In some embodiments, m is 1.

In some embodiments of the specifically deuterated ETBR antagonist of formulae (1)-(6), n is 1 and R₁ is -D.

In some embodiments of the specifically deuterated ETBR antagonist of formulae (1) to (5a), X is selected from Na⁺ and K⁺. In some embodiments, X is Na⁺.

In some embodiments of the specifically deuterated ETBR antagonist of formulae (1)-(6a), R₂ₐ and R_{2b} are —CH₂D.

In some embodiments of the specifically deuterated ETBR antagonist of formulae (1)-(6a), R₂ₐ and R_{2b} are —CH₃.

In some embodiments of the specifically deuterated ETBR antagonist of formulae (1)-(6a), one of R₂ₐ or R_{2b} is CH₂D and the other is —CH₃.

In some embodiments, the specifically deuterated ETBR antagonist is BQ-788-A, BQ-788-B, BQ-788-C, or a combination thereof, including analogs, derivatives, polymorphs, prodrugs, and salts thereof, including fluorinated analogues. For example, the specifically deuterated ETBR antagonist can be a fluorinated analog of BQ-788-A, BQ-788-B, or BQ-788-C.

In some embodiments, the specifically deuterated ETBR antagonist is BQ-788-A depicted below:

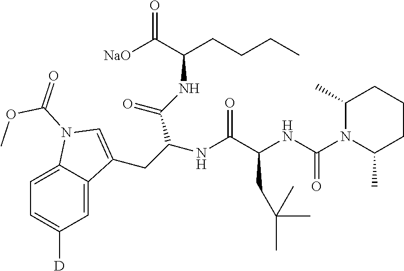

or a stereoisomer thereof.

In some embodiments, the specifically deuterated ETBR antagonist is BQ-788-A.

In some embodiments, the specifically deuterated ETBR antagonist is BQ-788-B depicted below:

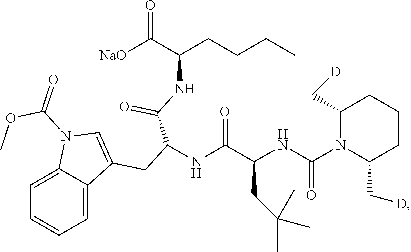

or a stereoisomer thereof.

In some embodiments, the specifically deuterated ETBR antagonist is BQ-788-B (also known as ENB-003).

In some embodiments, the specifically deuterated ETBR antagonist is BQ-788-C depicted below:

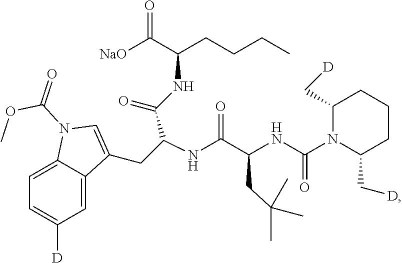

or a stereoisomer thereof.

In some embodiments, the specifically deuterated ETBR antagonist is BQ-788-C.

### Pharmaceutical Compositions

In various embodiments, provided herein are pharmaceutical compositions comprising an ETBR antagonist (e.g., a specifically deuterated ETBR antagonist, e.g., a deuterated form of BQ-788 as described herein), and a pharmaceutically acceptable carrier.

In some embodiments, the compositions herein are formulated in a unit dosage form, including any desired carrier or excipient, and configured for administration via any desired route, e.g., oral, intravenous, subcutaneous, intramuscular, intraperitoneal, parenteral, intranasal, intracranial.

In some embodiments, the pharmaceutical compositions are delivered intravenously, intramuscularly, subcutaneously, orally, intranasally, sublingually, transdermally, topically, intraperitoneally, parenterally, intranasally, or intracranially.

In some embodiments, the ETBR-antagonist, e.g., deuterated ETBR-antagonist or deuterated BQ-788, is administered in the form of a liposomal formulation as described herein.

In some embodiments, the pharmaceutical composition comprises about 1% to about 95% of the active ingredient, single-dose forms of administration comprising about 20% to about 90% of the active ingredient and administration forms which are not single-dose comprising about 5% to about 20% of the active ingredient. Unit dose forms are, for example, coated tablets, tablets, ampoules, vials, suppositories or capsules. Other forms of administration are, for example, ointments, creams, pastes, foams, tinctures, lipsticks, drops, sprays, dispersions and the like. Examples are capsules containing from about 0.05 g to about 5.0 g of the active ingredient.

In some embodiments, the active ingredient is included in the pharmaceutically acceptable carrier or diluent in an amount sufficient to deliver to a patient a therapeutically effective amount for the desired indication, without causing serious toxic effects in the patient treated. An exemplary dose of the active compound for all of the herein-mentioned conditions is in the range from about 10 ng/kg to 300 mg/kg, for example 0.001 to 100 mg/kg/day, more generally 0.01 to about 0.5 mg per kilogram body weight of the recipient/patient per day.

A typical topical dosage will range from 0.01-5% wt/wt in a suitable carrier. The compound is conveniently administered in any suitable unit dosage form, including but not limited to one containing 0.01 mg to 50 mg, 0.01 mg to 40 mg, 0.01 mg to 30 mg, 0.01 mg to 20 mg, 0.01 mg to 15 mg, 0.01 mg to 10 mg, 0.1 mg to 50 mg, 0.1 mg to 40 mg, 0.1 mg to 30 mg, 0.1 mg to 20 mg, 0.1 mg to 15 mg, 0.1 mg to 10 mg, 1 mg to 50 mg, 1 mg to 40 mg, 1 mg to 30 mg, 1 mg to 20 mg, 1 mg to 15 mg, 1 mg to 10 mg, 5 mg to 50 mg, 5 mg to 40 mg, 5 mg to 30 mg, 5 mg to 20 mg, 5 mg to 15 mg, 5 mg to 10 mg, 7.5 mg to 50 mg, 7.5 mg to 40 mg, 7.5 mg to 30 mg, 7.5 mg to 20 mg, 7.5 mg to 15 mg, 7.5 mg to 10 mg, 10 mg to 50 mg, 10 mg to 40 mg, 10 mg to 30 mg or 10 mg to 20 mg of active ingredient per unit dosage form. In some embodiments, the compositions comprise a dosage of the ETBR antagonist (e.g., the specifically deuterated ETBR antagonist) of about 0.1 mg to about 50 mg (e.g., about 1 mg to about 25 mg), and/or a concentration of the ETBR antagonist (e.g., the specifically deuterated ETBR antagonist) of about 0.001 mg/mL to about 10 mg/mL (e.g., about 0.01 mg/mL to about 1 mg/mL).

In some embodiments, the compositions as described herein are formulated in a conventional manner using one or more pharmaceutically acceptable carriers and may also be administered in controlled-release formulations. Pharmaceutically acceptable carriers that may be used in these pharmaceutical compositions include, but are not limited to, dimethyl sulfoxide (DMSO), soybean oil as a carrier, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

In some embodiments, the compositions include soybean oil, dimethyl sulfoxide (DMSO), hydrogel, or a combination thereof. Any of the embodiments described herein can be a single-component oil phase formulation, as described above, wherein each active ingredient can be at any of the dosages or concentrations described herein. The single-component oil phase can be a fixed oil, such as soybean oil. For example, the formulation comprises about 0.1 mg to about 5.0 mg of each active ingredient in 1 mL of the single-component oil (i.e., about 0.5 mg/mL, about 1 mg/mL, or about 1.5 mg/mL of each active ingredient in the single-component oil). The single-component oil phase formulation can be prepared by adding each active ingredient (e.g., about 1 mg to about 50 mg of each of the active ingredient(s)) to about 10 mL of the single-component oil solution.

In some embodiments, pharmaceutical compositions herein comprise a DMSO, e.g., in a DMSO solution that is about 5% to about 100% DMSO (e.g., about 10% to about 100%, about 20% to about 100%, about 30% to about 100%, about 40% to about 100%, about 50% to about 100%, about 60% to about 100%, about 70% to about 100%, about 80% to about 100%, about 90% to about 100%, about 30% to about 95%, about 45% to about 95%, about 75% to about 95%, about 30% to about 90%, about 45% to about 90%, about 75% to about 90%, about 30% to about 85%, about 45% to about 85%, or about 75% to about 85%). For example, the pharmaceutical compositions comprises about 0.01 mg to about 5.0 mg of each active ingredient in 1 mL of DMSO (i.e., about 0.05 mg/mL, about 0.1 mg/mL, about 0.15 mg/mL, about 0.2 mg/mL, about 0.3 mg/mL, about 0.5 mg/mL, about 1 mg/mL, or about 1.5 mg/mL of each active ingredient in DMSO). The DMSO pharmaceutical compositions can be prepared by adding each active ingredient (e.g., about 0.1 mg to about 50 mg of each of the active ingredient(s)) to about 10 mL of the DMSO solution. For example, the DMSO is a DMSO solution comprising about 5% to about 100% DMSO, about 25% to about 100% DMSO, about 50% to about 100% DMSO, about 75% to about 100% DMSO, about 5% to about 75% DMSO, about 25% to about 75% DMSO, about 50% to about 75% DMSO, about 5% to about 50% DMSO, about 25% to about 50% DMSO, or about 5% to about 25% DMSO.

In some embodiments, the description provides a controlled release subcutaneous or intramuscular dosage formulation comprising a uniform dispersion of an ETBR antagonist (e.g., a specifically deuterated ETBR antagonist, e.g., BQ-788, a deuterated or fluorinated analog thereof, or combinations thereof).

In some embodiments, the pharmaceutical composition as described herein is formulated into a controlled release delivery system comprising a biocompatible polymer. In some embodiments, the active compounds are prepared with carriers that will protect the compound against rapid elimination from the body, such as a controlled release formulation, including implants, hydrogels, thermo-sensitive hydrogels, and microencapsulated delivery systems. Biodegradable, biocompatible polymers can be used, such as ethylene vinyl acetate, acrylates, polycarboxylic acids, polyanhydrides, polyglycolic acid, collagen, polyorthoesters, and polylactic acid. In some embodiments, the biocompatible polymer is a poly(lactide), poly(glycolide), poly(lactide-co-glycolide), poly(lactic acid), poly(glycolic acid), poly(lactic acid-co-glycolic acid), polycaprolactone, polycarbonate, polyesteramide, polyanhydride, poly(amino acid), polyorthoester, polycyanoacrylate, poly(p-dioxanone), poly(alkylene oxalate), biodegradable polyurethane, blend, or a copolymer thereof.

In some embodiments, the pharmaceutically acceptable carrier comprises or is a liposome. For example, the pharmaceutical composition or formulation may comprise a liposome having an interior volume comprising an ETBR antagonist (e.g., a specifically deuterated ETBR antagonist). In some embodiments, the liposome is configured to effectuate the controlled release of the ETBR antagonist (e.g., the specifically deuterated ETBR antagonist), e.g., rapid release, extended release, or a combination thereof.

In some embodiments, the liposome is configured to effectuate the controlled release of the pharmaceutical compositions. In some embodiments, the liposome is configured to effectuate rapid release of the pharmaceutical compositions. In other embodiments, the liposome is configured or formulated to effectuate extended release the pharmaceutical compositions. In some embodiments, the liposome is configured to result in both the rapid and extended release of pharmaceutical compositions.

In some embodiments, liposomal suspensions are pharmaceutically acceptable carriers. For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound is then introduced into the container. The container is then swirled by hand to free lipid material from the sides of the container and to disperse lipid aggregates, thereby forming the liposomal suspension. In some embodiments, the liposome comprises a neutral lipid, a basic (having a net positive charge) lipid, an acidic (having a net negative charge) lipid, cholesterol, or a combination thereof. In some embodiments, the liposome further comprises a polymeric component. In some embodiments, the interior volume of the liposome is at least partially aqueous, and comprises an ETBR antagonist (e.g., a specifically deuterated ETBR antagonist).

In some embodiments, the description provides the pharmaceutical composition as described herein in a liposomal delivery system, e.g., a phosphatidylethanolamine (PE) such as dipalmitoyl PE (DPPE), and partially unsaturated phosphatidylcholine (PC), such as egg PC (EPC) or SPC, fully unsaturated PC such as HSPC, PG, phosphatidylserine (PS), phosphatidylinositol (PI) or a combination thereof. In some embodiments, the phospholipid is a partially unsaturated PG, dipalmitoylphosphatidylglycerol (DPPG), cholesterol, DSPE-PEG2000, polysorbate-80 or combination thereof. In some embodiments, the liposomal delivery system is a controlled release system, e.g., at least one of rapid release, extended release, rapid and extended release, delayed release, sustained release, slow release, and combinations thereof.

In some embodiments, the pharmaceutical compositions herein comprise pharmaceutically acceptable salts, in particular, acid or base addition salts of compounds as described herein. The acids which are used to prepare the pharmaceutically acceptable acid addition salts of the aforementioned base compounds useful according to this aspect are those which form non-toxic acid addition salts, i.e., salts containing pharmacologically acceptable anions, such as the hydrochloride, hydrobromide, hydroiodide, nitrate, sulfate, bisulfate, bitartrate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [i.e., 1,1′-methylene-bis-(2-hydroxy-3 naphthoate)]salts, among numerous others. Pharmaceutically acceptable base addition salts may also be used to produce pharmaceutically acceptable salt forms of the compounds or derivatives according to the present disclosure. The chemical bases that may be used as reagents to prepare pharmaceutically acceptable base salts of the present compounds that are acidic in nature are those that form nontoxic base salts with such compounds. Such non-toxic base salts include, but are not limited to those derived from such pharmacologically acceptable cations such as alkali metal cations (e.g., potassium and sodium) and alkaline earth metal cations (e.g., calcium, zinc and magnesium), ammonium or water-soluble amine addition salts such as N-methylglucamine-(meglumine), and the lower alkanolammonium and other base salts of pharmaceutically acceptable organic amines, among others.

In some embodiments, oral compositions include an inert diluent or an edible carrier. They may be enclosed in gelatin capsules or compressed into tablets. For the purpose of oral therapeutic administration, the active compound or its prodrug derivative can be incorporated with excipients and used in the form of tablets, troches, or capsules. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a dispersing agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or enteric agents.

In some embodiments, the active compound or pharmaceutically acceptable salt thereof is administered as a component of an elixir, suspension, syrup, wafer, chewing gum or the like. A syrup may contain, in addition to the active compounds, sucrose as a sweetening agent and certain preservatives, dyes and colorings and flavors.

In some embodiments, solutions or suspensions used for parenteral, intradermal, subcutaneous, intravenous, intramuscular, or topical application include the following components: a sterile diluent such as water for injection, saline solution, fixed oils (e.g., soybean oil), polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic. In some embodiments, carriers for intravenous administration are physiological saline or phosphate buffered saline (PBS).

In some embodiments, each of the active ingredient of a pharmaceutical composition of the present disclosure can be present in any of the dosage formulation (e.g., initial burst, sustained release dosage, etc.) in an amount of about 50 μg to about 100000 μg, about 50 μg to about 90000 μg, about 50 μg to about 80000 μg, about 50 μg to about 70000 μg, about 50 μg to about 60000 μg, about 50 μg to about 50000 μg, about 50 μg to about 40000 μg, about 50 μg to about 30000 μg, about 50 μg to about 25000 μg, about 50 μg to about 20000 μg, about 50 μg to about 15000 μg, about 50 μg to about 10000 μg, about 50 μg to about 9000 μg, about 50 μg to about 8000 μg, about 50 μg to about 7000 μg, about 50 μg to about 6000 μg, about 50 μg to about 5000 μg, about 50 μg to about 4000 μg, about 50 μg to about 3000 μg, about 50 μg to about 2500 μg, about 50 μg to about 2000 μg, about 50 μg to about 1500 μg, about 50 μg to about 1000 μg, about 50 μg to about 750 μg, about 50 μg to about 500 μg, about 50 μg to about 400 μg, about 50 μg to about 300 μg, about 50 μg to about 200 μg, about 50 μg to about 100 μg, about 100 μg to about 100000 μg, about 100 μg to about 90000 μg, about 100 μg to about 80000 μg, about 100 μg to about 70000 μg, about 100 μg to about 60000 μg, about 100 μg to about 50000 μg, about 100 μg to about 40000 μg, about 100 μg to about 30000 μg, about 100 μg to about 25000 μg, about 100 μg to about 20000 μg, about 100 μg to about 15000 μg, about 100 μg to about 10000 μg, about 100 μg to about 9000 μg, about 100 μg to about 8000 μg, about 100 μg to about 7000 μg, about 100 μg to about 6000 μg, about 100 μg to about 5000 μg, about 100 μg to about 4000 μg, about 100 μg to about 3000 μg, about 100 μg to about 2500 μg, about 100 μg to about 2000 μg, about 100 μg to about 1500 μg, about 100 μg to about 1000 μg, about 100 μg to about 750 μg, about 100 μg to about 500 μg, about 100 μg to about 400 μg, about 100 μg to about 300 μg, about 100 μg to about 200 μg, about 200 μg to about 100000 μg, about 200 μg to about 90000 μg, about 200 μg to about 80000 μg, about 200 μg to about 70000 μg, about 200 μg to about 60000 μg, about 200 μg to about 50000 μg, about 200 μg to about 40000 μg, about 200 μg to about 30000 μg, about 200 μg to about 25000 μg, about 200 μg to about 20000 μg, about 200 μg to about 15000 μg, about 200 μg to about 10000 μg, about 200 μg to about 9000 μg, about 200 μg to about 8000 μg, about 200 μg to about 7000 μg, about 200 μg to about 6000 μg, about 200 μg to about 5000 μg, about 200 μg to about 4000 μg, about 200 μg to about 3000 μg, about 200 μg to about 2500 μg, about 200 μg to about 2000 μg, about 200 μg to about 1500 μg, about 200 μg to about 1000 μg, about 200 μg to about 750 μg, about 200 μg to about 500 μg, about 200 μg to about 400 μg, about 200 μg to about 300 μg, about 300 μg to about 100000 μg, about 300 μg to about 90000 μg, about 300 μg to about 80000 μg, about 300 μg to about 70000 μg, about 300 μg to about 60000 μg, about 300 μg to about 50000 μg, about 300 μg to about 40000 μg, about 300 μg to about 30000 μg, about 300 μg to about 25000 μg, about 300 μg to about 20000 μg, about 300 μg to about 15000 μg, about 300 μg to about 10000 μg, about 300 μg to about 9000 μg, about 300 μg to about 8000 μg, about 300 μg to about 7000 μg, about 300 μg to about 6000 μg, about 300 μg to about 5000 μg, about 300 μg to about 4000 μg, about 300 μg to about 3000 μg, about 300 μg to about 2500 μg, about 300 μg to about 2000 μg, about 300 μg to about 1500 μg, about 300 μg to about 1000 μg, about 300 μg to about 750 μg, about 300 μg to about 500 μg, about 300 μg to about 400 μg, about 400 μg to about 100000 μg, about 400 μg to about 90000 μg, about 400 μg to about 80000 μg, about 400 μg to about 70000 μg, about 400 μg to about 60000 μg, about 400 μg to about 50000 μg, about 400 μg to about 40000 μg, about 400 μg to about 30000 μg, about 400 μg to about 25000 μg, about 400 μg to about 20000 μg, about 400 μg to about 15000 μg, about 400 μg to about 10000 μg, about 400 μg to about 9000 μg, about 400 μg to about 8000 μg, about 400 μg to about 7000 μg, about 400 μg to about 6000 μg, about 400 μg to about 5000 μg, about 400 μg to about 4000 μg, about 400 μg to about 3000 μg, about 400 μg to about 2500 μg, about 400 μg to about 2000 μg, about 400 μg to about 1500 μg, about 400 μg to about 1000 μg, about 400 μg to about 750 μg, about 400 μg to about 500 μg, about 500 μg to about 100000 μg, about 500 μg to about 90000 μg, about 500 μg to about 80000 μg, about 500 μg to about 70000 μg, about 500 μg to about 60000 μg, about 500 μg to about 50000 μg, about 500 μg to about 40000 μg, about 500 μg to about 30000 μg, about 500 μg to about 25000 μg, about 500 μg to about 20000 μg, about 500 μg to about 15000 μg, about 500 μg to about 10000 μg, about 500 μg to about 9000 μg, about 500 μg to about 8000 μg, about 500 μg to about 7000 μg, about 500 μg to about 6000 μg, about 500 μg to about 5000 μg, about 500 μg to about 4000 μg, about 500 μg to about 3000 μg, about 500 μg to about 2500 μg, about 500 μg to about 2000 μg, about 500 μg to about 1500 μg, about 500 μg to about 1000 μg, about 500 μg to about 750 μg, about 750 μg to about 100000 μg, about 750 μg to about 90000 μg, about 750 μg to about 80000 μg, about 750 μg to about 70000 μg, about 750 μg to about 60000 μg, about 750 μg to about 50000 μg, about 750 μg to about 40000 μg, about 750 μg to about 30000 μg, about 750 μg to about 25000 μg, about 750 μg to about 20000 μg, about 750 μg to about 15000 μg, about 750 μg to about 10000 μg, about 750 μg to about 9000 μg, about 750 μg to about 8000 μg, about 750 μg to about 7000 μg, about 750 μg to about 6000 μg, about 750 μg to about 5000 μg, about 750 μg to about 4000 μg, about 750 μg to about 3000 μg, about 750 μg to about 2500 μg, about 750 μg to about 2000 μg, about 750 μg to about 1500 μg, about 750 μg to about 1000 μg, about 1000 μg to about 100000 μg, about 1000 μg to about 90000 μg, about 1000 μg to about 80000 μg, about 1000 μg to about 70000 μg, about 1000 μg to about 60000 μg, about 1000 μg to about 50000 μg, about 1000 μg to about 40000 μg, about 1000 μg to about 30000 μg, about 1000 μg to about 25000 μg, about 1000 μg to about 20000 μg, about 1000 μg to about 15000 μg, about 1000 μg to about 10000 μg, about 1000 μg to about 9000 μg, about 1000 μg to about 8000 μg, about 1000 μg to about 7000 μg, about 1000 μg to about 6000 μg, about 1000 μg to about 5000 μg, about 1000 μg to about 4000 μg, about 1000 μg to about 3000 μg, about 1000 μg to about 2500 μg, about 1000 μg to about 2000 μg, about 1000 μg to about 1500 μg, about 1500 μg to about 100000 μg, about 1500 μg to about 90000 μg, about 1500 μg to about 80000 μg, about 1500 μg to about 70000 μg, about 1500 μg to about 60000 μg, about 1500 μg to about 50000 μg, about 1500 μg to about 40000 μg, about 1500 μg to about 30000 μg, about 1500 μg to about 25000 μg, about 1500 μg to about 20000 μg, about 1500 μg to about 15000 μg, about 1500 μg to about 10000 μg, about 1500 μg to about 9000 μg, about 1500 μg to about 8000 μg, about 1500 μg to about 7000 μg, about 1500 μg to about 6000 μg, about 1500 μg to about 5000 μg, about 1500 μg to about 4000 μg, about 1500 μg to about 3000 μg, about 1500 μg to about 2500 μg, about 1500 μg to about 2000 μg, about 2000 μg to about 100000 μg, about 2000 μg to about 90000 μg, about 2000 μg to about 80000 μg, about 2000 μg to about 70000 μg, about 2000 μg to about 60000 μg, about 2000 μg to about 50000 μg, about 2000 μg to about 40000 μg, about 2000 μg to about 30000 μg, about 2000 μg to about 25000 μg, about 2000 μg to about 20000 μg, about 2000 μg to about 15000 μg, about 2000 μg to about 10000 μg, about 2000 μg to about 9000 μg, about 2000 μg to about 8000 μg, about 2000 μg to about 7000 μg, about 2000 μg to about 6000 μg, about 2000 μg to about 5000 μg, about 2000 μg to about 4000 μg, about 2000 μg to about 3000 μg, about 2000 μg to about 2500 μg, about 2500 μg to about 100000 μg, about 2500 μg to about 90000 μg, about 2500 μg to about 80000 μg, about 2500 μg to about 70000 μg, about 2500 μg to about 60000 μg, about 2500 μg to about 50000 μg, about 2500 μg to about 40000 μg, about 2500 μg to about 30000 μg, about 2500 μg to about 25000 μg, about 2500 μg to about 20000 μg, about 2500 μg to about 15000 μg, about 2500 μg to about 10000 μg, about 2500 μg to about 9000 μg, about 2500 μg to about 8000 μg, about 2500 μg to about 7000 μg, about 2500 μg to about 6000 μg, about 2500 μg to about 5000 μg, about 2500 μg to about 4000 μg, about 2500 μg to about 3000 μg, about 3000 μg to about 100000 μg, about 3000 μg to about 90000 μg, about 3000 μg to about 80000 μg, about 3000 μg to about 70000 μg, about 3000 μg to about 60000 μg, about 3000 μg to about 50000 μg, about 3000 μg to about 40000 μg, about 3000 μg to about 30000 μg, about 3000 μg to about 25000 μg, about 3000 μg to about 20000 μg, about 3000 μg to about 15000 μg, about 3000 μg to about 10000 μg, about 3000 μg to about 9000 μg, about 3000 μg to about 8000 μg, about 3000 μg to about 7000 μg, about 3000 μg to about 6000 μg, about 3000 μg to about 5000 μg, about 3000 μg to about 4000 μg, about 4000 μg to about 100000 μg, about 4000 μg to about 90000 μg, about 4000 μg to about 80000 μg, about 4000 μg to about 70000 μg, about 4000 μg to about 60000 μg, about 4000 μg to about 50000 μg, about 4000 μg to about 40000 μg, about 4000 μg to about 30000 μg, about 4000 μg to about 25000 μg, about 4000 μg to about 20000 μg, about 4000 μg to about 15000 μg, about 4000 μg to about 10000 μg, about 4000 μg to about 9000 μg, about 4000 μg to about 8000 μg, about 4000 μg to about 7000 μg, about 4000 μg to about 6000 μg, about 4000 μg to about 5000 μg, about 5000 μg to about 100000 μg, about 5000 μg to about 90000 μg, about 5000 μg to about 80000 μg, about 5000 μg to about 70000 μg, about 5000 μg to about 60000 μg, about 5000 μg to about 50000 μg, about 5000 μg to about 40000 μg, about 5000 μg to about 30000 μg, about 5000 μg to about 25000 μg, about 5000 μg to about 20000 μg, about 5000 μg to about 15000 μg, about 5000 μg to about 10000 μg, about 5000 μg to about 9000 μg, about 5000 μg to about 8000 μg, about 5000 μg to about 7000 μg, about 5000 μg to about 6000 μg, about 6000 μg to about 100000 μg, about 6000 μg to about 90000 μg, about 6000 μg to about 80000 μg, about 6000 μg to about 70000 μg, about 6000 μg to about 60000 μg, about 6000 μg to about 50000 μg, about 6000 μg to about 40000 μg, about 6000 μg to about 30000 μg, about 6000 μg to about 25000 μg, about 6000 μg to about 20000 μg, about 6000 μg to about 15000 μg, about 6000 μg to about 10000 μg, about 6000 μg to about 9000 μg, about 6000 μg to about 8000 μg, about 6000 μg to about 7000 μg, about 7000 μg to about 100000 μg, about 7000 μg to about 90000 μg, about 7000 μg to about 80000 μg, about 7000 μg to about 70000 μg, about 7000 μg to about 60000 μg, about 7000 μg to about 50000 μg, about 7000 μg to about 40000 μg, about 7000 μg to about 30000 μg, about 7000 μg to about 25000 μg, about 7000 μg to about 20000 μg, about 7000 μg to about 15000 μg, about 7000 μg to about 10000 μg, about 7000 μg to about 9000 μg, about 7000 μg to about 8000 μg, about 8000 μg to about 100000 μg, about 8000 μg to about 90000 μg, about 8000 μg to about 80000 μg, about 8000 μg to about 70000 μg, about 8000 μg to about 60000 μg, about 8000 μg to about 50000 μg, about 8000 μg to about 40000 μg, about 8000 μg to about 30000 μg, about 8000 μg to about 25000 μg, about 8000 μg to about 20000 μg, about 8000 μg to about 15000 μg, about 8000 μg to about 10000 μg, about 8000 μg to about 9000 μg, about 9000 μg to about 100000 μg, about 9000 μg to about 90000 μg, about 9000 μg to about 80000 μg, about 9000 μg to about 70000 μg, about 9000 μg to about 60000 μg, about 9000 μg to about 50000 μg, about 9000 μg to about 40000 μg, about 9000 μg to about 30000 μg, about 9000 μg to about 25000 μg, about 9000 μg to about 20000 μg, about 9000 μg to about 15000 μg, about 9000 μg to about 10000 μg, about 10000 μg to about 100000 μg, about 10000 μg to about 90000 μg, about 10000 μg to about 80000 μg, about 10000 μg to about 70000 μg, about 10000 μg to about 60000 μg, about 10000 μg to about 50000 μg, about 10000 μg to about 40000 μg, about 10000 μg to about 30000 μg, about 10000 μg to about 25000 μg, about 10000 μg to about 20000 μg, about 10000 μg to about 15000 μg, about 15000 μg to about 100000 μg, about 15000 μg to about 90000 μg, about 15000 μg to about 80000 μg, about 15000 μg to about 70000 μg, about 15000 μg to about 60000 μg, about 15000 μg to about 50000 μg, about 15000 μg to about 40000 μg, about 15000 μg to about 30000 μg, about 15000 μg to about 25000 μg, about 15000 μg to about 20000 μg, about 20000 μg to about 100000 μg, about 20000 μg to about 90000 μg, about 20000 μg to about 80000 μg, about 20000 μg to about 70000 μg, about 20000 μg to about 60000 μg, about 20000 μg to about 50000 μg, about 20000 μg to about 40000 μg, about 20000 μg to about 30000 μg, about 20000 μg to about 25000 μg, about 30000 μg to about 100000 μg, about 30000 μg to about 90000 μg, about 30000 μg to about 80000 μg, about 30000 μg to about 70000 μg, about 30000 μg to about 60000 μg, about 30000 μg to about 50000 μg, about 30000 μg to about 40000 μg, about 40000 μg to about 100000 μg, about 40000 μg to about 90000 μg, about 40000 μg to about 80000 μg, about 40000 μg to about 70000 μg, about 40000 μg to about 60000 μg, about 40000 μg to about 50000 μg, about 50000 μg to about 100000 μg, about 50000 μg to about 90000 μg, about 50000 μg to about 80000 μg, about 50000 μg to about 70000 μg, about 50000 μg to about 60000 μg, about 60000 μg to about 100000 μg, about 60000 μg to about 90000 μg, about 60000 μg to about 80000 μg, about 60000 μg to about 70000 μg, about 70000 μg to about 100000 μg, about 70000 μg to about 90000 μg, about 70000 μg to about 80000 μg, about 80000 μg to about 100000 μg, about 80000 μg to about 90000 μg or about 90000 μg to about 100000 μg.

In some embodiments, each of the active ingredient of a pharmaceutical composition of the present disclosure can be present in any of the dosage formulation (e.g., initial burst, sustained release dosage, etc.) in an amount of about 50 μg to about 500 μg, about 500 μg to about 2000 μg, about 2000 μg to about 10000 μg, about 10000 μg to about 20000 μg, about 20000 μg to about 50000 μg or about 50000 μg to about 100000 μg.

In some embodiments, each active ingredient of a pharmaceutical composition of the present disclosure is present in about 0.1 mg/mL to about 50 mg/mL, about 0.1 mg/mL to about 25 mg/mL, about 0.1 mg/mL to about 10 mg/mL, about 1 mg/mL to about 50 mg/mL, about 1 mg/mL to about 25 mg/mL, about 1 mg/mL to about 10 mg/mL, about 0.1 mg/mL to about 5.0 mg/mL (e.g., about 0.1 mg/mL to about 4.5 mg/mL, about 0.1 mg/mL to about 4.0 mg/mL, about 0.1 mg/mL to about 3.5 mg/mL, about 0.1 mg/mL to about 3.0 mg/mL, about 0.1 mg/mL to about 2.5 mg/mL, about 0.1 mg/mL to about 2.0 mg/mL, about 0.1 mg/mL to about 1.5 mg/mL, about 0.1 mg/mL to about 1.0 mg/mL, about 0.1 mg/mL to about 0.5 mg/mL, about 0.5 mg/mL to about 4.5 mg/mL, about 0.5 mg/mL to about 4.0 mg/mL, about 0.5 mg/mL to about 3.5 mg/mL, about 0.5 mg/mL to about 3.0 mg/mL, about 0.5 mg/mL to about 2.5 mg/mL, about 0.5 mg/mL to about 2.0 mg/mL, about 0.5 mg/mL to about 1.5 mg/mL, about 0.5 mg/mL to about 1.0 mg/mL, about 1.0 mg/mL to about 4.5 mg/mL, about 1.0 mg/mL to about 4.0 mg/mL, about 1.0 mg/mL to about 3.5 mg/mL, about 1.0 mg/mL to about 3.0 mg/mL, about 1.0 mg/mL to about 2.5 mg/mL, about 1.0 mg/mL to about 2.0 mg/mL, about 1.0 mg/mL to about 1.5 mg/mL, about 1.5 mg/mL to about 4.5 mg/mL, about 1.5 mg/mL to about 4.0 mg/mL, about 1.5 mg/mL to about 3.5 mg/mL, about 1.5 mg/mL to about 3.0 mg/mL, about 1.5 mg/mL to about 2.5 mg/mL, about 1.5 mg/mL to about 2.0 mg/mL, about 2.0 mg/mL to about 4.5 mg/mL, about 2.0 mg/mL to about 4.0 mg/mL, about 2.0 mg/mL to about 3.5 mg/mL, about 2.0 mg/mL to about 3.0 mg/mL, about 2.0 mg/mL to about 2.5 mg/mL, about 2.5 mg/mL to about 4.5 mg/mL, about 2.5 mg/mL to about 4.0 mg/mL, about 2.5 mg/mL to about 3.5 mg/mL, about 2.5 mg/mL to about 3.0 mg/mL, about 3.0 mg/mL to about 4.5 mg/mL, about 3.0 mg/mL to about 4.0 mg/mL, about 3.0 mg/mL to about 3.5 mg/mL, about 3.5 mg/mL to about 4.5 mg/mL, about 3.5 mg/mL to about 4.0 mg/mL, or about 3.5 mg/mL to about 4.5 mg/mL, relative to the pharmaceutical composition).

In some embodiments, each active ingredient of a pharmaceutical composition of the present disclosure is present in about 0.1 μg/mL to about 50 μg/mL, about 0.1 μg/mL to about 25 μg/mL, about 0.1 μg/mL to about 10 μg/mL, about 1 μg/mL to about 50 μg/mL, about 1 μg/mL to about 25 μg/mL, about 1 μg/mL to about 10 μg/mL, about 0.1 μg/mL to about 5.0 μg/mL, e.g., about 1 μg/mL to about 5 μg/mL, about 0.1 μg/mL to about 4.0 μg/mL, about 0.1 μg/mL to about 3.5 μg/mL, about 0.1 μg/mL to about 3.0 μg/mL, about 0.1 μg/mL to about 2.5 μg/mL, about 0.1 μg/mL to about 2.0 μg/mL, about 0.1 μg/mL to about 1.5 μg/mL, about 0.1 μg/mL to about 1.0 μg/mL, about 0.1 μg/mL to about 0.5 μg/mL, about 0.5 μg/mL to about 4.5 μg/mL, about 0.5 μg/mL to about 4.0 μg/mL, about 0.5 μg/mL to about 3.5 μg/mL, about 0.5 μg/mL to about 3.0 μg/mL, about 0.5 μg/mL to about 2.5 μg/mL, about 0.5 μg/mL to about 2.0 μg/mL, about 0.5 μg/mL to about 1.5 μg/mL, about 0.5 μg/mL to about 1.0 μg/mL, about 1.0 μg/mL to about 4.5 μg/mL, about 1.0 μg/mL to about 4.0 μg/mL, about 1.0 μg/mL to about 3.5 μg/mL, about 1.0 μg/mL to about 3.0 μg/mL, about 1.0 μg/mL to about 2.5 μg/mL, about 1.0 μg/mL to about 2.0 μg/mL, about 1.0 μg/mL to about 1.5 μg/mL, about 1.5 μg/mL to about 4.5 μg/mL, about 1.5 μg/mL to about 4.0 μg/mL, about 1.5 μg/mL to about 3.5 μg/mL, about 1.5 μg/mL to about 3.0 μg/mL, about 1.5 μg/mL to about 2.5 μg/mL, about 1.5 μg/mL to about 2.0 μg/mL, about 2.0 μg/mL to about 4.5 μg/mL, about 2.0 μg/mL to about 4.0 μg/mL, about 2.0 μg/mL to about 3.5 μg/mL, about 2.0 μg/mL to about 3.0 μg/mL, about 2.0 μg/mL to about 2.5 μg/mL, about 2.5 μg/mL to about 4.5 μg/mL, about 2.5 μg/mL to about 4.0 μg/mL, about 2.5 μg/mL to about 3.5 μg/mL, about 2.5 μg/mL to about 3.0 μg/mL, about 3.0 μg/mL to about 4.5 μg/mL, about 3.0 μg/mL to about 4.0 μg/mL, about 3.0 μg/mL to about 3.5 μg/mL, about 3.5 μg/mL to about 4.5 μg/mL, about 3.5 μg/mL to about 4.0 μg/mL, or about 3.5 μg/mL to about 4.5 μg/mL, relative to the pharmaceutical composition.

### Dosage Regimen

In various embodiments, disclosed herein is a treatment regimen. In some embodiments, the treatment regimen includes a dosage pharmaceutical composition with about 100 μg to about 100000 μg of each included active ingredient (i.e., an ETBR antagonist (e.g., a specifically deuterated ETBR antagonist as described herein). The dosage can be a sustained release dosage in which about 50 μg to about 60000 μg of each of the active ingredients is an initial burst, while about 50 μg to about 60000 μg of the each of the active ingredients is a sustained release over 2 hours.

In some embodiments, the administered dosage of the ETBR antagonist (e.g., the specifically deuterated ETBR antagonist), can be about 1.0 μg to about 100000 μg,

In some embodiments, the administered dosage of the ETBR antagonist (e.g., the specifically deuterated ETBR antagonist), can be about 1.0 μg to about 100000 μg, about 1.0 μg to about 90000 μg, about 1.0 μg to about 80000 μg, about 1.0 μg to about 70000 μg, about 1.0 μg to about 60000 μg, about 1.0 μg to about 50000 μg, about 1.0 μg to about 40000 μg, about 1.0 μg to about 30000 μg, about 1.0 μg to about 29000 μg, about 1.0 μg to about 28000 μg, about 1.0 μg to about 27000 μg, about 1.0 μg to about 26000 μg, about 1.0 μg to about 25000 μg, about 1.0 μg to about 24000 μg, about 1.0 μg to about 23000 μg, about 1.0 μg to about 22000 μg, about 1.0 μg to about 21000 μg, about 1.0 μg to about 20000 μg, about 1.0 μg to about 19000 μg, about 1.0 μg to about 18000 μg, about 1.0 μg to about 17000 μg, about 1.0 μg to about 16000 μg, about 1.0 μg to about 15000 μg, about 1.0 μg to about 14000 μg, about 1.0 μg to about 13000 μg, about 1.0 μg to about 12000 μg, about 1.0 μg to about 11000 μg, about 1.0 μg to about 10000 μg, about 1.0 μg to about 9000 μg, about 1.0 μg to about 8000 μg, about 1.0 μg to about 7000 μg, about 1.0 μg to about 6000 μg, about 1.0 μg to about 5000 μg, about 1.0 μg to about 4500 μg, about 1.0 μg to about 4000 μg, about 1.0 μg to about 3500 μg, about 1.0 μg to about 3000 μg, about 1.0 μg to about 2500 μg, about 1.0 μg to about 2000 μg, about 1.0 μg to about 1500 μg, about 1.0 μg to about 1250 μg, about 1.0 μg to about 1000 μg, about 1.0 μg to about 750 μg, about 1.0 μg to about 500 μg, about 1.0 μg to about 300 μg, about 1.0 μg to about 250 μg, about 1.0 μg to about 200 μg, about 1.0 μg to about 150 μg, about 1.0 μg to about 100 μg, about 1.0 μg to about 75 μg, about 1.0 μg to about 50 μg, about 1.0 μg to about 25 μg, about 1.0 μg to about 10 μg, about 10 μg to about 100000 μg, about 10 μg to about 90000 μg, about 10 μg to about 80000 μg, about 10 μg to about 70000 μg, about 10 μg to about 60000 μg, about 10 μg to about 50000 μg, about 10 μg to about 40000 μg, about 10 μg to about 30000 μg, about 10 μg to about 29000 μg, about 10 μg to about 28000 μg, about 10 μg to about 27000 μg, about 10 μg to about 26000 μg, about 10 μg to about 25000 μg, about 10 μg to about 24000 μg, about 10 μg to about 23000 μg, about 10 μg to about 22000 μg, about 10 μg to about 21000 μg, about 10 μg to about 20000 μg, about 10 μg to about 19000 μg, about 10 μg to about 18000 μg, about 10 μg to about 17000 μg, about 10 μg to about 16000 μg, about 10 μg to about 15000 μg, about 10 μg to about 14000 μg, about 10 μg to about 13000 μg, about 10 μg to about 12000 μg, about 10 μg to about 11000 μg, about 10 μg to about 10000 μg, about 10 μg to about 9000 μg, about 10 μg to about 8000 μg, about 10 μg to about 7000 μg, about 10 μg to about 6000 μg, about 10 μg to about 5000 μg, about 10 μg to about 4500 μg, about 10 μg to about 4000 μg, about 10 μg to about 3500 μg, about 10 μg to about 3000 μg, about 10 μg to about 2500 μg, about 10 μg to about 2000 μg, about 10 μg to about 1500 μg, about 10 μg to about 1250 μg, about 10 μg to about 1000 μg, about 10 μg to about 750 μg, about 10 μg to about 500 μg, about 10 μg to about 300 μg, about 10 μg to about 250 μg, about 10 μg to about 200 μg, about 10 μg to about 150 μg, about 10 μg to about 100 μg, about 10 μg to about 75 μg, about 10 μg to about 50 μg, about 10 μg to about 25 μg, about 25 μg to about 100000 μg, about 25 μg to about 90000 μg, about 25 μg to about 80000 μg, about 25 μg to about 70000 μg, about 25 μg to about 60000 μg, about 25 μg to about 50000 μg, about 25 μg to about 40000 μg, about 25 μg to about 30000 μg, about 25 μg to about 29000 μg, about 25 μg to about 28000 μg, about 25 μg to about 27000 μg, about 25 μg to about 26000 μg, about 25 μg to about 25000 μg, about 25 μg to about 24000 μg, about 25 μg to about 23000 μg, about 25 μg to about 22000 μg, about 25 μg to about 21000 μg, about 25 μg to about 20000 μg, about 25 μg to about 19000 μg, about 25 μg to about 18000 μg, about 25 μg to about 17000 μg, about 25 μg to about 16000 μg, about 25 μg to about 15000 μg, about 25 μg to about 14000 μg, about 25 μg to about 13000 μg, about 25 μg to about 12000 μg, about 25 μg to about 11000 μg, about 25 μg to about 10000 μg, about 25 μg to about 9000 μg, about 25 μg to about 8000 μg, about 25 μg to about 7000 μg, about 25 μg to about 6000 μg, about 25 μg to about 5000 μg, about 25 μg to about 4500 μg, about 25 μg to about 4000 μg, about 25 μg to about 3500 μg, about 25 μg to about 3000 μg, about 25 μg to about 2500 μg, about 25 μg to about 2000 μg, about 25 μg to about 1500 μg, about 25 μg to about 1250 μg, about 25 μg to about 1000 μg, about 25 μg to about 750 μg, about 25 μg to about 500 μg, about 25 μg to about 300 μg, about 25 μg to about 250 μg, about 25 μg to about 200 μg, about 25 μg to about 150 μg, about 25 μg to about 100 μg, about 25 μg to about 75 μg, about 25 μg to about 50 μg, about 50 μg to about 100000 μg, about 50 μg to about 90000 μg, about 50 μg to about 80000 μg, about 50 μg to about 70000 μg, about 50 μg to about 60000 μg, about 50 μg to about 50000 μg, about 50 μg to about 40000 μg, about 50 μg to about 30000 μg, about 50 μg to about 29000 μg, about 50 μg to about 28000 μg, about 50 μg to about 27000 μg, about 50 μg to about 26000 μg, about 50 μg to about 25000 μg, about 50 μg to about 24000 μg, about 50 μg to about 23000 μg, about 50 μg to about 22000 μg, about 50 μg to about 21000 μg, about 50 μg to about 20000 μg, about 50 μg to about 19000 μg, about 50 μg to about 18000 μg, about 50 μg to about 17000 μg, about 50 μg to about 16000 μg, about 50 μg to about 15000 μg, about 50 μg to about 14000 μg, about 50 μg to about 13000 μg, about 50 μg to about 12000 μg, about 50 μg to about 11000 μg, about 50 μg to about 10000 μg, about 50 μg to about 9000 μg, about 50 μg to about 8000 μg, about 50 μg to about 7000 μg, about 50 μg to about 6000 μg, about 50 μg to about 5000 μg, about 50 μg to about 4500 μg, about 50 μg to about 4000 μg, about 50 μg to about 3500 μg, about 50 μg to about 3000 μg, about 50 μg to about 2500 μg, about 50 μg to about 2000 μg, about 50 μg to about 1500 μg, about 50 μg to about 1250 μg, about 50 μg to about 1000 μg, about 50 μg to about 750 μg, about 50 μg to about 500 μg, about 50 μg to about 300 μg, about 50 μg to about 250 μg, about 50 μg to about 200 μg, about 50 μg to about 150 μg, about 50 μg to about 100 μg, about 50 μg to about 75 μg, about 75 μg to about 100000 μg, about 75 μg to about 90000 μg, about 75 μg to about 80000 μg, about 75 μg to about 70000 μg, about 75 μg to about 60000 μg, about 75 μg to about 50000 μg, about 75 μg to about 40000 μg, about 75 μg to about 30000 μg, about 75 μg to about 29000 μg, about 75 μg to about 28000 μg, about 75 μg to about 27000 μg, about 75 μg to about 26000 μg, about 75 μg to about 25000 μg, about 75 μg to about 24000 μg, about 75 μg to about 23000 μg, about 75 μg to about 22000 μg, about 75 μg to about 21000 μg, about 75 μg to about 20000 μg, about 75 μg to about 19000 μg, about 75 μg to about 18000 μg, about 75 μg to about 17000 μg, about 75 μg to about 16000 μg, about 75 μg to about 15000 μg, about 75 μg to about 14000 μg, about 75 μg to about 13000 μg, about 75 μg to about 12000 μg, about 75 μg to about 11000 μg, about 75 μg to about 10000 μg, about 75 μg to about 9000 μg, about 75 μg to about 8000 μg, about 75 μg to about 7000 μg, about 75 μg to about 6000 μg, about 75 μg to about 5000 μg, about 75 μg to about 4500 μg, about 75 μg to about 4000 μg, about 75 μg to about 3500 μg, about 75 μg to about 3000 μg, about 75 μg to about 2500 μg, about 75 μg to about 2000 μg, about 75 μg to about 1500 μg, about 75 μg to about 1250 μg, about 75 μg to about 1000 μg, about 75 μg to about 750 μg, about 75 μg to about 500 μg, about 75 μg to about 300 μg, about 75 μg to about 250 μg, about 75 μg to about 200 μg, about 75 μg to about 150 μg, about 75 μg to about 100 μg, about 100 μg to about 100000 μg, about 100 μg to about 90000 μg, about 100 μg to about 80000 μg, about 100 μg to about 70000 μg, about 100 μg to about 60000 μg, about 100 μg to about 50000 μg, about 100 μg to about 40000 μg, about 100 μg to about 30000 μg, about 100 μg to about 29000 μg, about 100 μg to about 28000 μg, about 100 μg to about 27000 μg, about 100 μg to about 26000 μg, about 100 μg to about 25000 μg, about 100 μg to about 24000 μg, about 100 μg to about 23000 μg, about 100 μg to about 22000 μg, about 100 μg to about 21000 μg, about 100 μg to about 20000 μg, about 100 μg to about 19000 μg, about 100 μg to about 18000 μg, about 100 μg to about 17000 μg, about 100 μg to about 16000 μg, about 100 μg to about 15000 μg, about 100 μg to about 14000 μg, about 100 μg to about 13000 μg, about 100 μg to about 12000 μg, about 100 μg to about 11000 μg, about 100 μg to about 10000 μg, about 100 μg to about 9000 μg, about 100 μg to about 8000 μg, about 100 μg to about 7000 μg, about 100 μg to about 6000 μg, about 100 μg to about 5000 μg, about 100 μg to about 4500 μg, about 100 μg to about 4000 μg, about 100 μg to about 3500 μg, about 100 μg to about 3000 μg, about 100 μg to about 2500 μg, about 100 μg to about 2000 μg, about 100 μg to about 1500 μg, about 100 μg to about 1250 μg, about 100 μg to about 1000 μg, about 100 μg to about 750 μg, about 100 μg to about 500 μg, about 100 μg to about 300 μg, about 100 μg to about 250 μg, about 100 μg to about 200 μg, about 100 μg to about 150 μg, about 150 μg to about 100000 μg, about 150 μg to about 90000 μg, about 150 μg to about 80000 μg, about 150 μg to about 70000 μg, about 150 μg to about 60000 μg, about 150 μg to about 50000 μg, about 150 μg to about 40000 μg, about 150 μg to about 30000 μg, about 150 μg to about 29000 μg, about 150 μg to about 28000 μg, about 150 μg to about 27000 μg, about 150 μg to about 26000 μg, about 150 μg to about 25000 μg, about 150 μg to about 24000 μg, about 150 μg to about 23000 μg, about 150 μg to about 22000 μg, about 150 μg to about 21000 μg, about 150 μg to about 20000 μg, about 150 μg to about 19000 μg, about 150 μg to about 18000 μg, about 150 μg to about 17000 μg, about 150 μg to about 16000 μg, about 150 μg to about 15000 μg, about 150 μg to about 14000 μg, about 150 μg to about 13000 μg, about 150 μg to about 12000 μg, about 150 μg to about 11000 μg, about 150 μg to about 10000 μg, about 150 μg to about 9000 μg, about 150 μg to about 8000 μg, about 150 μg to about 7000 μg, about 150 μg to about 6000 μg, about 150 μg to about 5000 μg, about 150 μg to about 4500 μg, about 150 μg to about 4000 μg, about 150 μg to about 3500 μg, about 150 μg to about 3000 μg, about 150 μg to about 2500 μg, about 150 μg to about 2000 μg, about 150 μg to about 1500 μg, about 150 μg to about 1250 μg, about 150 μg to about 1000 μg, about 150 μg to about 750 μg, about 150 μg to about 500 μg, about 150 μg to about 300 μg, about 150 μg to about 250 μg, about 150 μg to about 200 μg, about 250 μg to about 100000 μg, about 250 μg to about 90000 μg, about 250 μg to about 80000 μg, about 250 μg to about 70000 μg, about 250 μg to about 60000 μg, about 250 μg to about 50000 μg, about 250 μg to about 40000 μg, about 250 μg to about 30000 μg, about 250 μg to about 29000 μg, about 250 μg to about 28000 μg, about 250 μg to about 27000 μg, about 250 μg to about 26000 μg, about 250 μg to about 25000 μg, about 250 μg to about 24000 μg, about 250 μg to about 23000 μg, about 250 μg to about 22000 μg, about 250 μg to about 21000 μg, about 250 μg to about 20000 μg, about 250 μg to about 19000 μg, about 250 μg to about 18000 μg, about 250 μg to about 17000 μg, about 250 μg to about 16000 μg, about 250 μg to about 15000 μg, about 250 μg to about 14000 μg, about 250 μg to about 13000 μg, about 250 μg to about 12000 μg, about 250 μg to about 11000 μg, about 250 μg to about 10000 μg, about 250 μg to about 9000 μg, about 250 μg to about 8000 μg, about 250 μg to about 7000 μg, about 250 μg to about 6000 μg, about 250 μg to about 5000 μg, about 250 μg to about 4500 μg, about 250 μg to about 4000 μg, about 250 μg to about 3500 μg, about 250 μg to about 3000 μg, about 250 μg to about 2500 μg, about 250 μg to about 2000 μg, about 250 μg to about 1500 μg, about 250 μg to about 1250 μg, about 250 μg to about 1000 μg, about 250 μg to about 750 μg, about 250 μg to about 500 μg, about 250 μg to about 300 μg, about 300 μg to about 100000 μg, about 300 μg to about 90000 μg, about 300 μg to about 80000 μg, about 300 μg to about 70000 μg, about 300 μg to about 60000 μg, about 300 μg to about 50000 μg, about 300 μg to about 40000 μg, about 300 μg to about 30000 μg, about 300 μg to about 29000 μg, about 300 μg to about 28000 μg, about 300 μg to about 27000 μg, about 300 μg to about 26000 μg, about 300 μg to about 25000 μg, about 300 μg to about 24000 μg, about 300 μg to about 23000 μg, about 300 μg to about 22000 μg, about 300 μg to about 21000 μg, about 300 μg to about 20000 μg, about 300 μg to about 19000 μg, about 300 μg to about 18000 μg, about 300 μg to about 17000 μg, about 300 μg to about 16000 μg, about 300 μg to about 15000 μg, about 300 μg to about 14000 μg, about 300 μg to about 13000 μg, about 300 μg to about 12000 μg, about 300 μg to about 11000 μg, about 300 μg to about 10000 μg, about 300 μg to about 9000 μg, about 300 μg to about 8000 μg, about 300 μg to about 7000 μg, about 300 μg to about 6000 μg, about 300 μg to about 5000 μg, about 300 μg to about 4500 μg, about 300 μg to about 4000 μg, about 300 μg to about 3500 μg, about 300 μg to about 3000 μg, about 300 μg to about 2500 μg, about 300 μg to about 2000 μg, about 300 μg to about 1500 μg, about 300 μg to about 1250 μg, about 300 μg to about 1000 μg, about 300 μg to about 750 μg, about 300 μg to about 500 μg, about 500 μg to about 100000 μg, about 500 μg to about 90000 μg, about 500 μg to about 80000 μg, about 500 μg to about 70000 μg, about 500 μg to about 60000 μg, about 500 μg to about 50000 μg, about 500 μg to about 40000 μg, about 500 μg to about 30000 μg, about 500 μg to about 29000 μg, about 500 μg to about 28000 μg, about 500 μg to about 27000 μg, about 500 μg to about 26000 μg, about 500 μg to about 25000 μg, about 500 μg to about 24000 μg, about 500 μg to about 23000 μg, about 500 μg to about 22000 μg, about 500 μg to about 21000 μg, about 500 μg to about 20000 μg, about 500 μg to about 19000 μg, about 500 μg to about 18000 μg, about 500 μg to about 17000 μg, about 500 μg to about 16000 μg, about 500 μg to about 15000 μg, about 500 μg to about 14000 μg, about 500 μg to about 13000 μg, about 500 μg to about 12000 μg, about 500 μg to about 11000 μg, about 500 μg to about 10000 μg, about 500 μg to about 9000 μg, about 500 μg to about 8000 μg, about 500 μg to about 7000 μg, about 500 μg to about 6000 μg, about 500 μg to about 5000 μg, about 500 μg to about 4500 μg, about 500 μg to about 4000 μg, about 500 μg to about 3500 μg, about 500 μg to about 3000 μg, about 500 μg to about 2500 μg, about 500 μg to about 2000 μg, about 500 μg to about 1500 μg, about 500 μg to about 1250 μg, about 500 μg to about 1000 μg, about 500 μg to about 750 μg, about 750 μg to about 100000 μg, about 750 μg to about 90000 μg, about 750 μg to about 80000 μg, about 750 μg to about 70000 μg, about 750 μg to about 60000 μg, about 750 μg to about 50000 μg, about 750 μg to about 40000 μg, about 750 μg to about 30000 μg, about 750 μg to about 29000 μg, about 750 μg to about 28000 μg, about 750 μg to about 27000 μg, about 750 μg to about 26000 μg, about 750 μg to about 25000 μg, about 750 μg to about 24000 μg, about 750 μg to about 23000 μg, about 750 μg to about 22000 μg, about 750 μg to about 21000 μg, about 750 μg to about 20000 μg, about 750 μg to about 19000 μg, about 750 μg to about 18000 μg, about 750 μg to about 17000 μg, about 750 μg to about 16000 μg, about 750 μg to about 15000 μg, about 750 μg to about 14000 μg, about 750 μg to about 13000 μg, about 750 μg to about 12000 μg, about 750 μg to about 11000 μg, about 750 μg to about 10000 μg, about 750 μg to about 9000 μg, about 750 μg to about 8000 μg, about 750 μg to about 7000 μg, about 750 μg to about 6000 μg, about 750 μg to about 5000 μg, about 750 μg to about 4500 μg, about 750 μg to about 4000 μg, about 750 μg to about 3500 μg, about 750 μg to about 3000 μg, about 750 μg to about 2500 μg, about 750 μg to about 2000 μg, about 750 μg to about 1500 μg, about 750 μg to about 1250 μg, about 750 μg to about 1000 μg, about 1000 μg to about 100000 μg, about 1000 μg to about 90000 μg, about 1000 μg to about 80000 μg, about 1000 μg to about 70000 μg, about 1000 μg to about 60000 μg, about 1000 μg to about 50000 μg, about 1000 μg to about 40000 μg, about 1000 μg to about 30000 μg, about 1000 μg to about 29000 μg, about 1000 μg to about 28000 μg, about 1000 μg to about 27000 μg, about 1000 μg to about 26000 μg, about 1000 μg to about 25000 μg, about 1000 μg to about 24000 μg, about 1000 μg to about 23000 μg, about 1000 μg to about 22000 μg, about 1000 μg to about 21000 μg, about 1000 μg to about 20000 μg, about 1000 μg to about 19000 μg, about 1000 μg to about 18000 μg, about 1000 μg to about 17000 μg, about 1000 μg to about 16000 μg, about 1000 μg to about 15000 μg, about 1000 μg to about 14000 μg, about 1000 μg to about 13000 μg, about 1000 μg to about 12000 μg, about 1000 μg to about 11000 μg, about 1000 μg to about 10000 μg, about 1000 μg to about 9000 μg, about 1000 μg to about 8000 μg, about 1000 μg to about 7000 μg, about 1000 μg to about 6000 μg, about 1000 μg to about 5000 μg, about 1000 μg to about 4500 μg, about 1000 μg to about 4000 μg, about 1000 μg to about 3500 μg, about 1000 μg to about 3000 μg, about 1000 μg to about 2500 μg, about 1000 μg to about 2000 μg, about 1000 μg to about 1500 μg, about 1000 μg to about 1250 μg, about 1250 μg to about 100000 μg, about 1250 μg to about 90000 μg, about 1250 μg to about 80000 μg, about 1250 μg to about 70000 μg, about 1250 μg to about 60000 μg, about 1250 μg to about 50000 μg, about 1250 μg to about 40000 μg, about 1250 μg to about 30000 μg, about 1250 μg to about 29000 μg, about 1250 μg to about 28000 μg, about 1250 μg to about 27000 μg, about 1250 μg to about 26000 μg, about 1250 μg to about 25000 μg, about 1250 μg to about 24000 μg, about 1250 μg to about 23000 μg, about 1250 μg to about 22000 μg, about 1250 μg to about 21000 μg, about 1250 μg to about 20000 μg, about 1250 μg to about 19000 μg, about 1250 μg to about 18000 μg, about 1250 μg to about 17000 μg, about 1250 μg to about 16000 μg, about 1250 μg to about 15000 μg, about 1250 μg to about 14000 μg, about 1250 μg to about 13000 μg, about 1250 μg to about 12000 μg, about 1250 μg to about 11000 μg, about 1250 μg to about 10000 μg, about 1250 μg to about 9000 μg, about 1250 μg to about 8000 μg, about 1250 μg to about 7000 μg, about 1250 μg to about 6000 μg, about 1250 μg to about 5000 μg, about 1250 μg to about 4500 μg, about 1250 μg to about 4000 μg, about 1250 μg to about 3500 μg, about 1250 μg to about 3000 μg, about 1250 μg to about 2500 μg, about 1250 μg to about 2000 μg, about 1250 μg to about 1500 μg, about 1500 μg to about 100000 μg, about 1500 μg to about 90000 μg, about 1500 μg to about 80000 μg, about 1500 μg to about 70000 μg, about 1500 μg to about 60000 μg, about 1500 μg to about 50000 μg, about 1500 μg to about 40000 μg, about 1500 μg to about 30000 μg, about 1500 μg to about 29000 μg, about 1500 μg to about 28000 μg, about 1500 μg to about 27000 μg, about 1500 μg to about 26000 μg, about 1500 μg to about 25000 μg, about 1500 μg to about 24000 μg, about 1500 μg to about 23000 μg, about 1500 μg to about 22000 μg, about 1500 μg to about 21000 μg, about 1500 μg to about 20000 μg, about 1500 μg to about 19000 μg, about 1500 μg to about 18000 μg, about 1500 μg to about 17000 μg, about 1500 μg to about 16000 μg, about 1500 μg to about 15000 μg, about 1500 μg to about 14000 μg, about 1500 μg to about 13000 μg, about 1500 μg to about 12000 μg, about 1500 μg to about 11000 μg, about 1500 μg to about 10000 μg, about 1500 μg to about 9000 μg, about 1500 μg to about 8000 μg, about 1500 μg to about 7000 μg, about 1500 μg to about 6000 μg, about 1500 μg to about 5000 μg, about 1500 μg to about 4500 μg, about 1500 μg to about 4000 μg, about 1500 μg to about 3500 μg, about 1500 μg to about 3000 μg, about 1500 μg to about 2500 μg, about 1500 μg to about 2000 μg, about 2000 μg to about 100000 μg, about 2000 μg to about 90000 μg, about 2000 μg to about 80000 μg, about 2000 μg to about 70000 μg, about 2000 μg to about 60000 μg, about 2000 μg to about 50000 μg, about 2000 μg to about 40000 μg, about 2000 μg to about 30000 μg, about 2000 μg to about 29000 μg, about 2000 μg to about 28000 μg, about 2000 μg to about 27000 μg, about 2000 μg to about 26000 μg, about 2000 μg to about 25000 μg, about 2000 μg to about 24000 μg, about 2000 μg to about 23000 μg, about 2000 μg to about 22000 μg, about 2000 μg to about 21000 μg, about 2000 μg to about 20000 μg, about 2000 μg to about 19000 μg, about 2000 μg to about 18000 μg, about 2000 μg to about 17000 μg, about 2000 μg to about 16000 μg, about 2000 μg to about 15000 μg, about 2000 μg to about 14000 μg, about 2000 μg to about 13000 μg, about 2000 μg to about 12000 μg, about 2000 μg to about 11000 μg, about 2000 μg to about 10000 μg, about 2000 μg to about 9000 μg, about 2000 μg to about 8000 μg, about 2000 μg to about 7000 μg, about 2000 μg to about 6000 μg, about 2000 μg to about 5000 μg, about 2000 μg to about 4500 μg, about 2000 μg to about 4000 μg, about 2000 μg to about 3500 μg, about 2000 μg to about 3000 μg, about 2000 μg to about 2500 μg, about 2500 μg to about 100000 μg, about 2500 μg to about 90000 μg, about 2500 μg to about 80000 μg, about 2500 μg to about 70000 μg, about 2500 μg to about 60000 μg, about 2500 μg to about 50000 μg, about 2500 μg to about 40000 μg, about 2500 μg to about 30000 μg, about 2500 μg to about 29000 μg, about 2500 μg to about 28000 μg, about 2500 μg to about 27000 μg, about 2500 μg to about 26000 μg, about 2500 μg to about 25000 μg, about 2500 μg to about 24000 μg, about 2500 μg to about 23000 μg, about 2500 μg to about 22000 μg, about 2500 μg to about 21000 μg, about 2500 μg to about 20000 μg, about 2500 μg to about 19000 μg, about 2500 μg to about 18000 μg, about 2500 μg to about 17000 μg, about 2500 μg to about 16000 μg, about 2500 μg to about 15000 μg, about 2500 μg to about 14000 μg, about 2500 μg to about 13000 μg, about 2500 μg to about 12000 μg, about 2500 μg to about 11000 μg, about 2500 μg to about 10000 μg, about 2500 μg to about 9000 μg, about 2500 μg to about 8000 μg, about 2500 μg to about 7000 μg, about 2500 μg to about 6000 μg, about 2500 μg to about 5000 μg, about 2500 μg to about 4500 μg, about 2500 μg to about 4000 μg, about 2500 μg to about 3500 μg, about 2500 μg to about 3000 μg, about 3000 μg to about 100000 μg, about 3000 μg to about 90000 μg, about 3000 μg to about 80000 μg, about 3000 μg to about 70000 μg, about 3000 μg to about 60000 μg, about 3000 μg to about 50000 μg, about 3000 μg to about 40000 μg, about 3000 μg to about 30000 μg, about 3000 μg to about 29000 μg, about 3000 μg to about 28000 μg, about 3000 μg to about 27000 μg, about 3000 μg to about 26000 μg, about 3000 μg to about 25000 μg, about 3000 μg to about 24000 μg, about 3000 μg to about 23000 μg, about 3000 μg to about 22000 μg, about 3000 μg to about 21000 μg, about 3000 μg to about 20000 μg, about 3000 μg to about 19000 μg, about 3000 μg to about 18000 μg, about 3000 μg to about 17000 μg, about 3000 μg to about 16000 μg, about 3000 μg to about 15000 μg, about 3000 μg to about 14000 μg, about 3000 μg to about 13000 μg, about 3000 μg to about 12000 μg, about 3000 μg to about 11000 μg, about 3000 μg to about 10000 μg, about 3000 μg to about 9000 μg, about 3000 μg to about 8000 μg, about 3000 μg to about 7000 μg, about 3000 μg to about 6000 μg, about 3000 μg to about 5000 μg, about 3000 μg to about 4500 μg, about 3000 μg to about 4000 μg, about 3500 μg to about 100000 μg, about 3500 μg to about 90000 μg, about 3500 μg to about 80000 μg, about 3500 μg to about 70000 μg, about 3500 μg to about 60000 μg, about 3500 μg to about 50000 μg, about 3500 μg to about 40000 μg, about 3500 μg to about 30000 μg, about 3500 μg to about 29000 μg, about 3500 μg to about 28000 μg, about 3500 μg to about 27000 μg, about 3500 μg to about 26000 μg, about 3500 μg to about 25000 μg, about 3500 μg to about 24000 μg, about 3500 μg to about 23000 μg, about 3500 μg to about 22000 μg, about 3500 μg to about 21000 μg, about 3500 μg to about 20000 μg, about 3500 μg to about 19000 μg, about 3500 μg to about 18000 μg, about 3500 μg to about 17000 μg, about 3500 μg to about 16000 μg, about 3500 μg to about 15000 μg, about 3500 μg to about 14000 μg, about 3500 μg to about 13000 μg, about 3500 μg to about 12000 μg, about 3500 μg to about 11000 μg, about 3500 μg to about 10000 μg, about 3500 μg to about 9000 μg, about 3500 μg to about 8000 μg, about 3500 μg to about 7000 μg, about 3500 μg to about 6000 μg about 3500 μg to about 5000 μg, about 3500 μg to about 4500 μg, about 3500 μg to about 4000 μg, about 4000 μg to about 100000 μg, about 4000 μg to about 90000 μg, about 4000 μg to about 80000 μg, about 4000 μg to about 70000 μg, about 4000 μg to about 60000 μg, about 4000 μg to about 50000 μg, about 4000 μg to about 40000 μg, about 4000 μg to about 30000 μg, about 4000 μg to about 29000 μg, about 4000 μg to about 28000 μg, about 4000 μg to about 27000 μg, about 4000 μg to about 26000 μg, about 4000 μg to about 25000 μg, about 4000 μg to about 24000 μg, about 4000 μg to about 23000 μg, about 4000 μg to about 22000 μg, about 4000 μg to about 21000 μg, about 4000 μg to about 20000 μg, about 4000 μg to about 19000 μg, about 4000 μg to about 18000 μg, about 4000 μg to about 17000 μg, about 4000 μg to about 16000 μg, about 4000 μg to about 15000 μg, about 4000 μg to about 14000 μg, about 4000 μg to about 13000 μg, about 4000 μg to about 12000 μg, about 4000 μg to about 11000 μg, about 4000 μg to about 10000 μg, about 4000 μg to about 9000 μg, about 4000 μg to about 8000 μg, about 4000 μg to about 7000 μg, about 4000 μg to about 6000 μg, about 4000 μg to about 5000 μg, about 4000 μg to about 4500 μg, about 4500 μg to about 100000 μg, about 4500 μg to about 90000 μg, about 4500 μg to about 80000 μg, about 4500 μg to about 70000 μg, about 4500 μg to about 60000 μg, about 4500 μg to about 50000 μg, about 4500 μg to about 40000 μg, about 4500 μg to about 30000 μg, about 4500 μg to about 29000 μg, about 4500 μg to about 28000 μg, about 4500 μg to about 27000 μg, about 4500 μg to about 26000 μg, about 4500 μg to about 25000 μg, about 4500 μg to about 24000 μg, about 4500 μg to about 23000 μg, about 4500 μg to about 22000 μg, about 4500 μg to about 21000 μg, about 4500 μg to about 20000 μg, about 4500 μg to about 19000 μg, about 4500 μg to about 18000 μg, about 4500 μg to about 17000 μg, about 4500 μg to about 16000 μg, about 4500 μg to about 15000 μg, about 4500 μg to about 14000 μg, about 4500 μg to about 13000 μg, about 4500 μg to about 12000 μg, about 4500 μg to about 11000 μg, about 4500 μg to about 10000 μg, about 4500 μg to about 9000 μg, about 4500 μg to about 8000 μg, about 4500 μg to about 7000 μg, about 4500 μg to about 6000 μg, about 4500 μg to about 5000 μg, about 5000 μg to about 100000 μg, about 5000 μg to about 90000 μg, about 5000 μg to about 80000 μg, about 5000 μg to about 70000 μg, about 5000 μg to about 60000 μg, about 5000 μg to about 50000 μg, about 5000 μg to about 40000 μg, about 5000 μg to about 30000 μg, about 5000 μg to about 29000 μg, about 5000 μg to about 28000 μg, about 5000 μg to about 27000 μg, about 5000 μg to about 26000 μg, about 5000 μg to about 25000 μg, about 5000 μg to about 24000 μg, about 5000 μg to about 23000 μg, about 5000 μg to about 22000 μg, about 5000 μg to about 21000 μg, about 5000 μg to about 20000 μg, about 5000 μg to about 19000 μg, about 5000 μg to about 18000 μg, about 5000 μg to about 17000 μg, about 5000 μg to about 16000 μg, about 5000 μg to about 15000 μg, about 5000 μg to about 14000 μg, about 5000 μg to about 13000 μg, about 5000 μg to about 12000 μg, about 5000 μg to about 11000 μg, about 5000 μg to about 10000 μg, about 5000 μg to about 9000 μg, about 5000 μg to about 8000 μg, about 5000 μg to about 7000 μg, about 5000 μg to about 6000 μg, about 6000 μg to about 100000 μg, about 6000 μg to about 90000 μg, about 6000 μg to about 80000 μg, about 6000 μg to about 70000 μg, about 6000 μg to about 60000 μg, about 6000 μg to about 50000 μg, about 6000 μg to about 40000 μg, about 6000 μg to about 30000 μg, about 6000 μg to about 29000 μg, about 6000 μg to about 28000 μg, about 6000 μg to about 27000 μg, about 6000 μg to about 26000 μg, about 6000 μg to about 25000 μg, about 6000 μg to about 24000 μg, about 6000 μg to about 23000 μg, about 6000 μg to about 22000 μg, about 6000 μg to about 21000 μg, about 6000 μg to about 20000 μg, about 6000 μg to about 19000 μg, about 6000 μg to about 18000 μg, about 6000 μg to about 17000 μg, about 6000 μg to about 16000 μg, about 6000 μg to about 15000 μg, about 6000 μg to about 14000 μg, about 6000 μg to about 13000 μg, about 6000 μg to about 12000 μg, about 6000 μg to about 11000 μg, about 6000 μg to about 10000 μg, about 6000 μg to about 9000 μg, about 6000 μg to about 8000 μg, about 6000 μg to about 7000 μg, about 7000 μg to about 100000 μg, about 7000 μg to about 90000 μg, about 7000 μg to about 80000 μg, about 7000 μg to about 70000 μg, about 7000 μg to about 60000 μg, about 7000 μg to about 50000 μg, about 7000 μg to about 40000 μg, about 7000 μg to about 30000 μg, about 7000 μg to about 29000 μg, about 7000 μg to about 28000 μg, about 7000 μg to about 27000 μg, about 7000 μg to about 26000 μg, about 7000 μg to about 25000 μg, about 7000 μg to about 24000 μg, about 7000 μg to about 23000 μg, about 7000 μg to about 22000 μg, about 7000 μg to about 21000 μg, about 7000 μg to about 20000 μg, about 7000 μg to about 19000 μg, about 7000 μg to about 18000 μg, about 7000 μg to about 17000 μg, about 7000 μg to about 16000 μg, about 7000 μg to about 15000 μg, about 7000 μg to about 14000 μg, about 7000 μg to about 13000 μg, about 7000 μg to about 12000 μg, about 7000 μg to about 11000 μg, about 7000 μg to about 10000 μg, about 7000 μg to about 9000 μg, about 7000 μg to about 8000 μg, about 8000 μg to about 100000 μg, about 8000 μg to about 90000 μg, about 8000 μg to about 80000 μg, about 8000 μg to about 70000 μg, about 8000 μg to about 60000 μg, about 8000 μg to about 50000 μg, about 8000 μg to about 40000 μg, about 8000 μg to about 30000 μg, about 8000 μg to about 29000 μg, about 8000 μg to about 28000 μg, about 8000 μg to about 27000 μg, about 8000 μg to about 26000 μg, about 8000 μg to about 25000 μg, about 8000 μg to about 24000 μg, about 8000 μg to about 23000 μg, about 8000 μg to about 22000 μg, about 8000 μg to about 21000 μg, about 8000 μg to about 20000 μg, about 8000 μg to about 19000 μg, about 8000 μg to about 18000 μg, about 8000 μg to about 17000 μg, about 8000 μg to about 16000 μg, about 8000 μg to about 15000 μg, about 8000 μg to about 14000 μg, about 8000 μg to about 13000 μg, about 8000 μg to about 12000 μg, about 8000 μg to about 11000 μg, about 8000 μg to about 10000 μg, about 8000 μg to about 9000 μg, about 9000 μg to about 100000 μg, about 9000 μg to about 90000 μg, about 9000 μg to about 80000 μg, about 9000 μg to about 70000 μg, about 9000 μg to about 60000 μg, about 9000 μg to about 50000 μg, about 9000 μg to about 40000 μg, about 9000 μg to about 30000 μg, about 9000 μg to about 29000 μg, about 9000 μg to about 28000 μg, about 9000 μg to about 27000 μg, about 9000 μg to about 26000 μg, about 9000 μg to about 25000 μg, about 9000 μg to about 24000 μg, about 9000 μg to about 23000 μg, about 9000 μg to about 22000 μg, about 9000 μg to about 21000 μg, about 9000 μg to about 20000 μg, about 9000 μg to about 19000 μg, about 9000 μg to about 18000 μg, about 9000 μg to about 17000 μg, about 9000 μg to about 16000 μg, about 9000 μg to about 15000 μg, about 9000 μg to about 14000 μg, about 9000 μg to about 13000 μg, about 9000 μg to about 12000 μg, about 9000 μg to about 11000 μg, about 9000 μg to about 10000 μg, about 10000 μg to about 100000 μg, about 10000 μg to about 90000 μg, about 10000 μg to about 80000 μg, about 10000 μg to about 70000 μg, about 10000 μg to about 60000 μg, about 10000 μg to about 50000 μg, about 10000 μg to about 40000 μg, about 10000 μg to about 30000 μg, about 10000 μg to about 29000 μg, about 10000 μg to about 28000 μg, about 10000 μg to about 27000 μg, about 10000 μg to about 26000 μg, about 10000 μg to about 25000 μg, about 10000 μg to about 24000 μg, about 10000 μg to about 23000 μg, about 10000 μg to about 22000 μg, about 10000 μg to about 21000 μg, about 10000 μg to about 20000 μg, about 10000 μg to about 19000 μg, about 10000 μg to about 18000 μg, about 10000 μg to about 17000 μg, about 10000 μg to about 16000 μg, about 10000 μg to about 15000 μg, about 10000 μg to about 14000 μg, about 10000 μg to about 13000 μg, about 10000 μg to about 12000 μg, about 10000 μg to about 11000 μg, about 11000 μg to about 100000 μg, about 11000 μg to about 90000 μg, about 11000 μg to about 80000 μg, about 11000 μg to about 70000 μg, about 11000 μg to about 60000 μg, about 11000 μg to about 50000 μg, about 11000 μg to about 40000 μg, about 11000 μg to about 30000 μg, about 11000 μg to about 29000 μg, about 11000 μg to about 28000 μg, about 11000 μg to about 27000 μg, about 11000 μg to about 26000 μg, about 11000 μg to about 25000 μg, about 11000 μg to about 24000 μg, about 11000 μg to about 23000 μg, about 11000 μg to about 22000 μg, about 11000 μg to about 21000 μg, about 11000 μg to about 20000 μg, about 11000 μg to about 19000 μg, about 11000 μg to about 18000 μg, about 11000 μg to about 17000 μg, about 11000 μg to about 16000 μg, about 11000 μg to about 15000 μg, about 11000 μg to about 14000 μg, about 11000 μg to about 13000 μg, about 11000 μg to about 12000 μg, about 12000 μg to about 100000 μg, about 12000 μg to about 90000 μg, about 12000 μg to about 80000 μg, about 12000 μg to about 70000 μg, about 12000 μg to about 60000 μg, about 12000 μg to about 50000 μg, about 12000 μg to about 40000 μg, about 12000 μg to about 30000 μg, about 12000 μg to about 29000 μg, about 12000 μg to about 28000 μg, about 12000 μg to about 27000 μg, about 12000 μg to about 26000 μg, about 12000 μg to about 25000 μg, about 12000 μg to about 24000 μg, about 12000 μg to about 23000 μg, about 12000 μg to about 22000 μg, about 12000 μg to about 21000 μg, about 12000 μg to about 20000 μg, about 12000 μg to about 19000 μg, about 12000 μg to about 18000 μg, about 12000 μg to about 17000 μg, about 12000 μg to about 16000 μg, about 12000 μg to about 15000 μg, about 12000 μg to about 14000 μg, about 12000 μg to about 13000 μg, about 13000 μg to about 100000 μg, about 13000 μg to about 90000 μg, about 13000 μg to about 80000 μg, about 13000 μg to about 70000 μg, about 13000 μg to about 60000 μg, about 13000 μg to about 50000 μg, about 13000 μg to about 40000 μg, about 13000 μg to about 30000 μg, about 13000 μg to about 29000 μg, about 13000 μg to about 28000 μg, about 13000 μg to about 27000 μg, about 13000 μg to about 26000 μg, about 13000 μg to about 25000 μg, about 13000 μg to about 24000 μg, about 13000 μg to about 23000 μg, about 13000 μg to about 22000 μg, about 13000 μg to about 21000 μg, about 13000 μg to about 20000 μg, about 13000 μg to about 19000 μg, about 13000 μg to about 18000 μg, about 13000 μg to about 17000 μg, about 13000 μg to about 16000 μg, about 13000 μg to about 15000 μg, about 13000 μg to about 14000 μg, about 14000 μg to about 100000 μg, about 14000 μg to about 90000 μg, about 14000 μg to about 80000 μg, about 14000 μg to about 70000 μg, about 14000 μg to about 60000 μg, about 14000 μg to about 50000 μg, about 14000 μg to about 40000 μg, about 14000 μg to about 30000 μg, about 14000 μg to about 29000 μg, about 14000 μg to about 28000 μg, about 14000 μg to about 27000 μg, about 14000 μg to about 26000 μg, about 14000 μg to about 25000 μg, about 14000 μg to about 24000 μg, about 14000 μg to about 23000 μg, about 14000 μg to about 22000 μg, about 14000 μg to about 21000 μg, about 14000 μg to about 20000 μg, about 14000 μg to about 19000 μg, about 14000 μg to about 18000 μg, about 14000 μg to about 17000 μg, about 14000 μg to about 16000 μg, about 14000 μg to about 15000 μg, about 15000 μg to about 100000 μg, about 15000 μg to about 90000 μg, about 15000 μg to about 80000 μg, about 15000 μg to about 70000 μg, about 15000 μg to about 60000 μg, about 15000 μg to about 50000 μg, about 15000 μg to about 40000 μg, about 15000 μg to about 30000 μg, about 15000 μg to about 29000 μg, about 15000 μg to about 28000 μg, about 15000 μg to about 27000 μg, about 15000 μg to about 26000 μg, about 15000 μg to about 25000 μg, about 15000 μg to about 24000 μg, about 15000 μg to about 23000 μg, about 15000 μg to about 22000 μg, about 15000 μg to about 21000 μg, about 15000 μg to about 20000 μg, about 15000 μg to about 19000 μg, about 15000 μg to about 18000 μg, about 15000 μg to about 17000 μg, about 15000 μg to about 16000 μg, about 16000 μg to about 100000 μg, about 16000 μg to about 90000 μg, about 16000 μg to about 80000 μg, about 16000 μg to about 70000 μg, about 16000 μg to about 60000 μg, about 16000 μg to about 50000 μg, about 16000 μg to about 40000 μg, about 16000 μg to about 30000 μg, about 16000 μg to about 29000 μg, about 16000 μg to about 28000 μg, about 16000 μg to about 27000 μg, about 16000 μg to about 26000 μg, about 16000 μg to about 25000 μg, about 16000 μg to about 24000 μg, about 16000 μg to about 23000 μg, about 16000 μg to about 22000 μg, about 16000 μg to about 21000 μg, about 16000 μg to about 20000 μg, about 16000 μg to about 19000 μg, about 16000 μg to about 18000 μg, about 16000 μg to about 17000 μg, about 17000 μg to about 100000 μg, about 17000 μg to about 90000 μg, about 17000 μg to about 80000 μg, about 17000 μg to about 70000 μg, about 17000 μg to about 60000 μg, about 17000 μg to about 50000 μg, about 17000 μg to about 40000 μg, about 17000 μg to about 30000 μg, about 17000 μg to about 29000 μg, about 17000 μg to about 28000 μg, about 17000 μg to about 27000 μg, about 17000 μg to about 26000 μg, about 17000 μg to about 25000 μg, about 17000 μg to about 24000 μg, about 17000 μg to about 23000 μg, about 17000 μg to about 22000 μg, about 17000 μg to about 21000 μg, about 17000 μg to about 20000 μg, about 17000 μg to about 19000 μg, about 17000 μg to about 18000 μg, about 18000 μg to about 100000 μg, about 18000 μg to about 90000 μg, about 18000 μg to about 80000 μg, about 18000 μg to about 70000 μg, about 18000 μg to about 60000 μg, about 18000 μg to about 50000 μg, about 18000 μg to about 40000 μg, about 18000 μg to about 30000 μg, about 18000 μg to about 29000 μg, about 18000 μg to about 28000 μg, about 18000 μg to about 27000 μg, about 18000 μg to about 26000 μg, about 18000 μg to about 25000 μg, about 18000 μg to about 24000 μg, about 18000 μg to about 23000 μg, about 18000 μg to about 22000 μg, about 18000 μg to about 21000 μg, about 18000 μg to about 20000 μg, about 18000 μg to about 19000 μg, about 19000 μg to about 100000 μg, about 19000 μg to about 90000 μg, about 19000 μg to about 80000 μg, about 19000 μg to about 70000 μg, about 19000 μg to about 60000 μg, about 19000 μg to about 50000 μg, about 19000 μg to about 40000 μg, about 19000 μg to about 30000 μg, about 19000 μg to about 29000 μg, about 19000 μg to about 28000 μg, about 19000 μg to about 27000 μg, about 19000 μg to about 26000 μg, about 19000 μg to about 25000 μg, about 19000 μg to about 24000 μg, about 19000 μg to about 23000 μg, about 19000 μg to about 22000 μg, about 19000 μg to about 21000 μg, about 19000 μg to about 20000 μg, about 20000 μg to about 100000 μg, about 20000 μg to about 90000 μg, about 20000 μg to about 80000 μg, about 20000 μg to about 70000 μg, about 20000 μg to about 60000 μg, about 20000 μg to about 50000 μg, about 20000 μg to about 40000 μg, about 20000 μg to about 30000 μg, about 20000 μg to about 29000 μg, about 20000 μg to about 28000 μg, about 20000 μg to about 27000 μg, about 20000 μg to about 26000 μg, about 20000 μg to about 25000 μg, about 20000 μg to about 24000 μg, about 20000 μg to about 23000 μg, about 20000 μg to about 22000 μg, about 20000 μg to about 21000 μg, about 21000 μg to about 100000 μg, about 21000 μg to about 90000 μg, about 21000 μg to about 80000 μg, about 21000 μg to about 70000 μg, about 21000 μg to about 60000 μg, about 21000 μg to about 50000 μg, about 21000 μg to about 40000 μg, about 21000 μg to about 30000 μg, about 21000 μg to about 29000 μg, about 21000 μg to about 28000 μg, about 21000 μg to about 27000 μg, about 21000 μg to about 26000 μg, about 21000 μg to about 25000 μg, about 21000 μg to about 24000 μg, about 21000 μg to about 23000 μg, about 21000 μg to about 22000 μg, about 22000 μg to about 100000 μg, about 22000 μg to about 90000 μg, about 22000 μg to about 80000 μg, about 22000 μg to about 70000 μg, about 22000 μg to about 60000 μg, about 22000 μg to about 50000 μg, about 22000 μg to about 40000 μg, about 22000 μg to about 30000 μg, about 22000 μg to about 29000 μg, about 22000 μg to about 28000 μg, about 22000 μg to about 27000 μg, about 22000 μg to about 26000 μg, about 22000 μg to about 25000 μg, about 22000 μg to about 24000 μg, about 22000 μg to about 23000 μg, about 23000 μg to about 100000 μg, about 23000 μg to about 90000 μg, about 23000 μg to about 80000 μg, about 23000 μg to about 70000 μg, about 23000 μg to about 60000 μg, about 23000 μg to about 50000 μg, about 23000 μg to about 40000 μg, about 23000 μg to about 30000 μg, about 23000 μg to about 29000 μg, about 23000 μg to about 28000 μg, about 23000 μg to about 27000 μg, about 23000 μg to about 26000 μg, about 23000 μg to about 25000 μg, about 23000 μg to about 24000 μg, about 24000 μg to about 100000 μg, about 24000 μg to about 90000 μg, about 24000 μg to about 80000 μg, about 24000 μg to about 70000 μg, about 24000 μg to about 60000 μg, about 24000 μg to about 50000 μg, about 24000 μg to about 40000 μg, about 24000 μg to about 30000 μg, about 24000 μg to about 29000 μg, about 24000 μg to about 28000 μg, about 24000 μg to about 27000 μg, about 24000 μg to about 26000 μg, about 24000 μg to about 25000 μg, about 25000 μg to about 100000 μg, about 25000 μg to about 90000 μg, about 25000 μg to about 80000 μg, about 25000 μg to about 70000 μg, about 25000 μg to about 60000 μg, about 25000 μg to about 50000 μg, about 25000 μg to about 40000 μg, about 25000 μg to about 30000 μg, about 25000 μg to about 29000 μg, about 25000 μg to about 28000 μg, about 25000 μg to about 27000 μg, about 25000 μg to about 26000 μg, about 26000 μg to about 100000 μg, about 26000 μg to about 90000 μg, about 26000 μg to about 80000 μg, about 26000 μg to about 70000 μg, about 26000 μg to about 60000 μg, about 26000 μg to about 50000 μg, about 26000 μg to about 40000 μg, about 26000 μg to about 30000 μg, about 26000 μg to about 29000 μg, about 26000 μg to about 28000 μg, about 26000 μg to about 27000 μg, about 27000 μg to about 100000 μg, about 27000 μg to about 90000 μg, about 27000 μg to about 80000 μg, about 27000 μg to about 70000 μg, about 27000 μg to about 60000 μg, about 27000 μg to about 50000 μg, about 27000 μg to about 40000 μg, about 27000 μg to about 30000 μg, about 27000 μg to about 29000 μg, about 27000 μg to about 28000 μg, about 28000 μg to about 100000 μg, about 28000 μg to about 90000 μg, about 28000 μg to about 80000 μg, about 28000 μg to about 70000 μg, about 28000 μg to about 60000 μg, about 28000 μg to about 50000 μg, about 28000 μg to about 40000 μg, about 28000 μg to about 30000 μg, about 28000 μg to about 29000 μg, about 29000 μg to about 100000 μg, about 29000 μg to about 90000 μg, about 29000 μg to about 80000 μg, about 29000 μg to about 70000 μg, about 29000 μg to about 60000 μg, about 29000 μg to about 50000 μg, about 29000 μg to about 40000 μg, about 29000 μg to about 30000 μg, about 30000 μg to about 100000 μg, about 30000 μg to about 90000 μg, about 30000 μg to about 80000 μg, about 30000 μg to about 70000 μg, about 30000 μg to about 60000 μg, about 30000 μg to about 50000 μg, about 30000 μg to about 40000 μg, about 40000 μg to about 100000 μg, about 40000 μg to about 90000 μg, about 40000 μg to about 80000 μg, about 40000 μg to about 70000 μg, about 40000 μg to about 60000 μg, about 40000 μg to about 50000 μg, about 50000 μg to about 100000 μg, about 50000 μg to about 90000 μg, about 50000 μg to about 80000 μg, about 50000 μg to about 70000 μg, about 50000 μg to about 60000 μg, about 60000 μg to about 100000 μg, about 60000 μg to about 90000 μg, about 60000 μg to about 80000 μg, about 60000 μg to about 70000 μg, about 70000 μg to about 100000 μg, about 70000 μg to about 90000 μg, about 70000 μg to about 80000 μg, about 80000 μg to about 100000 μg, about 80000 μg to about 90000 μg and about 90000 μg to about 100000.

In some embodiments, the administered dosage of the ETBR antagonist (e.g., the specifically deuterated ETBR antagonist), can be about 1.0 μg to about 10000 μg, about 1.0 μg to about 5000 μg, about 1.0 μg to about 2500 μg, about 1.0 μg to about 1000 μg, about 1.0 μg to about 500 μg, about 1.0 μg to about 250 μg, about 1.0 μg to about 100 μg, about 1.0 μg to about 75 μg, about 1.0 μg to about 50 μg, about 1.0 μg to about 25 μg, about 1.0 μg to about 10 μg, about 10 μg to about 10000 μg, about 10 μg to about 5000 μg, about 10 μg to about 2500 μg, about 10 μg to about 1000 μg, about 10 μg to about 500 μg, about 10 μg to about 250 μg, about 10 μg to about 100 μg, about 10 μg to about 75 μg, about 10 μg to about 50 μg, about 50 μg to about 10000 μg, about 50 μg to about 5000 μg, about 50 μg to about 2500 μg, about 50 μg to about 2000 μg, about 50 μg to about 1000 μg, about 50 μg to about 750 μg, about 50 μg to about 500 μg, about 50 μg to about 250 μg, about 50 μg to about 200 μg, about 50 μg to about 100 μg, about 100 μg to about 10000 μg, about 100 μg to about 5000 μg, about 100 μg to about 2500 μg, about 100 μg to about 2000 μg, about 100 μg to about 1500 μg, about 100 μg to about 1250 μg, about 100 μg to about 1000 μg, about 100 μg to about 750 μg, about 100 μg to about 500 μg, about 100 μg to about 250 μg, about 100 μg to about 200 μg, about 250 μg to about 10000 μg, about 250 μg to about 5000 μg, about 250 μg to about 2500 μg, about 250 μg to about 2000 μg, about 250 μg to about 1500 μg, about 250 μg to about 1250 μg, about 250 μg to about 1000 μg, about 250 μg to about 750 μg, about 250 μg to about 500 μg, about 500 μg to about 10000 μg, about 500 μg to about 7000 μg, about 500 μg to about 5000 μg, about 500 μg to about 3000 μg, about 500 μg to about 2500 μg, about 500 μg to about 2000 μg, about 500 μg to about 1500 μg, about 500 μg to about 1250 μg, about 500 μg to about 1000 μg, about 500 μg to about 750 μg, about 750 μg to about 20000 μg, about 750 μg to about 15000 μg, about 750 μg to about 10000 μg, about 750 μg to about 7000 μg, about 750 μg to about 5000 μg, about 750 μg to about 3000 μg, about 750 μg to about 2500 μg, about 750 μg to IPTS/128945731.1_53 about 2000 μg, about 750 μg to about 1500 μg, about 750 μg to about 1250 μg, about 750 μg to about 1000 μg, about 1000 μg to about 25000 μg, about 1000 μg to about 20000 μg, about 1000 μg to about 15000 μg, about 1000 μg to about 10000 μg, about 1000 μg to about 7000 μg, about 1000 μg to about 5000 μg, about 1000 μg to about 3000 μg, about 1000 μg to about 2500 μg, about 1000 μg to about 2000 μg, about 1000 μg to about 1500 μg, about 1500 μg to about 25000 μg, about 1500 μg to about 20000 μg, about 1500 μg to about 15000 μg, about 1500 μg to about 10000 μg, about 1500 μg to about 7000 μg, about 1500 μg to about 5000 μg, about 1500 μg to about 3000 μg, about 1500 μg to about 2500 μg, about 1500 μg to about 2000 μg, about 2000 μg to about 30000 μg, about 2000 μg to about 25000 μg, about 2000 μg to about 20000 μg, about 2000 μg to about 15000 μg, about 2000 μg to about 10000 μg, about 2000 μg to about 7000 μg, about 2000 μg to about 5000 μg, about 2000 μg to about 4000 μg, about 2000 μg to about 3000 μg, about 2000 μg to about 2500 μg, about 2500 μg to about 30000 μg, about 2500 μg to about 25000 μg, about 2500 μg to about 20000 μg, about 2500 μg to about 15000 μg, about 2500 μg to about 12000 μg, about 2500 μg to about 10000 μg, about 2500 μg to about 7000 μg, about 2500 μg to about 5000 μg, about 2500 μg to about 4000 μg, about 2500 μg to about 3000 μg, about 3000 μg to about 50000 μg, about 3000 μg to about 25000 μg, about 3000 μg to about 20000 μg, about 3000 μg to about 15000 μg, about 3000 μg to about 12000 μg, about 3000 μg to about 10000 μg, about 3000 μg to about 7000 μg, about 3000 μg to about 6000 μg, about 3000 μg to about 5000 μg, about 3000 μg to about 4000 μg, about 4000 μg to about 50000 μg, about 4000 μg to about 30000 μg, about 4000 μg to about 25000 μg, about 4000 μg to about 20000 μg, about 4000 μg to about 15000 μg, about 4000 μg to about 12000 μg, about 4000 μg to about 10000 μg, about 4000 μg to about 8000 μg, about 4000 μg to about 6000 μg, about 4000 μg to about 5000 μg, about 5000 μg to about 50000 μg, about 5000 μg to about 40000 μg, about 5000 μg to about 30000 μg, about 5000 μg to about 25000 μg, about 5000 μg to about 20000 μg, about 5000 μg to about 15000 μg, about 5000 μg to about 12000 μg, about 5000 μg to about 10000 μg, about 5000 μg to about 8000 μg, about 5000 μg to about 7000 μg, about 5000 μg to about 6000 μg, about 6000 μg to about 50000 μg, about 6000 μg to about 30000 μg, about 6000 μg to about 25000 μg, about 6000 μg to about 20000 μg, about 6000 μg to about 17000 μg, about 6000 μg to about 15000 μg, about 6000 μg to about 12000 μg, about 6000 μg to about 10000 μg, about 6000 μg to about 9000 μg, about 6000 μg to about 8000 μg, about 6000 μg to about 7000 μg, about 7000 μg to about 70000 μg, about 7000 μg to about 50000 μg, about 7000 μg to about 40000 μg, about 7000 μg to about 30000 μg, about 7000 μg to about 25000 μg, about 7000 μg to about 20000 μg, about 7000 μg to about 17000 μg, about 7000 μg to about 15000 μg, about 7000 μg to about 12000 μg, about 7000 μg to about 10000 μg, about 7000 μg to about 8000 μg, about 8000 μg to about 80000 μg, about 8000 μg to about 60000 μg, about 8000 μg to about 50000 μg, about 8000 μg to about 30000 μg, about 8000 μg to about 25000 μg, about 8000 μg to about 20000 μg, about 8000 μg to about 17000 μg, about 8000 μg to about 15000 μg, about 8000 μg to about 12000 μg, about 8000 μg to about 10000 μg, about 10000 μg to about 100000 μg, about 10000 μg to about 80000 μg, about 10000 μg to about 70000 μg, about 10000 μg to about 60000 μg, about 10000 μg to about 50000 μg, about 10000 μg to about 40000 μg, about 10000 μg to about 30000 μg, about 10000 μg to about 25000 μg, about 10000 μg to about 20000 μg, 10000 μg to about 18000 μg, about 10000 μg to about 16000 μg, about 10000 μg to about 15000 μg, about 10000 μg to about 12000 μg, about 12000 μg to about 100000 μg, about 12000 μg to about 90000 μg, about 12000 μg to about 80000 μg, about 12000 μg to about 70000 μg, about 12000 μg to about 60000 μg, about 12000 μg to about 50000 μg, about 12000 μg to about 40000 μg, about 12000 μg to about 30000 μg, about 12000 μg to about 25000 μg, about 12000 μg to about 20000 μg, about 12000 μg to about 18000 μg, about 12000 μg to about 16000 μg, about 12000 μg to about 15000 μg, about 12000 μg to about 14000 μg, about 15000 μg to about 100000 μg, about 15000 μg to about 90000 μg, about 15000 μg to about 80000 μg, about 15000 μg to about 70000 μg, about 15000 μg to about 60000 μg, about 15000 μg to about 50000 μg, about 15000 μg to about 40000 μg, about 15000 μg to about 30000 μg, about 15000 μg to about 25000 μg, about 15000 μg to about 20000 μg, about 20000 μg to about 100000 μg, about 20000 μg to about 90000 μg, about 20000 μg to about 80000 μg, about 20000 μg to about 70000 μg, about 20000 μg to about 60000 μg, about 20000 μg to about 50000 μg, about 20000 μg to about 40000 μg, about 20000 μg to about 30000 μg, about 20000 μg to about 25000 μg, about 25000 μg to about 100000 μg, about 25000 μg to about 90000 μg, about 25000 μg to about 80000 μg, about 25000 μg to about 70000 μg, about 25000 μg to about 60000 μg, about 25000 μg to about 50000 μg, about 25000 μg to about 40000 μg, about 25000 μg to about 30000 μg, about 30000 μg to about 100000 μg, about 30000 μg to about 90000 μg, about 30000 μg to about 80000 μg, about 30000 μg to about 70000 μg, about 30000 μg to about 60000 μg, about 30000 μg to about 50000 μg, about 30000 μg to about 40000 μg, about 40000 μg to about 100000 μg, about 40000 μg to about 80000 μg, about 40000 μg to about 60000 μg, about 40000 μg to about 50000 μg, about 50000 μg to about 100000 μg, about 50000 μg to about 70000 μg, about 60000 μg to about 100000 μg or about 70000 μg to about 100000 μg.

In some embodiments, the dose of ETBR antagonist (e.g., specifically deuterated ETBR antagonist) as described herein is about 1.0 μg to about 10000 μg. In some embodiments, the dose is about 1.0 μg to about 5000 μg. In some embodiments, the dose is about 1.0 μg to about 2500 μg. In some embodiments, the dose is about 1.0 μg to about 1000 μg. In some embodiments, the dose is about 1.0 μg to about 500 μg. In some embodiments, the dose is about 1.0 μg to about 250 μg. In some embodiments, the dose is about 1.0 μg to about 100 μg. In some embodiments, the dose is about 1.0 μg to about 75 μg. In some embodiments, the dose is about 1.0 μg to about 50 μg. In some embodiments, the dose is about 1.0 μg to about 25 μg. In some embodiments, the dose is about 1.0 μg to about 10 μg. In some embodiments, the dose is about 10 μg to about 10000 μg. In some embodiments, the dose is about 10 μg to about 5000 μg. In some embodiments, the dose is about 10 μg to about 2500 μg. In some embodiments, the dose is about 10 μg to about 1000 μg. In some embodiments, the dose is about 10 μg to about 500 μg. In some embodiments, the dose is about 10 μg to about 250 μg. In some embodiments, the dose is about 10 μg to about 100 μg. In some embodiments, the dose is about 10 μg to about 75 μg. In some embodiments, the dose is about 10 μg to about 50 μg. In some embodiments, the dose is about 50 μg to about 10000 μg. In some embodiments, the dose is about 50 μg to about 5000 μg. In some embodiments, the dose is about 50 μg to about 2500 μg. In some embodiments, the dose is about 50 μg to about 2000 μg. In some embodiments, the dose is about 50 μg to about 1000 μg. In some embodiments, the dose is about 50 μg to about 750 μg. In some embodiments, the dose is about 50 μg to about 500 μg. In some embodiments, the dose is about 50 μg to about 250 μg. In some embodiments, the dose is about 50 μg to about 200 μg. In some embodiments, the dose is about 50 μg to about 100 μg. In some embodiments, the dose is about 100 μg to about 10000 μg. In some embodiments, the dose is about 100 μg to about 5000 μg. In some embodiments, the dose is about 100 μg to about 2500 μg. In some embodiments, the dose is about 100 μg to about 2000 μg. In some embodiments, the dose is about 100 μg to about 1500 μg. In some embodiments, the dose is about 100 μg to about 1250 μg. In some embodiments, the dose is about 100 μg to about 1000 μg. In some embodiments, the dose is about 100 μg to about 750 μg. In some embodiments, the dose is about 100 μg to about 500 μg. In some embodiments, the dose is about 100 μg to about 250 μg. In some embodiments, the dose is about 100 μg to about 200 μg. In some embodiments, the dose is about 250 μg to about 10000 μg. In some embodiments, the dose is about 250 μg to about 5000 μg. In some embodiments, the dose is about 250 μg to about 2500 μg. In some embodiments, the dose is about 250 μg to about 2000 μg. In some embodiments, the dose is about 250 μg to about 1500 μg. In some embodiments, the dose is about 250 μg to about 1250 μg. In some embodiments, the dose is about 250 μg to about 1000 μg. In some embodiments, the dose is about 250 μg to about 750 μg. In some embodiments, the dose is about 250 μg to about 500 μg. In some embodiments, the dose is about 500 μg to about 10000 μg. In some embodiments, the dose is about 500 μg to about 7000 μg. In some embodiments, the dose is about 500 μg to about 5000 μg. In some embodiments, the dose is about 500 μg to about 3000 μg. In some embodiments, the dose is about 500 μg to about 2500 μg. In some embodiments, the dose is about 500 μg to about 2000 μg. In some embodiments, the dose is about 500 μg to about 1500 μg. In some embodiments, the dose is about 500 μg to about 1250 μg. In some embodiments, the dose is about 500 μg to about 1000 μg. In some embodiments, the dose is about 500 μg to about 750 μg. In some embodiments, the dose is about 750 μg to about 20000 μg. In some embodiments, the dose is about 750 μg to about 15000 μg. In some embodiments, the dose is about 750 μg to about 10000 μg. In some embodiments, the dose is about 750 μg to about 7000 μg. In some embodiments, the dose is about 750 μg to about 5000 μg. In some embodiments, the dose is about 750 μg to about 3000 μg. In some embodiments, the dose is about 750 μg to about 2500 μg. In some embodiments, the dose is about 750 μg to about 2000 μg. In some embodiments, the dose is about 750 μg to about 1500 μg. In some embodiments, the dose is about 750 μg to about 1250 μg. In some embodiments, the dose is about 750 μg to about 1000 μg. In some embodiments, the dose is about 1000 μg to about 25000 μg. In some embodiments, the dose is about 1000 μg to about 20000 μg. In some embodiments, the dose is about 1000 μg to about 15000 μg. In some embodiments, the dose is about 1000 μg to about 10000 μg. In some embodiments, the dose is about 1000 μg to about 7000 μg. In some embodiments, the dose is about 1000 μg to about 5000 μg. In some embodiments, the dose is about 1000 μg to about 3000 μg. In some embodiments, the dose is about 1000 μg to about 2500 μg. In some embodiments, the dose is about 1000 μg to about 2000 μg. In some embodiments, the dose is about 1000 μg to about 1500 μg. In some embodiments, the dose is about 1500 μg to about 25000 μg. In some embodiments, the dose is about 1500 μg to about 20000 μg. In some embodiments, the dose is about 1500 μg to about 15000 μg. In some embodiments, the dose is about 1500 μg to about 10000 μg. In some embodiments, the dose is about 1500 μg to about 7000 μg. In some embodiments, the dose is about 1500 μg to about 5000 μg. In some embodiments, the dose is about 1500 μg to about 3000 μg. In some embodiments, the dose is about 1500 μg to about 2500 μg. In some embodiments, the dose is about 1500 μg to about 2000 μg. In some embodiments, the dose is about 2000 μg to about 30000 μg. In some embodiments, the dose is about 2000 μg to about 25000 μg. In some embodiments, the dose is about 2000 μg to about 20000 μg. In some embodiments, the dose is about 2000 μg to about 15000 μg. In some embodiments, the dose is about 2000 μg to about 10000 μg. In some embodiments, the dose is about 2000 μg to about 7000 μg. In some embodiments, the dose is about 2000 μg to about 5000 μg. In some embodiments, the dose is about 2000 μg to about 4000 μg. In some embodiments, the dose is about 2000 μg to about 3000 μg. In some embodiments, the dose is about 2000 μg to about 2500 μg. In some embodiments, the dose is about 2500 μg to about 30000 μg. In some embodiments, the dose is about 2500 μg to about 25000 μg. In some embodiments, the dose is about 2500 μg to about 20000 μg. In some embodiments, the dose is about 2500 μg to about 15000 μg. In some embodiments, the dose is about 2500 μg to about 12000 μg. In some embodiments, the dose is about 2500 μg to about 10000 μg. In some embodiments, the dose is about 2500 μg to about 7000 μg. In some embodiments, the dose is about 2500 μg to about 5000 μg. In some embodiments, the dose is about 2500 μg to about 4000 μg. In some embodiments, the dose is about 2500 μg to about 3000 μg. In some embodiments, the dose is about 3000 μg to about 50000 μg. In some embodiments, the dose is about 3000 μg to about 25000 μg. In some embodiments, the dose is about 3000 μg to about 20000 μg. In some embodiments, the dose is about 3000 μg to about 15000 μg. In some embodiments, the dose is about 3000 μg to about 12000 μg. In some embodiments, the dose is about 3000 μg to about 10000 μg. In some embodiments, the dose is about 3000 μg to about 7000 μg. In some embodiments, the dose is about 3000 μg to about 6000 μg. In some embodiments, the dose is about 3000 μg to about 5000 μg. In some embodiments, the dose is about 3000 μg to about 4000 μg. In some embodiments, the dose is about 4000 μg to about 50000 μg. In some embodiments, the dose is about 4000 μg to about 30000 μg. In some embodiments, the dose is about 4000 μg to about 25000 μg. In some embodiments, the dose is about 4000 μg to about 20000 μg. In some embodiments, the dose is about 4000 μg to about 15000 μg. In some embodiments, the dose is about 4000 μg to about 12000 μg. In some embodiments, the dose is about 4000 μg to about 10000 μg. In some embodiments, the dose is about 4000 μg to about 8000 μg. In some embodiments, the dose is about 4000 μg to about 6000 μg. In some embodiments, the dose is about 4000 μg to about 5000 μg. In some embodiments, the dose is about 5000 μg to about 50000 μg. In some embodiments, the dose is about 5000 μg to about 40000 μg. In some embodiments, the dose is about 5000 μg to about 30000 μg. In some embodiments, the dose is about 5000 μg to about 25000 μg. In some embodiments, the dose is about 5000 μg to about 20000 μg. In some embodiments, the dose is about 5000 μg to about 15000 μg. In some embodiments, the dose is about 5000 μg to about 12000 μg. In some embodiments, the dose is about 5000 μg to about 10000 μg. In some embodiments, the dose is about 5000 μg to about 8000 μg. In some embodiments, the dose is about 5000 μg to about 7000 μg. In some embodiments, the dose is about 5000 μg to about 6000 μg. In some embodiments, the dose is about 6000 μg to about 50000 μg. In some embodiments, the dose is about 6000 μg to about 30000 μg. In some embodiments, the dose is about 6000 μg to about 25000 μg. In some embodiments, the dose is about 6000 μg to about 20000 μg. In some embodiments, the dose is about 6000 μg to about 17000 μg. In some embodiments, the dose is about 6000 μg to about 15000 μg. In some embodiments, the dose is about 6000 μg to about 12000 μg. In some embodiments, the dose is about 6000 μg to about 10000 μg. In some embodiments, the dose is about 6000 μg to about 9000 μg. In some embodiments, the dose is about 6000 μg to about 8000 μg. In some embodiments, the dose is about 6000 μg to about 7000 μg. In some embodiments, the dose is about 7000 μg to about 70000 μg. In some embodiments, the dose is about 7000 μg to about 50000 μg. In some embodiments, the dose is about 7000 μg to about 40000 μg. In some embodiments, the dose is about 7000 μg to about 30000 μg. In some embodiments, the dose is about 7000 μg to about 25000 μg. In some embodiments, the dose is about 7000 μg to about 20000 μg. In some embodiments, the dose is about 7000 μg to about 17000 μg. In some embodiments, the dose is about 7000 μg to about 15000 μg. In some embodiments, the dose is about 7000 μg to about 12000 μg. In some embodiments, the dose is about 7000 μg to about 10000 μg. In some embodiments, the dose is about 7000 μg to about 8000 μg. In some embodiments, the dose is about 8000 μg to about 80000 μg. In some embodiments, the dose is about 8000 μg to about 60000 μg. In some embodiments, the dose is about 8000 μg to about 50000 μg. In some embodiments, the dose is about 8000 μg to about 30000 μg. In some embodiments, the dose is about 8000 μg to about 25000 μg. In some embodiments, the dose is about 8000 μg to about 20000 μg. In some embodiments, the dose is about 8000 μg to about 17000 μg. In some embodiments, the dose is about 8000 μg to about 15000 μg. In some embodiments, the dose is about 8000 μg to about 12000 μg. In some embodiments, the dose is about 8000 μg to about 10000 μg. In some embodiments, the dose is about 10000 μg to about 100000 μg. In some embodiments, the dose is about 10000 μg to about 80000 μg. In some embodiments, the dose is about 10000 μg to about 70000 μg. In some embodiments, the dose is about 10000 μg to about 60000 μg. In some embodiments, the dose is about 10000 μg to about 50000 μg. In some embodiments, the dose is about 10000 μg to about 40000 μg. In some embodiments, the dose is about 10000 μg to about 30000 μg. In some embodiments, the dose is about 10000 μg to about 25000 μg. In some embodiments, the dose is about 10000 μg to about 20000 μg. In some embodiments, the dose is about 10000 μg to about 18000 μg. In some embodiments, the dose is about 10000 μg to about 16000 μg. In some embodiments, the dose is about 10000 μg to about 15000 μg. In some embodiments, the dose is about 10000 μg to about 12000 μg. In some embodiments, the dose is about 12000 μg to about 100000 μg. In some embodiments, the dose is about 12000 μg to about 90000 μg. In some embodiments, the dose is about 12000 μg to about 80000 μg. In some embodiments, the dose is about 12000 μg to about 70000 μg. In some embodiments, the dose is about 12000 μg to about 60000 μg. In some embodiments, the dose is about 12000 μg to about 50000 μg. In some embodiments, the dose is about 12000 μg to about 40000 μg. In some embodiments, the dose is about 12000 μg to about 30000 μg. In some embodiments, the dose is about 12000 μg to about 25000 μg. In some embodiments, the dose is about 12000 μg to about 20000 μg. In some embodiments, the dose is about 12000 μg to about 18000 μg. In some embodiments, the dose is about 12000 μg to about 16000 μg. In some embodiments, the dose is about 12000 μg to about 15000 μg. In some embodiments, the dose is about 12000 μg to about 14000 μg. In some embodiments, the dose is about 15000 μg to about 100000 μg. In some embodiments, the dose is about 15000 μg to about 90000 μg. In some embodiments, the dose is about 15000 μg to about 80000 μg. In some embodiments, the dose is about 15000 μg to about 70000 μg. In some embodiments, the dose is about 15000 μg to about 60000 μg. In some embodiments, the dose is about 15000 μg to about 50000 μg. In some embodiments, the dose is about 15000 μg to about 40000 μg. In some embodiments, the dose is about 15000 μg to about 30000 μg. In some embodiments, the dose is about 15000 μg to about 25000 μg. In some embodiments, the dose is about 15000 μg to about 20000 μg. In some embodiments, the dose is about 20000 μg to about 100000 μg. In some embodiments, the dose is about 20000 μg to about 90000 μg. In some embodiments, the dose is about 20000 μg to about 80000 μg. In some embodiments, the dose is about 20000 μg to about 70000 μg. In some embodiments, the dose is about 20000 μg to about 60000 μg. In some embodiments, the dose is about 20000 μg to about 50000 μg. In some embodiments, the dose is about 20000 μg to about 40000 μg. In some embodiments, the dose is about 20000 μg to about 30000 μg. In some embodiments, the dose is about 20000 μg to about 25000 μg. In some embodiments, the dose is about 25000 μg to about 100000 μg. In some embodiments, the dose is about 25000 μg to about 90000 μg. In some embodiments, the dose is about 25000 μg to about 80000 μg. In some embodiments, the dose is about 25000 μg to about 70000 μg. In some embodiments, the dose is about 25000 μg to about 60000 μg. In some embodiments, the dose is about 25000 μg to about 50000 μg. In some embodiments, the dose is about 25000 μg to about 40000 μg. In some embodiments, the dose is about 25000 μg to about 30000 μg. In some embodiments, the dose is about 30000 μg to about 100000 μg. In some embodiments, the dose is about 30000 μg to about 90000 μg. In some embodiments, the dose is about 30000 μg to about 80000 μg. In some embodiments, the dose is about 30000 μg to about 70000 μg. In some embodiments, the dose is about 30000 μg to about 60000 μg. In some embodiments, the dose is about 30000 μg to about 50000 μg. In some embodiments, the dose is about 30000 μg to about 40000 μg. In some embodiments, the dose is about 40000 μg to about 100000 μg. In some embodiments, the dose is about 40000 μg to about 80000 μg. In some embodiments, the dose is about 40000 μg to about 60000 μg. In some embodiments, the dose is about 40000 μg to about 50000 μg. In some embodiments, the dose is about 50000 μg to about 100000 μg. In some embodiments, the dose is about 50000 μg to about 70000 μg. In some embodiments, the dose is about 60000 μg to about 100000 μg or about 70000 μg to about 100000 μg.

In some embodiments, the dose is about 5 μg, about 10 μg, about 25 μg, about 50 μg, about 75 μg, about 100 μg, about 125 μg, about 150 μg, about 175 μg, about 200 μg, about 225 μg, about 250 μg, about 275 μg, about 300 μg, about 325 μg, about 350 μg, about 375 μg, about 400 μg, about 425 μg, about 450 μg, about 475 μg, about 500 μg, about 550 μg, about 600 μg, about 650 μg, about 700 μg, about 750 μg, about 800 μg, about 850 μg, about 1000 μg, about 1250 μg, about 1500 μg, about 1750 μg, about 2000 μg, about 2500 μg, about 3000 μg, about 3500 μg, about 4000 μg, about 4500 μg, about 5000 μg, about 6000 μg, about 7000 μg, about 8000 μg, about 9000 μg, about 10000 μg, about 11000 μg, about 12000 μg, about 13000 μg, about 14000 μg, about 15000 μg, about 16000 μg, about 17000 μg, about 18000 μg, about 19000 μg, about 20000 μg, about 21000 μg, about 22000 μg, about 23000 μg, about 24000 μg, about 25000 μg, about 26000 μg, about 27000 μg, about 28000 μg, about 29000 μg, about 30000 μg, about 40000 μg, about 50000 μg, about 60000 μg, about 70000 μg, about 80000 μg, about 90000 μg or about 100000 μg.

In some embodiments, the dose is about 100 μg, about 150 μg, about 300 μg, about 500 μg, about 750 μg, about 1000 μg, about 1500 μg, about 2500 μg, about 300 μg, about 3000 μg, about 3500 μg, about 4000 μg, about 4500 μg, about 5000 μg, about 6000 μg, about 7000 μg, about 8000 μg, about 9000 μg, about 10000 μg, about 11000 μg, about 12000 μg, about 13000 μg, about 14000 μg, about 15000 μg, about 16000 μg, about 17000 μg, about 18000 μg, about 19000 μg, about 20000 μg, about 21000 μg, about 22000 μg, about 23000 μg, about 24000 μg, about 25000 μg, about 26000 μg, about 27000 μg, about 28000 μg, about 29000 μg, about 30000 μg, about 40000 μg or about 50000 μg.

In some embodiments, the dose is about 300 μg, about 300 μg, about 750 μg, about 1000 μg, about 1500 μg, about 2000 μg, about 2500, about 3000 μg, about 4000 μg or about 5000 μg. In some embodiments, the dose is about 100 μg, about 150 μg, about 300 μg, about 750 μg, about 1000 μg, about 1500 μg, about 2000 μg, about 3000 μg, about 3500 μg, about 4000 μg, about 4500 μg, about 5000 μg, about 6000 μg, about 7000 μg, about 8000 μg, about 9000 μg, about 10000 μg, about 11000 μg, about 12000 μg, about 13000 μg, about 14000 μg, about 15000 μg, about 16000 μg, about 17000 μg, about 18000 μg, about 19000 μg, about 20000 μg, about 21000 μg, about 22000 μg, about 23000 μg, about 24000 μg or about 25000 μg.

In some embodiments, the dose is about 100 μg, about 150 μg, about 300 μg, about 750 μg, about 1000 μg, about 1500 μg, about 2000 μg, about 3000 μg, about 3500 μg, about 4000 μg, about 4500 μg, about 5000 μg, about 6000 μg, about 7000 μg, about 8000 μg, about 9000 μg, about 10000 μg, about 11000 μg, about 12000 μg, about 13000 μg, about 14000 μg, about 15000 μg, about 16000 μg, about 17000 μg, about 18000 μg, about 19000 μg, about 20000 μg, about 21000 μg, about 22000 μg, about 23000 μg, about 24000 μg, about 25000 μg, about 26000 μg, about 27000 μg, about 28000 μg, about 29000 μg, about 30000 μg, about 40000 μg or about 50000 μg.

In some embodiments, the dose is about 2000 μg, about 2500 μg, about 3000 μg, about 3500 μg, about 4000 μg, about 4500 μg, about 5000 μg, about 6000 μg, about 7000 μg, about 8000 μg, about 9000 μg, about 10000 μg, about 11000 μg, about 12000 μg, about 13000 μg, about 14000 μg, about 15000 μg, about 16000 μg, about 17000 μg, about 18000 μg, about 19000 μg, about 20000 μg, about 21000 μg, about 22000 μg, about 23000 μg, about 24000 μg or about 25000 μg.

In some embodiments, the dose is about 2000 μg, about 2500 μg, about 3000 μg, about 3500 μg, about 4000 μg, about 4500 μg, about 5000 μg, about 6000 μg, about 7000 μg, about 8000 μg, about 9000 μg or about 10000 μg.

In some embodiments, the dose is about 5000 μg, about 6000 μg, about 7000 μg, about 8000 μg, about 9000 μg or about 10000 μg.

In some embodiments, the dose is about 7000 μg, about 8000 μg, about 9000 μg or about 10000 μg, about 11000 μg, about 12000 μg, about 13000 μg, about 14000 μg, about 15000 μg, about 16000 μg, about 17000 μg, about 18000 μg, about 19000 μg, about 20000 μg, about 21000 μg, about 22000 μg, about 23000 μg, about 24000 μg, about 25000 μg, about 26000 μg, about 27000 μg, about 28000 μg, about 29000 μg, about 30000 μg, about 40000 μg or about 50000 μg.

In some embodiments, the dose is about 100 μg, about 150 μg, about 300 μg, about 750 μg, about 1000 μg, about 1500 μg, about 2000 μg, about 25000 μg, about 3000 μg, about 4000 μg or about 5000 μg.

In some embodiments, the dose is about 10 μg, about 50 μg, about 100 μg, about 150 μg, about 300 μg, about 750 μg or about 1000 μg. In some embodiments, the dose is about 50 μg, about 100 μg, about 150 μg, about 300 μg, about 750 μg or about 1000 μg. In some embodiments, the dose is about 100 μg, about 150 μg, about 300 μg, about 750 μg, about 1000 μg, about 1500 μg, about 2000 μg, about 3000 μg, about 4000 μg or about 5000 μg. In some embodiments, the dose is about 100 μg, about 150 μg, about 300 μg, about 750 μg, about 1000 μg, about 1500 μg, about 2000 μg or about 3000 μg. In some embodiments, the dose is about 300 μg, about 750 μg, about 1000 μg, about 1500 μg or about 2000 μg. In some embodiments, the dose is about 150 μg, about 300 μg, about 750 μg or about 1000 μg. In some embodiments, the dose is about 300 μg, about 750 μg or about 1000 μg. In some embodiments, the dose is about 150 μg, about 300 μg or about 750 μg. In some embodiments, the dose is about 300 μg, about 750 μg or about 1000 μg. In some embodiments, the dose is about 150 μg or about 300 μg. In some embodiments, the dose is about 300 μg or about 750 μg. In some embodiments, the dose is about 750 μg or about 1000 μg.

In some embodiments, the dose is about 5 μg. In some embodiments, the dose is about 10 μg. In some embodiments, the dose is about 25 μg. In some embodiments, the dose is about 50 μg. In some embodiments, the dose is about 75 μg. In some embodiments, the dose is about 100 μg. In some embodiments, the dose is about 125 μg. In some embodiments, the dose is about 150 μg. In some embodiments, the dose is about 175 μg. In some embodiments, the dose is about 200 μg. In some embodiments, the dose is about 225 μg. In some embodiments, the dose is about 250 μg. In some embodiments, the dose is about 275 μg. In some embodiments, the dose is about 300 μg. In some embodiments, the dose is about 325 μg. In some embodiments, the dose is about 350 μg. In some embodiments, the dose is about 375 μg. In some embodiments, the dose is about 400 μg. In some embodiments, the dose is about 425 μg. In some embodiments, the dose is about 450 μg. In some embodiments, the dose is about 475 μg. In some embodiments, the dose is about 500 μg. In some embodiments, the dose is about 550 μg. In some embodiments, the dose is about 600 μg. In some embodiments, the dose is about 650 μg. In some embodiments, the dose is about 700 μg. In some embodiments, the dose is about 750 μg. In some embodiments, the dose is about 800 μg. In some embodiments, the dose is about 850 μg. In some embodiments, the dose is about 1000 μg. In some embodiments, the dose is about 1250 μg. In some embodiments, the dose is about 1500 μg. In some embodiments, the dose is about 1750 μg. In some embodiments, the dose is about 2000 μg. In some embodiments, the dose is about 2500 μg. In some embodiments, the dose is about 3000 μg. In some embodiments, the dose is about 3500 μg. In some embodiments, the dose is about 4000 μg. In some embodiments, the dose is about 4500 μg. In some embodiments, the dose is about 5000 μg. In some embodiments, the dose is about 6000 μg. In some embodiments, the dose is about 7000 μg. In some embodiments, the dose is about 8000 μg. In some embodiments, the dose is about 9000 μg. In some embodiments, the dose is about 10000 μg. In some embodiments, the dose is about 11000 μg. In some embodiments, the dose is about 12000 μg. In some embodiments, the dose is about 13000 μg. In some embodiments, the dose is about 14000 μg. In some embodiments, the dose is about 15000 μg. In some embodiments, the dose is about 16000 μg. In some embodiments, the dose is about 17000 μg. In some embodiments, the dose is about 18000 μg. In some embodiments, the dose is about 19000 μg. In some embodiments, the dose is about 20000 μg. In some embodiments, the dose is about 21000 μg. In some embodiments, the dose is about 22000 μg. In some embodiments, the dose is about 23000 μg. In some embodiments, the dose is about 24000 μg. In some embodiments, the dose is about 25000 μg. In some embodiments, the dose is about 26000 μg. In some embodiments, the dose is about 27000 μg. In some embodiments, the dose is about 28000 μg. In some embodiments, the dose is about 29000 μg. In some embodiments, the dose is about 30000 μg. In some embodiments, the dose is about 40000 μg. In some embodiments, the dose is about 50000 μg. In some embodiments, the dose is about 60000 μg. In some embodiments, the dose is about 70000 μg. In some embodiments, the dose is about 80000 μg. In some embodiments, the dose is about 90000 μg. In some embodiments, the dose is about 100000 μg.

In some embodiments, the ETBR antagonist (e.g., the specifically deuterated ETBR antagonist) is administered daily. In some embodiments, the ETBR antagonist (e.g., the specifically deuterated ETBR antagonist) is administered every other day. In some embodiments, the ETBR antagonist (e.g., the specifically deuterated ETBR antagonist) is administered every four days. In some embodiments, the ETBR antagonist (e.g., the specifically deuterated ETBR antagonist) is administered every three days. In some embodiments, the ETBR antagonist (e.g., the specifically deuterated ETBR antagonist) is administered every five days. In some embodiments, the ETBR antagonist (e.g., the specifically deuterated ETBR antagonist) is administered every six days. In some embodiments, the ETBR antagonist (e.g., the specifically deuterated ETBR antagonist) is administered weekly. In some embodiments, the ETBR antagonist (e.g., the specifically deuterated ETBR antagonist) is administered every ten days. In some embodiments, the ETBR antagonist (e.g., the specifically deuterated ETBR antagonist) is administered every two weeks. In some embodiments, the ETBR antagonist (e.g., the specifically deuterated ETBR antagonist) is administered every three weeks.

### Routes of Administration

Disclosed herein is a variety of routes of administration for the pharmaceutical compositions disclosed herein. The compounds as described herein may, in accordance with the disclosure, be administered in single or divided doses by the oral, parenteral or topical routes. Administration of the active compound may range from continuous (intravenous drip) to several oral administrations per day (for example, Q.O.D. or Q.I.D.) and may include oral, topical, parenteral, intramuscular, intravenous, sub-cutaneous, transdermal (which may include a penetration enhancement agent), buccal, sublingual and suppository administration, among other routes of administration. Enteric coated oral tablets may also be used to enhance bioavailability of the compounds from an oral route of administration. The most effective dosage form will depend upon the pharmacokinetics of the particular agent(s) chosen as well as the severity of disease in the patient. Administration of compounds according to the present disclosure as sprays, mists, or aerosols for intra-nasal, intra-tracheal or pulmonary administration may also be used. The present disclosure therefore also is directed to pharmaceutical compositions comprising an effective amount of compound as described herein, optionally in combination with a pharmaceutically acceptable carrier, additive or excipient. Compounds according to the present disclosure may be administered in immediate release, intermediate release or sustained or controlled release forms. In some embodiments, sustained or controlled release forms are y administered orally, but also in suppository and transdermal or other topical forms. Intramuscular injections in liposomal form may also be used to control or sustain the release of compound at an injection site.

In some embodiments, the pharmaceutical compositions as described herein is administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intraperitoneally or intravenously.

In some embodiments, sterile injectable forms of the compositions as described herein are aqueous or oleaginous suspension. These suspensions may be formulated using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives that are useful in the preparation of injectables, are natural pharmaceutically-acceptable oils, such as olive oil, castor oil or soybean oil, especially in their polyoxyethylated versions. These oil solutions or suspensions may also contain a long-chain alcohol diluent or dispersant, such as Ph. Helv or similar alcohol.

In some embodiments, the pharmaceutical compositions as described herein are orally administered in any orally acceptable dosage form including, but not limited to, capsules, tablets, aqueous suspensions or solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are used orally, the active ingredient is combined with emulsifying and suspending agents. If desired, certain sweetening, flavoring or coloring agents may also be added.

In some embodiments, the pharmaceutical compositions as described herein are administered in the form of suppositories for rectal administration. These can be prepared by mixing the agent with a suitable non-irritating excipient, which is solid at room temperature but liquid at rectal temperature and therefore will melt in the rectum to release the drug. Such materials include cocoa butter, beeswax and polyethylene glycols.

In some embodiments, the pharmaceutical compositions as described herein are administered topically. Suitable topical formulations are readily prepared for each of these areas or organs. Topical application for the lower intestinal tract can be effected in a rectal suppository formulation (see above) or in a suitable enema formulation. Topically-acceptable transdermal patches may also be used.

In some embodiments, for topical applications, the pharmaceutical compositions are formulated in a suitable ointment containing the active component suspended or dissolved in one or more carriers. Carriers for topical administration of the compounds of this disclosure include, but are not limited to, mineral oil, liquid petrolatum, DMSO, white petrolatum, propylene glycol, polyoxyethylene, polyoxypropylene compound, emulsifying wax and water. In some embodiments, the compounds may be coated onto a stent which is to be surgically implanted into a patient in order to inhibit or reduce the likelihood of occlusion occurring in the stent in the patient.

In some embodiments, the pharmaceutical compositions are formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

In some embodiments, for ophthalmic use, the pharmaceutical compositions are formulated as micronized suspensions in isotonic, pH adjusted sterile saline, or as solutions in isotonic, pH adjusted sterile saline, either with or without a preservative such as benzalkonium chloride. Alternatively, for ophthalmic uses, the pharmaceutical compositions may be formulated in an ointment such as petrolatum.

In some embodiments, the pharmaceutical compositions as described herein are administered by nasal aerosol or inhalation. Such compositions are prepared according to techniques described herein relating to pharmaceutical compositions and may be prepared as solutions in saline, employing benzyl alcohol or other suitable preservatives, absorption promoters to enhance bioavailability, fluorocarbons, and/or other conventional solubilizing or dispersing agents. In some embodiments, the description provides formulations comprising liposomes including an effective amount (e.g., a synergistically effective amount) of an a ETBR antagonist.

In some embodiments, the compositions should be formulated to contain between about 0.05 milligram to about 750 milligrams or more, for example about 1 milligram to about 600 milligrams, or about 10 milligrams to about 500 milligrams of active ingredient, alone or in combination with another compound according to the present disclosure. It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease or condition being treated.

In some embodiments, a patient or subject in need of therapy using compounds according to the methods described herein is treated by administering to the patient (subject) an effective amount of the compound according to the present disclosure including pharmaceutically acceptable salts, solvates or polymorphs thereof optionally in a pharmaceutically acceptable carrier or diluent, either alone, or in combination with other known erythropoiesis stimulating agents as otherwise identified herein.

In some embodiments, the compounds or compositions herein are administered orally, parenterally, intradermally, by an injection (intravenously, subcutaneously, or intramuscularly), topically, including transdermally, in liquid, cream, gel, or solid form, or by aerosol form.

In some embodiments, the active ingredients are included in the pharmaceutically acceptable carrier or diluent in an amount sufficient to deliver to a patient a therapeutically effective amount for the desired indication, without causing serious toxic effects in the patient treated. An exemplary dose of the active compound for all of the herein-mentioned conditions is in the range from about 10 ng/kg to 300 ng/kg, about 10 ng/kg to 1 μg/kg, about 1 μg/kg to 10 μg/kg, about 10 μg/kg to 100 μg/kg, about 100 μg/kg to 1000 μg/kg, about 1 mg/kg to 30 mg/kg, about 1 mg/kg to 300 mg/kg, or 0.1 to 100 mg/kg per day, more generally 0.5 to about 25 mg per kilogram body weight of the recipient/patient per day. A typical topical dosage will range from 0.01-5% wt/wt in a suitable carrier.

In some embodiments, the active ingredient herein is conveniently administered in any suitable unit dosage form, including but not limited to, one containing less than 1 μg, 1 μg to 5000 μg, for example 50 to 3000 μg of active ingredient per unit dosage form.

In some embodiments, the active ingredient is administered to achieve peak plasma concentrations of the active compound of about 0.00001-30 mM, for example about 0.1-30 μM. This may be achieved, for example, by the intravenous injection of a solution or formulation of the active ingredient, optionally in saline, or an aqueous medium or administered as a bolus of the active ingredient. Oral administration is also appropriate to generate effective plasma concentrations of active agent.

### Chimeric Antigen Receptor-Expressing Immune Cells

Combination therapies and methods described herein generally comprise a chimeric antigen-receptor (CAR) expressing immune cell. A “chimeric antigen receptor” (CAR) refers to a synthetic receptor that mimics immune receptor activation to direct immune cell activity against a target of interest. CARs consist essentially of (a) an extracellular domain that binds a target of interest (e.g., a single-chain variable fragment (scFv)), (b) a transmembrane domain, and (c) an intracellular domain that activates immune signaling (e.g., a CD3z domain). CARs can be expressed in multiple different types of immune cells useful for methods described herein. In some embodiments, the CAR is expressed in T cells (e.g., CD4 T cells, CD8 T cells, T_{C}1 cells, T_{C}2 cells, γδ T cells, , T_{H}1 cells, T_{H}2 cells, T_{H}3, T_{H}17 cells, T_{FH} cells, T_{reg} cells, memory T cells, NK T cells) or NK cells. In some embodiments, the CAR is expressed in T cells (“CAR-T cells”). In some embodiments, the CAR is expressed in NK cells (“CAR-NK cells”).

In some embodiments, CARs useful in a method described herein specifically bind a tumor-associated antigen. “Tumor-associated antigens” are proteins that are produced by tumor cells that elicit an immune response. Tumor-associated antigens include, for example, human epidermal growth factor receptor 2 (HER2) In some embodiments, the tumor-associated antigen is (human epidermal growth factor receptor 2) HER2.

In some embodiments, the dosage of CAR-expressing immune cells is 10⁴ to 10⁹ cells/kg body weight, 10⁵ to 10⁹ cells/kg body weight, 10⁶ to 10⁹ cells/kg body weight, 10⁷ to 10⁹ cells/kg body weight, 10⁸ to 10⁹ cells/kg body weight, 10⁴ to 10⁸ cells/kg body weight, 10⁵ to 10⁸ cells/kg body weight, 10⁶ to 10⁸ cells/kg body weight, 10⁷ to 10⁸ cells/kg body weight, 10⁴ to 10⁷ cells/kg body weight, 10⁵ to 10⁷ cells/kg body weight, 10⁶ to 10⁷ cells/kg body weight, 10⁴ to 10⁶ cells/kg body weight, 10⁵ to 10⁶ cells/kg body weight, or 10⁴ to 10⁵ cells/kg body weight.

In some embodiments, the CAR-expressing immune cells are administered as a single infusion. In some embodiments, the CAR-expressing immune cells are administered as multiple infusions (e.g., 2-20 infusions). In some embodiments, the CAR-expressing immune cells are administered as two infusions. In some embodiments, the CAR-expressing immune cells are administered as three infusions. In some embodiments, the CAR-expressing immune cells are administered as four infusions. In some embodiments, the CAR-expressing immune cells are administered as five infusions. In some embodiments, the CAR-expressing immune cells are administered as six infusions. In some embodiments, the CAR-expressing immune cells are administered as seven infusions. In some embodiments, the CAR-expressing immune cells are administered as eight infusions. In some embodiments, the CAR-expressing immune cells are administered as nine infusions. In some embodiments, the CAR-expressing immune cells are administered as ten infusions. In some embodiments, the CAR-expressing immune cells are administered as eleven infusions. In some embodiments, the CAR-expressing immune cells are administered as twelve infusions. In some embodiments, the CAR-expressing immune cells are administered as thirteen infusions. In some embodiments, the CAR-expressing immune cells are administered as fourteen infusions. In some embodiments, the CAR-expressing immune cells are administered as sixteen infusions. In some embodiments, the CAR-expressing immune cells are administered as seventeen infusions. In some embodiments, the CAR-expressing immune cells are administered as eighteen infusions. In some embodiments, the CAR-expressing immune cells are administered as nineteen infusions. In some embodiments, the CAR-expressing immune cells are administered as twenty infusions.

In some embodiments, the CAR-expressing immune cells are administered at weekly intervals. In some embodiments, the CAR-expressing immune cells are administered at two-weeks intervals. In some embodiments, the CAR-expressing immune cells are administered at three-weeks intervals. In some embodiments, the CAR-expressing immune cells are administered at four-weeks intervals. In some embodiments, the CAR-expressing immune cells are administered at five-weeks intervals. In some embodiments, the CAR-expressing immune cells are administered at six-weeks intervals. In some embodiments, the CAR-expressing immune cells are administered at seven-weeks intervals. In some embodiments, the CAR-expressing immune cells are administered at eight-weeks intervals. In some embodiments, the CAR-expressing immune cells are administered at nine-weeks intervals. In some embodiments, the CAR-expressing immune cells are administered at ten-weeks intervals. In some embodiments, the CAR-expressing immune cells are administered at eleven-weeks intervals. In some embodiments, the CAR-expressing immune cells are administered at twelve-weeks intervals.

In some embodiments, the CAR-expressing immune cells are administered at monthly intervals. In some embodiments, the CAR-expressing immune cells are administered at two-months intervals. In some embodiments, the CAR-expressing immune cells are administered at three-months intervals. In some embodiments, the CAR-expressing immune cells are administered at four-months intervals. In some embodiments, the CAR-expressing immune cells are administered at five-months intervals. In some embodiments, the CAR-expressing immune cells are administered at six-months intervals. In some embodiments, the CAR-expressing immune cells are administered at seven-months intervals. In some embodiments, the CAR-expressing immune cells are administered at eight-months intervals. In some embodiments, the CAR-expressing immune cells are administered at nine-months intervals. In some embodiments, the CAR-expressing immune cells are administered at ten-months intervals. In some embodiments, the CAR-expressing immune cells are administered at eleven-months intervals. In some embodiments, the CAR-expressing immune cells are administered at twelve-months intervals.

### Immune Checkpoint Inhibitors

In various embodiments, methods disclosed herein comprise administering to a subject a combination therapy including an immune checkpoint inhibitor (ICPI). In some embodiments, the ICPI is an anti-PD1 antibody, an anti-PD-L1 antibody, an anti-CTLA-4 antibody, an anti-LAG-3 antibody, or a CISH inhibitor. In some embodiments, the ICPI is an anti-PD1 antibody. In some embodiments, the anti-PD1 antibody is nivolumab, pembrolizumab, pidilizumab, or any combination thereof. In some embodiments, the anti-PD1 antibody is nivolumab. In some embodiments, the anti-PD1 antibody is pembrolizumab. In some embodiments, the anti-PD1 antibody is pidilizumab. In some embodiments, the ICPI is an anti-PD-L1 antibody. In some embodiments, the anti-PD-L1 antibody is atezolizumab, MDX-1105, avelumab, durvalumab, or any combination thereof. In some embodiments, the anti-PD-L1 antibody is atezolizumab. In some embodiments, the anti-PD-L1 antibody is MDX-1105. In some embodiments, the anti-PD-L1 antibody is avelumab. In some embodiments, the anti-PD-L1 antibody is durvalumab.

In some embodiments, a dosage of the ICPI is about 0.1 mg/kg to about 9.0 mg/kg. For example, the dosage of the anti-PD1 antibody is about 0.1 mg/kg to about 9.0 mg/kg, about 0.1 mg/kg to about 8.0 mg/kg, about 0.1 mg/kg to about 7.0 mg/kg, about 0.1 mg/kg to about 6.0 mg/kg, about 0.1 mg/kg to about 5.0 mg/kg, about 0.1 mg/kg to about 4.0 mg/kg, about 0.1 mg/kg to about 3.0 mg/kg, about 0.1 mg/kg to about 2.0 mg/kg, about 0.1 mg/kg to about 1.0 mg/kg, about 1.0 mg/kg to about 9.0 mg/kg, about 1.0 mg/kg to about 8.0 mg/kg, about 1.0 mg/kg to about 7.0 mg/kg, about 1.0 mg/kg to about 6.0 mg/kg, about 1.0 mg/kg to about 5.0 mg/kg, about 1.0 mg/kg to about 4.0 mg/kg, about 1.0 mg/kg to about 3.0 mg/kg, about 1.0 mg/kg to about 2.0 mg/kg, about 2.0 mg/kg to about 9.0 mg/kg, about 2.0 mg/kg to about 8.0 mg/kg, about 2.0 mg/kg to about 7.0 mg/kg, about 2.0 mg/kg to about 6.0 mg/kg, about 2.0 mg/kg to about 5.0 mg/kg, about 2.0 mg/kg to about 4.0 mg/kg, about 2.0 mg/kg to about 3.0 mg/kg, about 3.0 mg/kg to about 9.0 mg/kg, about 3.0 mg/kg to about 8.0 mg/kg, about 3.0 mg/kg to about 7.0 mg/kg, about 3.0 mg/kg to about 6.0 mg/kg, about 3.0 mg/kg to about 5.0 mg/kg, about 3.0 mg/kg to about 4.0 mg/kg, about 4.0 mg/kg to about 9.0 mg/kg, about 4.0 mg/kg to about 8.0 mg/kg, about 4.0 mg/kg to about 7.0 mg/kg, about 4.0 mg/kg to about 6.0 mg/kg, about 4.0 mg/kg to about 5.0 mg/kg, about 5.0 mg/kg to about 9.0 mg/kg, about 5.0 mg/kg to about 8.0 mg/kg, about 5.0 mg/kg to about 7.0 mg/kg, about 5.0 mg/kg to about 6.0 mg/kg, about 6.0 mg/kg to about 9.0 mg/kg, about 6.0 mg/kg to about 8.0 mg/kg, about 6.0 mg/kg to about 7.0 mg/kg, about 7.0 mg/kg to about 9.0 mg/kg, about 7.0 mg/kg to about 8.0 mg/kg, or about 8.0 mg/kg to about 9.0 mg/kg.

In some embodiments, a dosage of the ICPI is about 10 mg to about 5000 mg. In some embodiments, a dosage of the ICPI is about 10 mg to about 900 mg. For example, the dosage of the anti-PD1 antibody is about 10 mg to about 900 mg, about 10 mg to about 800 mg, about 10 mg to about 700 mg, about 10 mg to about 600 mg, about 10 mg to about 500 mg, about 10 mg to about 400 mg, about 10 mg to about 300 mg, about 10 mg to about 200 mg, about 10 mg to about 100 mg, about 100 mg to about 900 mg, about 100 mg to about 800 mg, about 100 mg to about 700 mg, about 100 mg to about 600 mg, about 100 mg to about 500 mg, about 100 mg to about 400 mg, about 100 mg to about 300 mg, about 100 mg to about 200 mg, about 200 mg to about 900 mg, about 200 mg to about 800 mg, about 200 mg to about 700 mg, about 200 mg to about 600 mg, about 200 mg to about 500 mg, about 200 mg to about 400 mg, about 200 mg to about 300 mg, about 300 mg to about 900 mg, about 300 mg to about 800 mg, about 300 mg to about 700 mg, about 300 mg to about 600 mg, about 300 mg to about 500 mg, about 300 mg to about 400 mg, about 400 mg to about 900 mg, about 400 mg to about 800 mg, about 400 mg to about 700 mg, about 400 mg to about 600 mg, about 400 mg to about 500 mg, about 500 mg to about 900 mg, about 500 mg to about 800 mg, about 500 mg to about 700 mg, about 500 mg to about 600 mg, about 600 mg to about 900 mg, about 600 mg to about 800 mg, about 600 mg to about 700 mg, about 700 mg to about 900 mg, about 700 mg to about 800 mg, or about 800 mg to about 900 mg.

In some embodiments, a dosage of the ICPI is about 10 mg, about 20 mg, about 30 mg, about 40 mg, about 50 mg, about 70 mg, about 75 mg, about 80 mg, about 90 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2500 mg, about 3000 mg, about 3500 mg, about 4000 mg, about 4500 mg or about 5000 mg. In some embodiments, a dosage of the ICPI is about 10 mg, about 20 mg, about 30 mg, about 40 mg, about 50 mg, about 70 mg, about 75 mg, about 80 mg, about 90 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg or about 900 mg.

In some embodiments, the dosage of the ICPI is about 10 mg. In some embodiments, the dosage of the ICPI is about 20 mg. In some embodiments, the dosage of the ICPI is about 30 mg. In some embodiments, the dosage of the ICPI is about 40 mg. In some embodiments, the dosage of the ICPI is about 50 mg. In some embodiments, the dosage of the ICPI is about 70 mg. In some embodiments, the dosage of the ICPI is about 75 mg. In some embodiments, the dosage of the ICPI is about 80 mg. In some embodiments, the dosage of the ICPI is about 90 mg. In some embodiments, the dosage of the ICPI is about 100 mg. In some embodiments, the dosage of the ICPI is about 150 mg. In some embodiments, the dosage of the ICPI is about 200 mg. In some embodiments, the dosage of the ICPI is about 250 mg. In some embodiments, the dosage of the ICPI is about 300 mg. In some embodiments, the dosage of the ICPI is about 350 mg. In some embodiments, the dosage of the ICPI is about 400 mg. In some embodiments, the dosage of the ICPI is about 450 mg. In some embodiments, the dosage of the ICPI is about 500 mg. In some embodiments, the dosage of the ICPI is about 550 mg. In some embodiments, the dosage of the ICPI is about 600 mg. In some embodiments, the dosage of the ICPI is about 650 mg. In some embodiments, the dosage of the ICPI is about 700 mg. In some embodiments, the dosage of the ICPI is about 750 mg. In some embodiments, the dosage of the ICPI is about 800 mg. In some embodiments, the dosage of the ICPI is about 850 mg. In some embodiments, the dosage of the ICPI is about 900 mg. In some embodiments, the dosage of the ICPI is about 950 mg. In some embodiments, the dosage of the ICPI is about 1000 mg. In some embodiments, the dosage of the ICPI is about 1100 mg. In some embodiments, the dosage of the ICPI is about 1200 mg. In some embodiments, the dosage of the ICPI is about 1300 mg. In some embodiments, the dosage of the ICPI is about 1400 mg. In some embodiments, the dosage of the ICPI is about 1500 mg. In some embodiments, the dosage of the ICPI is about 1600 mg. In some embodiments, the dosage of the ICPI is about 1700 mg. In some embodiments, the dosage of the ICPI is about 1800 mg. In some embodiments, the dosage of the ICPI is about 1900 mg. In some embodiments, the dosage of the ICPI is about 2000 mg. In some embodiments, the dosage of the ICPI is about 2500 mg. In some embodiments, the dosage of the ICPI is about 3000 mg. In some embodiments, the dosage of the ICPI is about 3500 mg. In some embodiments, the dosage of the ICPI is about 4000 mg. In some embodiments, the dosage of the ICPI is about 4500 mg. In some embodiments, the dosage of the ICPI is about 5000 mg.

In some embodiments, the ICPI is administered daily. In some embodiments, the ICPI is administered every other day. In some embodiments, the ICPI is administered every three days. In some embodiments, the ICPI is administered every four days. In some embodiments, the ICPI is administered every five days. In some embodiments, the ICPI is administered every six days. In some embodiments, the ICPI is administered every seven days. In some embodiments, the ICPI is administered every eight days. In some embodiments, the ICPI is administered every nine days. In some embodiments, the ICPI is administered every ten days. In some embodiments, the ICPI is administered every eleven days. In some embodiments, the ICPI is administered every twelve days. In some embodiments, the ICPI is administered every thirteen days. In some embodiments, the ICPI is administered every fourteen days. In some embodiments, the ICPI is administered every fifteen days. In some embodiments, the ICPI is administered every sixteen days. In some embodiments, the ICPI is administered every seventeen days. In some embodiments, the ICPI is administered every eighteen days. In some embodiments, the ICPI is administered every nineteen days. In some embodiments, the ICPI is administered every twenty days. In some embodiments, the ICPI is administered every twenty one days. In some embodiments, the ICPI is administered weekly. In some embodiments, the ICPI is administered every two weeks. In some embodiments, the ICPI is administered every three weeks. In some embodiments, the ICPI is administered every four weeks. In some embodiments, the ICPI is administered every five weeks. In some embodiments, the ICPI is administered every six weeks. In some embodiments, the ICPI is administered every seven weeks. In some embodiments, the ICPI is administered every eight weeks. In some embodiments, the ICPI is administered every nine weeks. In some embodiments, the ICPI is administered every ten weeks. In some embodiments, the ICPI is administered every eleven weeks. In some embodiments, the ICPI is administered every twelve weeks.

### Kits

Disclosed herein is a kit or pharmaceutical compositions for treatment of a solid tumor cancer in a subject, e.g., a human subject, comprising an ETBR antagonist in an amount effective for use in a combination therapy with a CAR-expressing immune cell, and a pharmaceutically acceptable carrier. In some embodiments, the kit or pharmaceutical composition further comprises an ICPI (e.g., an anti-PD1 antibody). In some embodiments, the ETBR antagonist is a specifically deuterated ETBR antagonist, e.g., deuterated BQ-788 as described herein. In some embodiments, the ETBR antagonist, e.g., deuterated BQ-788, is disposed in a single container with the CAR-expressing immune cell. In some embodiments, the ETBR antagonist, e.g., deuterated BQ-788, is disposed in a first container, and the CAR-expressing immune cell is disposed in a second container, wherein the ETBR antagonist and the CAR-expressing immune cell are to be administered approximately contemporaneously.

In some embodiments, the description provides a kit for treatment of a solid tumor cancer in a human subject, comprising an amount of a CAR-expressing immune cell, a synergistically effective amount of an ETBR antagonist (e.g., a specifically deuterated ETBR antagonist as described herein), and a pharmaceutically acceptable carrier or excipient. . In some embodiments, the kit or pharmaceutical composition further comprises an ICPI (e.g., an anti-PD1 antibody). In some embodiments, the ETBR antagonist is deuterated BQ-788. In some embodiments, the ETBR antagonist is BQ-788-B (ENB-003). In some embodiments, the ICPI is an anti-PD1 antibody or anti-PD-L1 antibody.

### Methods of Treatment

Disclosed herein are methods for treating or ameliorating a cancer in a subject or a patient, e.g., an animal such as a human, comprising administering to a subject in need thereof an effective amount, e.g., a therapeutically effective amount or a synergistically effective amount, of a combination therapy as described herein, wherein the combination therapy is effective for treating or ameliorating the disease or disorder or symptom thereof in the subject. In some embodiments the combination therapy comprises a CAR-expressing immune cell and an ETBR antagonist (e.g., a deuterated ETBR antagonist). In some embodiments the combination therapy comprises a CAR-expressing immune cell, an ICPI, and an ETBR antagonist (e.g., a deuterated ETBR antagonist). In some embodiments, the ETBR antagonist (e.g., the deuterated ETBR antagonist) is administered 2, 3, 4, or 5 times as frequently as the CAR-expressing immune cell or ICPI, for example the ETBR antagonist is administered 3 times during 1-3 weeks (e.g., during about 2-3 weeks or during about 21 days) while the CAR-expressing immune cell is administered 1 or 2 times during the 1-3 weeks (e.g., during about 2-3 weeks or during about 21 days).

In some embodiments, the disease or disorder is an target-expressing cancer. In some embodiments, the cancer is refractory or insensitive to immune-based therapy (e.g., therapy with a checkpoint inhibitor and/or with CAR-expressing immune therapies, e.g., CAR-T therapies) or both. In some embodiments the cancer is newly diagnosed. In some embodiments, the cancer is relapsed or refractory. In some embodiments, the cancer is relapsed. In some embodiments, the cancer is refractory.

In some embodiments, the cancer is an ETBR related cancer. In some embodiments, the cancer is a HER2-expressing cancer. In some embodiments, the cancer is a HER2+ cancer. In some embodiments, the cancer is a HER2-low cancer. In some embodiments, the cancer is a hematological malignancy. In some embodiments, the cancer is a solid tumor. In some embodiments, the cancer is selected from malignant melanoma, malignant squamous cell carcinoma, glioblastoma, pancreatic cancer, colon cancer, breast cancer, ovarian cancer, prostate cancer, bladder cancer and kidney cancer. In some embodiments, the cancer is selected from breast cancer, gastric cancer, esophageal cancer, ovarian cancer, endometrial cancer, bladder cancer, lung cancer, cervical cancer, colon cancer, and head and neck cancer. In some embodiments, the cancer is breast cancer. In some embodiments, the cancer is gastric cancer. In some embodiments, the cancer is esophageal cancer. In some embodiments, the cancer is ovarian cancer. In some embodiments, the cancer is endometrial cancer. In some embodiments, the cancer is bladder cancer. In some embodiments, the cancer is lung cancer. In some embodiments, the cancer is cervical cancer. In some embodiments, the cancer is colon cancer. In some embodiments, the cancer is head and neck cancer.

In some embodiments, the cancer is HER2+ breast cancer, HER2+ gastric cancer, HER2+ esophageal cancer, HER2+ ovarian cancer, HER2+ endometrial cancer, HER2+ bladder cancer, HER2+ lung cancer, HER2+ cervical cancer, HER2+ colon cancer, HER2+ head and neck cancer. In some embodiments, the cancer is HER2+ breast cancer. In some embodiments, the cancer is HER2+ gastric cancer. In some embodiments, the cancer is HER2+ esophageal cancer. In some embodiments, the cancer is HER2+ ovarian cancer. In some embodiments, the cancer is HER2+ endometrial cancer. In some embodiments, the cancer is HER2+ bladder cancer. In some embodiments, the cancer is HER2+ lung cancer. In some embodiments, the cancer is HER2+cervical cancer. In some embodiments, the cancer is HER2+ colon cancer. In some embodiments, the cancer is HER2+ head and neck cancer.

In some embodiments, the cancer is HER2 low breast cancer, HER2 low gastric cancer, HER2 low esophageal cancer, HER2 low ovarian cancer, HER2 low endometrial cancer, HER2 low bladder cancer, HER2 low lung cancer, HER2 low cervical cancer, HER2 low colon cancer, HER2 low head and neck cancer. In some embodiments, the cancer is HER2 low breast cancer. In some embodiments, the cancer is HER2 low gastric cancer. In some embodiments, the cancer is HER2 low esophageal cancer. In some embodiments, the cancer is HER2 low ovarian cancer. In some embodiments, the cancer is HER2 low endometrial cancer. In some embodiments, the cancer is HER2 low bladder cancer. In some embodiments, the cancer is HER2 low lung cancer. In some embodiments, the cancer is HER2 low cervical cancer. In some embodiments, the cancer is HER2 low colon cancer. In some embodiments, the cancer is HER2 low head and neck cancer.

In some embodiments, the cancer is metastatic. In some embodiments, the cancer is advanced.

### EXAMPLES

### Example 1. Combination of a Deuterated ETBR Antagonist, ICPI, and CAR-T Therapy, in an In Vivo Model

### Generation of Anti-HER2 Mouse CAR-T Cells

Lentivirus for expression of an anti-HER2 CAR was produced by co-transfecting 293T-17 cells with lentiviral vector pLVX-EF1α-CAR-IRES-ZsGreen1 together with the packaging plasmids pSPAX2 and pMD2.G. Splenocytes from 6-week old female BALB/c mice were isolated by density-gradient centrifugation using a lymphocyte separation medium, Histopaque, activated in 24-well plates at a density of 1×10⁶ cell/ml using a mouse T Cell Activation/Expansion Kit for 2-3 days, and then transduced with pLVX-EF1α-CAR-IRES-ZsGreen1 lentivirus at the multiplicity of infection of 20 in the presence of 8 g/mL polybrene by centrifugation at 1,500×g for 2 hours at 32° C. GFP+ transduced T cells were analyzed by flow cytometry to evaluate the lentiviral transduction efficiency.

To assess the cytotoxicity of anti-HER2 mouse CAR-T against HER2 positive breast cancer cells in vitro, CAR-T cells were incubated with 4T1-HER2 cells in triplicate at the effector: target ratios of 2:1, 4:1, and 8:1. Specific lactate dehydrogenase (LDH) released into the culture supernatant from the target cells was determined using the LDH cytotoxicity detection kit according to the manufacturer's instructions. In addition, IL-2 secretion of anti-HER2 mouse CAR-T cells was assayed by incubating CAR-T cells with 4T1-HER2 cells in triplicate at the E:T ratios of 2:1, 4:1, and 8:1 for 24 hours. IL-2 concentrations in culture supernatants will be analyzed by using IL-2 ELISA kit (R&D Systems) according to the manufacturer's instruction.

### GENERATION of 4T-1-HER2 Mammary Tumors in Syngeneic Female BALB/c Mice

4T1-HER2 cells (4×10⁶), were resuspended in 0.1 mL PBS and mixed with 0.1 mL Matrigel and injected subcutaneously into the right back region of BALB/c female mice (6-week-old, weighed 17-20 g). Tumor growth was monitored by caliper measurement every three days.

Treatment of 4T-1-HER2 Mammary Tumors with Anti-HER2 CAR-T and BQ-788-B,

About 14 days after 4T-1-HER2 cell injection, when the tumor diameter reaches about 6 mm in diameter, mice were randomized into eight groups for treatments (6 mice per group).

Blank T or CAR-T cells (1×10⁷ cells/mice) were injected intravenously into each mouse on day 11 and day 18 post-tumor inoculation. BQ-788-B (24 g) or PBS was administered via subcutaneous injection into each mouse, starting on day 10, every two or three days for a total of 9 injections. All mice were injected intraperitoneally with 20,000 IU IL-2 once every 2 days from day 14 to day 34 (Note: day 0 is defined as when 4T1-HER2 cells are injected into mice). Anti-PD1 or isotype control Ig (230 ug/mouse) was injected IP every 4 days, starting on day 11 post tumor inoculation (4 injections total). The eight treatment groups are listed below:
1) Blank T+PBS+isotype
2) CAR-T+PBS+isotype
3) BQ-788-B+Blank T+isotype
4) Blank T+PBS+anti-PD1
5) BQ-788-B+CAR-T+ isotype
6) CAR-T+PBS+anti-PD1
7) BQ-788-B+Blank T+anti-PD1
8) BQ-788-B+CAR-T+ anti-PD1

Tumor growth was monitored by caliper measurement every three days. On the 30^{th} day after 4T1-HER2 cell injection, all mice were euthanized by asphyxiation with CO₂ followed by cervical dislocation. All tumors were dissected out and photographed before fixation in 10% formalin.

### Results

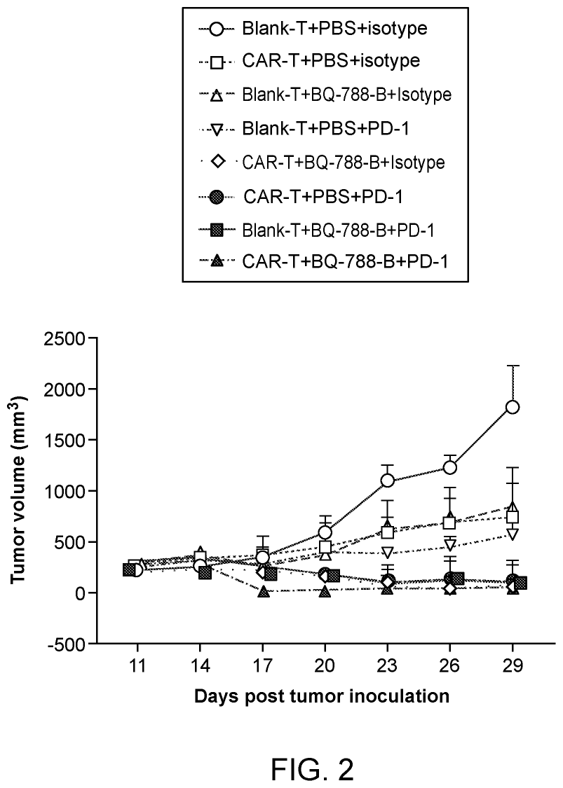

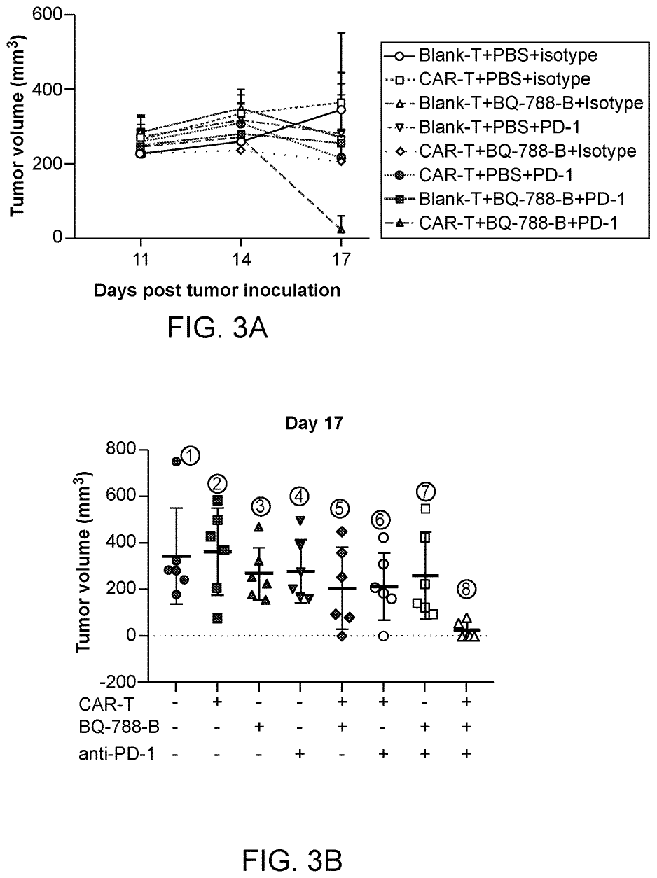

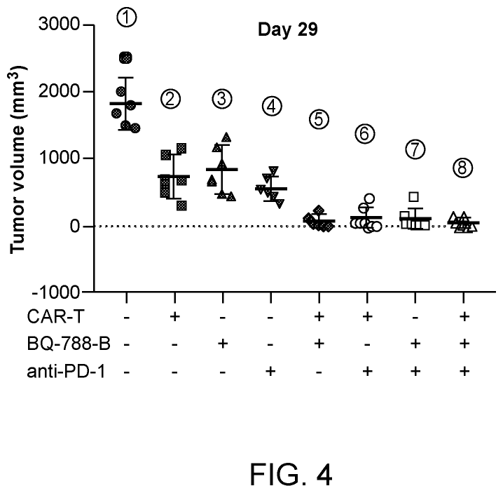

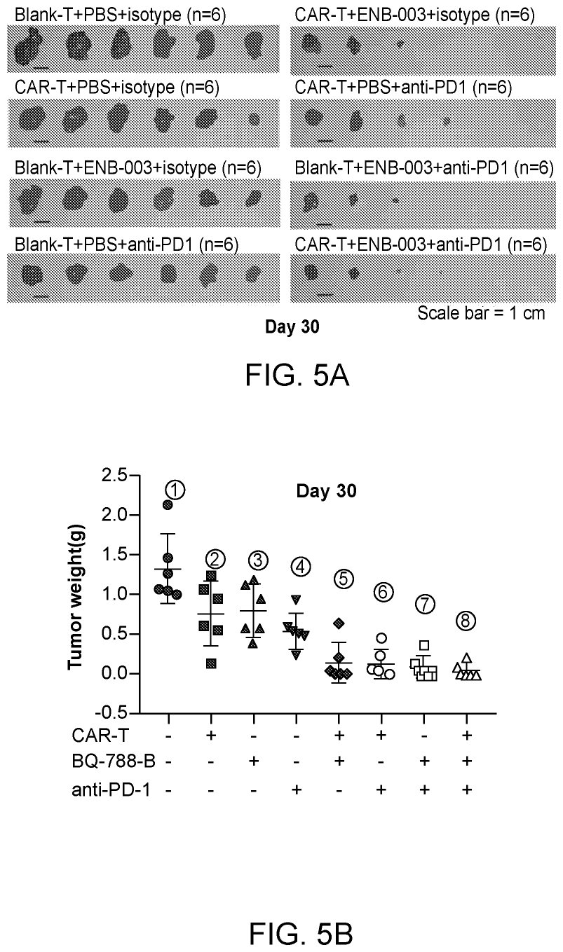

All treatment groups showed a reduction in overall tumor growth compared to Blank-T+PBS+isotype control (FIG. 2). At day 17, the combination of CAR-T, BQ-788-B, and anti-PD-1 therapy demonstrated the greatest reduction in tumor growth, especially compared to control or CAR-T alone (FIGS. 3A and 3B). At later time points, addition of BQ-788-B enhanced tumor suppression in mice treated with either anti-PD1 or CAR-T therapy. At day 29 (FIG. 4), groups treated with BQ-788-B in combination with anti-PD1 and/or CAR-T therapy had significantly reduced tumor volumes compared to control, CAR-T alone, or PD-1 alone. At day 30 (FIGS. 5A and 5B), groups treated with BQ-788-B in combination with anti-PD1 and/or CAR-T therapy had significantly reduced tumor weights compared to control, CAR-T alone, or PD-1 alone. These experiments demonstrate that the addition of BQ-788-B significantly enhances the anti-tumor efficacy of CAR-T cells, of anti-PD-1 antibody therapy and of combinations of CAR-T and anti-PD-1 antibody therapy.

### Example 2. Treatment of a Solid Tumor in a Human Subject with a Combination Therapy Including a CAR-Expressing Immune Cell and a Deuterated ETBR Antagonist

A human patient suffering from a target-expressing cancer, e.g., a HER2-expressing cancer, e.g., breast cancer, is administered a combination therapy regimen including a CAR-expressing immune cell (e.g., CAR-T or CAR-NK cell) and a deuterated ETBR antagonist (e.g., BQ-788-A, BQ-788-B, or BQ-788-C) according to a method disclosed herein. The treatment reduces a tumor volume or mass, or eradicates the tumor in the patient.

### Example 3. Treatment of a Solid Tumor in a Human Subject with a Combination Therapy Including a CAR-Expressing Immune Cell, an Immune Checkpoint Inhibitor, and a Deuterated ETBR Antagonist

A human patient suffering from a cancer, e.g., a HER2-expressing cancer, e.g., breast cancer, is administered a combination therapy regimen including a CAR-expressing immune cell (e.g., CAR-T or CAR-NK cell), an ICPI (e.g., an anti-PD1 antibody), and a deuterated ETBR antagonist (e.g., BQ-788-A, BQ-788-B, or BQ-788-C) according to a method disclosed herein.. The treatment reduces a tumor volume or mass, or eradicates the tumor in the patient.

While some embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treating a target-expressing cancer in a subject in need thereof, the method comprising administering to the subject a combination comprising:
(a) a chimeric antigen receptor (CAR)-expressing immune cell, wherein the CAR specifically binds the target, and
(b) an effective amount of an ETBR antagonist.

2. In a method of treating a target-expressing cancer in a subject in need thereof with a chimeric antigen receptor (CAR)-expressing immune cell, wherein the CAR specifically binds the target, the improvement comprising co-administering to the subject an effective amount of an ETBR antagonist.

3. The method of claim 1 or 2, wherein the target is a tumor-associated antigen.

4. The method of claim 3, wherein the tumor-associated antigen is HER2.

5. The method of any one of claims 1 to 4, further comprising co-administering to the patient an effective amount of an immune checkpoint inhibitor (ICPI).

6. A method of treating a target-expressing cancer in a subject in need thereof, the method comprising administering to the subject a combination comprising:
(a) a chimeric antigen receptor (CAR)-expressing immune cell, wherein the CAR specifically binds the target,
(b) an immune checkpoint inhibitor (ICPI); and
(c) an effective amount of an ETBR antagonist.

7. In a method of treating a target-expressing cancer in a subject in need thereof with a chimeric antigen receptor (CAR)-expressing immune cell, wherein the CAR specifically binds the target, and an immune checkpoint inhibitor (ICPI), the improvement comprising co-administering to the subject an effective amount of an ETBR antagonist.

8. The method of claim 6 or 7, wherein the target is a tumor-associated antigen.

9. The method of claim 8, wherein the tumor-associated antigen is HER2.

10. A method of treating a HER2-expressing cancer in a subject in need thereof, the method comprising administering to the subject a combination comprising:
(a) a chimeric antigen receptor (CAR)-expressing immune cell, wherein the CAR specifically binds HER2, and
(b) an effective amount of a deuterated ETBR antagonist.

11. In a method of treating a HE-2-expressing cancer in a subject in need thereof with a chimeric antigen receptor (CAR)-expressing immune cell, wherein the CAR specifically binds HER2, the improvement comprising co-administering to the subject an effective amount of an ETBR antagonist.

12. The method of claim 10 or 11, further comprising co-administering to the patient an effective amount of an immune checkpoint inhibitor (ICPI).

13. The method of any one of claims 5-9 and 12, wherein the ICPI is an anti-PD1 antibody, an anti-PD-L1 antibody, an anti-CTLA4 antibody, or a variant or functional fragment thereof.

14. The method of claim 13, wherein the ICPI is an anti-PD1 antibody.

15. The method ofany one of claims 1-14, wherein the ETBR antagonist is a deuterated ETBR antagonist.

16. The method of claim 15, wherein the deuterated ETBR antagonist is a compound of formula (1): 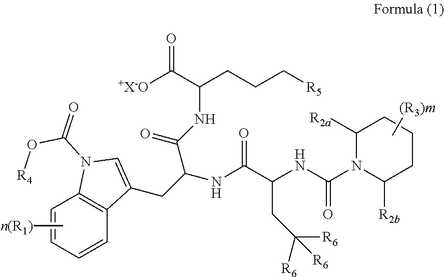
or a stereoisomer thereof, wherein:
n is an integer from 0-4;
in is an integer from 0-3;
X is a positively charged counterion;
R₁ and R₃ are independently —H, -D, —CH₃, —CH₂D, —CHD₂, or —CD₃;
R₂ₐ, R_{2b}, R₄, R₅, and R₆ are independently —CH₃, —CH₂D, —CD₂, or —CD₃; and
at least one of R₁, R₂ₐ, R_{2b}, or R₃ comprises deuterium.

17. The method of claim 16, wherein the compound is a compound of Formula (1a): 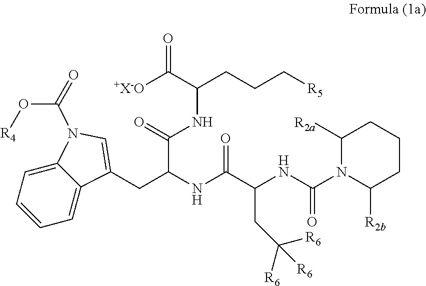
or a stereoisomer thereof.

18. The method of claim 16, wherein the compound is a compound of Formula (2): 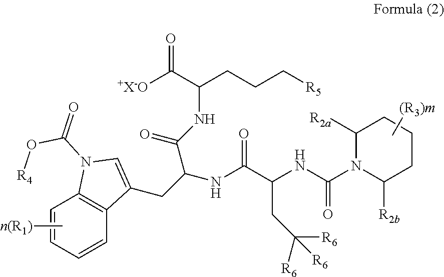
or a stereoisomer thereof.

19. The method of claim 16, wherein the compound is a compound of Formula (2a): 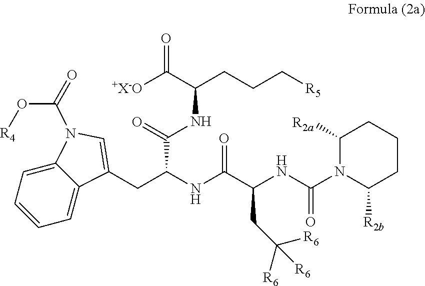
or a stereoisomer thereof.

20. The method of claim 16, wherein the compound is a compound of Formula (3): 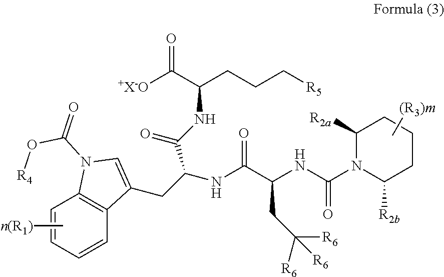
or a stereoisomer thereof.

21. The method of claim 16, wherein the compound is a compound of Formula (4): 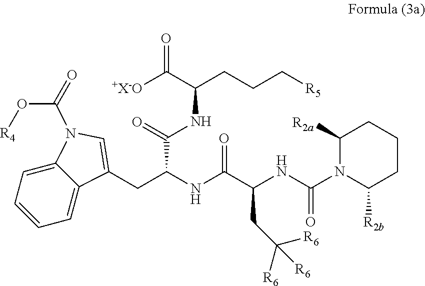
or a stereoisomer thereof.

22. The method of claim 16, wherein the compound is a compound of Formula (4): 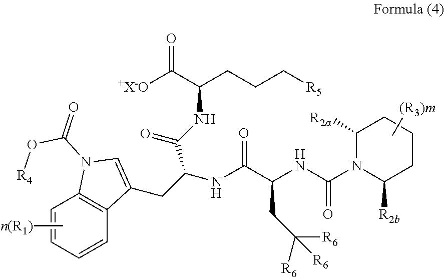
or a stereoisomer thereof.

23. The method of claim 16, wherein the compound is a compound of Formula (4a): 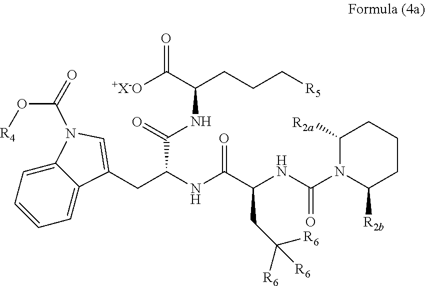
or a stereoisomer thereof.

24. The method of claim 16, wherein the compound is a compound of Formula (5): 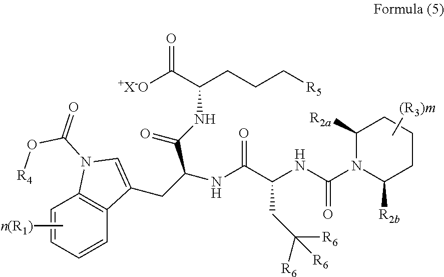
or a stereoisomer thereof.

25. The method of claim 16, wherein the compound is a compound of Formula (5a): 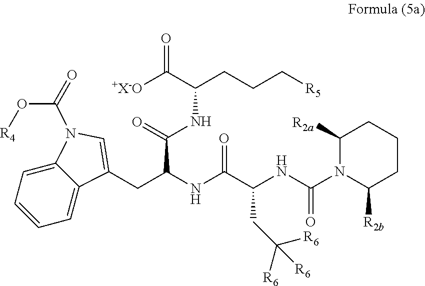
or a stereoisomer thereof.

26. The method of claim 16, wherein the compound is a compound of Formula (6): 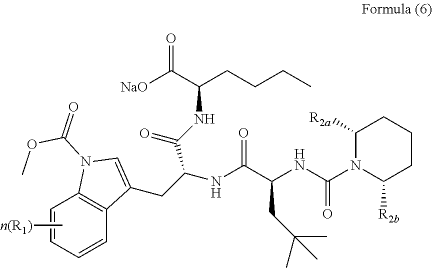
or a stereoisomer thereof.

27. The method of claim 16, wherein the compound is a compound of Formula (6a): 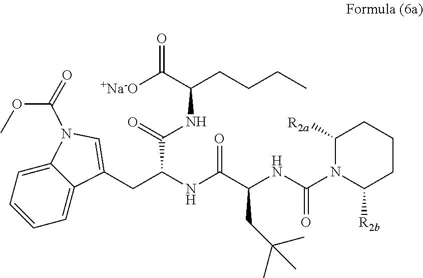
or a stereoisomer thereof.

28. The method of any one of claims 16, 18, 20, 22, 24 and 26, wherein n is 1, R₁ is -D, and R₂ₐ and R_{2b} are each —CH₃.

29. The method of any one of claims 16-27, wherein R₂ₐ is —CH₃, and R_{2b} is —CH₂D.

30. The method of any one of claims 16-27, wherein R₂ₐ and R_{2b} are each —CH₂D.

31. The method of claim 16, wherein n is 1, R₁ is -D; and R₂ₐ and R_{2b} are each —CH₁₂D.

32. The method of any one of claims 1-31, wherein the deuterated ETBR antagonist is selected from: 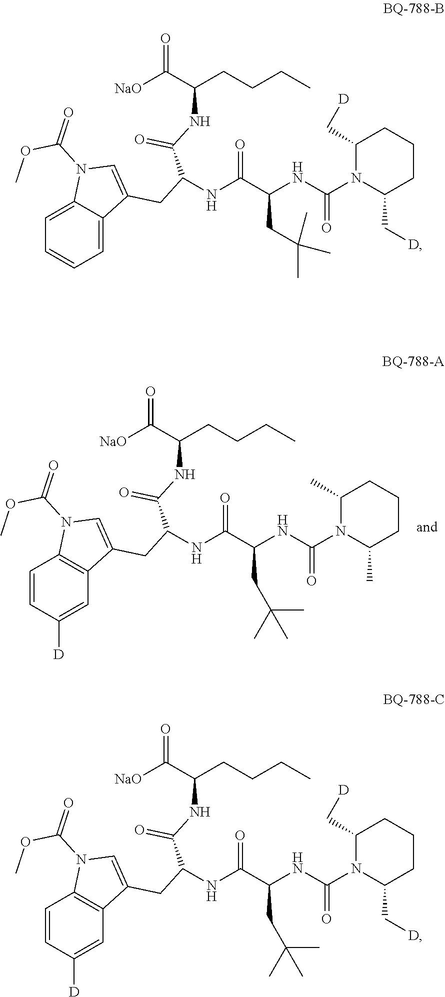
or a stereoisomer thereof.

33. The method of any one of claims 1-32, wherein the ETBR antagonist is: 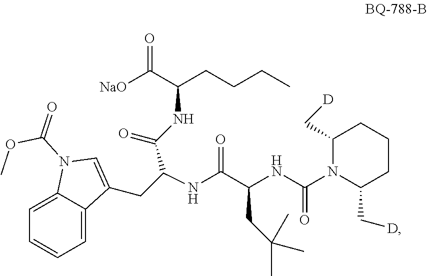
or a stereoisomer thereof.

34. The method of any one of claims 1-33, wherein the ETBR antagonist is in a pharmaceutically acceptable excipient that comprises dimethyl sulfoxide (DMSO).

35. The method of any one of claims 1-34, wherein the CAR-expressing immune cell is a CAR-T cell or a CAR-NK cell.

36. The method of claim 35, wherein the CAR-expressing immune cell is a CAR-T cell.

37. The method of any one of claims 1-36, wherein the cancer is an ETBR-related cancer.

38. The method of any one of claims 1-37, wherein the cancer is selected from the group consisting of: breast cancer, gastric cancer, esophageal cancer, ovarian cancer, endometrial cancer, bladder cancer, lung cancer, cervical cancer, colon cancer, and head and neck cancer.

39. The method of any one of claims 1-9 and 13-32 wherein the cancer is a HER2-expressing cancer.

40. The method of claim 39, wherein the HER2-expressing cancer is a HER2+ cancer.

41. The method of claim 39, wherein the HER2-expressing cancer is a HER2-low cancer.

42. The method of any one of claims 39-41, wherein the HER2-expressing cancer is selected from the group consisting of: breast cancer, gastric cancer, esophageal cancer, ovarian cancer, endometrial cancer, bladder cancer, lung cancer, cervical cancer, colon cancer, and head and neck cancer.
